(19) United States
(12) Patent Application Publication
(10) Pub. No.: US 2022/0175781 A1
(43) Pub. Date: Jun. 9, 2022

(54) CD40L COMPOSITIONS AND METHODS FOR TUNABLE REGULATION

(71) Applicant: Obsidian Therapeutics, Inc., Cambridge, MA (US)

(72) Inventors: Michael SCHEBESTA, Cambrdige, MA (US); Michelle Lois FLEURY, Cambridge, MA (US); Kutlu Goksu ELPEK, Cambridge, MA (US); Elizabeth Jane WEISMAN, Jamaica Plain, MA (US); Vipin SURI, Belmont, MA (US); Dexue SUN, Cambridge, MA (US); Dan Jun LI, Cambridge, MA (US); Steven Mark SHAMAH, Acton, MA (US); Michael Joseph BRISKIN, Lexington, MA (US); Celeste RICHARDSON, Brookline, MA (US); Tariq A. KASSUM, Boston, MA (US); Michelle Lynn OLS, Northborough, MA (US); Brian DOLINSKI, Cambridge, MA (US); Mara Christine INNISS, Beverly, MA (US); Emily BRIDEAU, Cambridge, MA (US); Jennifer Leah GORI, Jamaica Plain, MA (US); Dhruv Kam SETHI, Westwood, MA (US)

(73) Assignee: Obsidian Therapeutics, Inc., Cambridge, MA (US)

(21) Appl. No.: 17/436,997

(22) PCT Filed: Mar. 6, 2020
(86) PCT No.: PCT/US2020/021582

§ 371 Date: Sep. 7, 2021

Related U.S. Application Data
(60) Provisional application No. 62/860,356, filed on Jun. 12, 2019.
(60) Provisional application No. 62/835,554, filed on Apr. 18, 2019.
(60) Provisional application No. 62/815,404, filed on Mar. 8, 2019.

(51) Int. Cl. A61K 31/519; C12N 9/06; A61K 31/55

(52) U.S. Cl. CPC A61K 31/519; C12N 9/003; C12Y 105/01003; C07K 2319/33; A61K 31/55

(57) ABSTRACT

The present disclosure provides regulatable biocircuit systems, effector modules and compositions for cancer immunotherapy. Methods for inducing anti-cancer immune responses in a subject are also provided.

### CROSS REFERENCE TO RELATED APPLICATIONS

The present application claims priority to and the benefit of U.S. Provisional Application No. 62/815,404, filed Mar. 8, 2019; U.S. Provisional Application No. 62/835,554, filed Apr. 18, 2019; and U.S. Provisional Application No. 62/860,356, filed Jun. 12, 2019, the contents of each of which are herein incorporated by reference in their entirety.

### REFERENCE TO THE SEQUENCE LISTING

The present application is being filed along with a Sequence Listing in electronic format. The Sequence Listing is provided as a file entitled 2095_1216PCT_ST25.txt, created on Mar. 4, 2020, which is 10.8 MB in size. The information in the electronic format of the sequence listing is incorporated herein by reference in its entirety.

### BACKGROUND

Gene and cell therapies are revolutionizing medicine and offering new promise for the treatment of previously intractable conditions. However, most current technologies do not allow titration of the timing or levels of target protein induction. This has rendered many potential gene and cell therapy applications difficult or impossible to safely and effectively deploy.

Inadequate exogenous and/or endogenous gene control is a critical issue in many gene and cell therapy settings. This lack of tunability also makes it difficult to safely express proteins with narrow or uncertain therapeutic windows or those requiring more titrated or transient expression.

One approach to regulated protein expression or function is the use of drug responsive domains (DRDs), also known as destabilizing domains (DDs). Drug responsive domains are small protein domains that can be appended to a target protein of interest. DRDs render the attached protein of interest unstable in the absence of a DRD-binding ligand and the protein of interest is rapidly degraded by the ubiquitin-proteasome system of the cell. However, when a specific small molecule DRD-binding ligand binds to the DRD, the attached protein of interest is stabilized, and protein function is achieved.

The role of the immune system in tumor control, in particular T cell-mediated cytotoxicity, is well recognized. There is mounting evidence that T cells can control tumor growth and survival in cancer patients, both in early and late stages of the disease. However, tumor-specific T-cell responses are difficult to mount and sustain in cancer patients.

T cell pathways receiving significant attention to date include signaling through cytotoxic T lymphocyte antigen-4 (CTLA-4, CD152) and programmed death ligand 1 (PD-L1, also known as B7-H1 or CD274). Recently however, other molecules that signal through T cell pathways, including CD40 ligand (CD40L), have generated interest as mediators for tumor control.

CD40L (also known as CD154, CD40 ligand, gp39 or TBAM) is a 33 kDa, Type II membrane glycoprotein (Swiss-ProtAcc-No P29965). Additionally, shorter 18 kDa CD40L soluble forms exist, (also known as sCD40L or soluble CD40L). These soluble forms of CD40L are generated by proteolytic processing of the membrane bound protein, but the cellular activity of the soluble species is weak in the absence of higher order oligomerization (e.g., trimerization). CD40L binds and activates CD40.

CD40L is a member of the TNF family of molecules which is primarily expressed on activated T cells (including Th0, Th1, and Th2 subtypes), and forms homotrimers similar to other members of this family. Further, CD40L has also been found expressed on mast cells, and activated basophils and eosinophils. CD40L binds to its receptor CD40 on antigen-presenting cells (APC), which leads to many effects depending on the target cell type. In general, CD40L plays the role of a costimulatory molecule and induces activation in APC in association with T cell receptor stimulation by MHC molecules on the APC. CD40L also may bind to B cells, monocytes, macrophages, platelets, neutrophils, dendritic cells, endothelial cells, and αSMC (smooth muscle cells). Binding of CD40L to CD40 expressed on dendritic cells may promote dendritic cell (DC) licensing. DCs may be converted to a functional state by an antigen-specific T helper cell in order to activate cytotoxic CD8+ T cells, a process referred to as DC licensing. CD40 engagement on DCs results in DC stimulation as evidenced by the surface expression of costimulatory and MHC molecules; proinflammatory cytokine production (e.g. IL12 and TNF) as well as epitope spreading, all immune responses that are believed to assist in tumor eradication and anti-tumor effects.

Despite the significant progress made over the past decade in developing strategies for combatting cancer and other diseases, patients with advanced, refractory and metastatic disease have limited clinical options. Chemotherapy, irradiation, and high dose chemotherapy have become dose limiting. There remains a substantial unmet need for new less-toxic methods and therapeutics that have better therapeutic efficacy, longer clinical benefit, and improved safety profiles, particularly for those patients with advanced disease or cancers that are resistant to existing therapeutics.

### SUMMARY

The present disclosure provides novel protein domains displaying small molecule dependent stability. Such protein domains are called drug responsive domains (DRDs). In the absence of its binding ligand, the DRD is destabilizing and causes degradation of a payload or protein of interest (POI) (used interchangeably herein) fused to the DRD, while in the presence of its binding ligand, the operably linked DRD and payload can be stabilized, and its stability is dose dependent.

Provided herein are compositions which include an effector module. The effector module may include a stimulus response element (SRE) which is operably linked to one or more payloads. In various embodiments, the SRE comprises a drug responsive domain (DRD), or consists essentially of a drug responsive domain (DRD), or consists of a drug responsive domain (DRD). As used herein, an SRE may also be referred to as a DRD, or DD.

Compositions that are exemplified herein, include, but are not limited to a composition comprising an effector module, said effector module comprising a stimulus response element (SRE) operably linked to a first payload, wherein said first payload comprises human CD40L (SEQ ID NO: 3820) or a mutant CD40L comprising one or more mutations selected from Y170G, Y172G, H224G, G226F, G226H, G226W, or G227F relative to SEQ ID NO: 3820, said payload is attached, appended or associated with said SRE. The compositions illustrated above may comprise a DRD, wherein the DRD comprises, in whole or in part, a ER, an ecDHFR, a FKBP, a PDE5, or an hDHFR protein, wherein the DRD further comprises one or more mutations in said amino acid sequence of the ER, ecDHFR, FKBP, PDE5, or hDHFR protein.

Exemplary CD40L payloads described herein may include one or more mutations relative to SEQ ID NO: 3820, such as but not limited to Y170G, Y172G, H224G, G226F, G226H, G226W, or G227F. The first payload may include, in whole or in part, the human CD40L (SEQ ID NO: 3820). In some embodiments, the first payload may be the whole CD40L (SEQ ID NO: 3820). Non-limiting examples of payloads comprising the whole CD40L may be CD40L (H224G, G226F) (SEQ ID NO: 6598); CD40L (H224G, G226H) (SEQ ID NO: 6600); CD40L (Y172G, G226F) (SEQ ID NO: 6602); CD40L (Y170G, H224G, G226W) (SEQ ID NO: 6604); or CD40L (H125G, G227F) (SEQ ID NO: 6606).

The SRE described herein may be responsive to or interact with at least one stimulus. In one embodiment, the stimulus may be a small molecule.

The present disclosure provides compositions that include effector modules with SREs derived from the whole or portion of a parent protein, such as ecDHFR and a first payload which includes in whole or in part the human CD40L (SEQ ID NO: 3820). In one embodiment, the SRE includes amino acids 2-159 of ecDHFR. In some embodiments, the SRE may include one or more mutations compared to the parent protein. The SRE may include but is not limited to SEQ ID NO: 6554, 6556, 6558, 6560, 6562, 6564, 6566, 6568, 6570, 6572, 6574, 6576, 6578, 6580, 6582, 6584, 6586, 6588, or 6590.

The SRE described herein may be responsive to or interact with at least one stimulus. In one aspect, the stimulus may be a small molecule such as but not limited to TMP.

Also provided herein are polynucleotides encoding the compositions described herein as well as vectors encoding the polynucleotides, and host cells containing the vectors described herein. The present disclosure also provides pharmaceutical compositions that include the compositions described herein and a pharmaceutically acceptable excipient as well as modified cells or engineered cells expressing the compositions described herein.

Also exemplified herein are methods for the treatment of disease, for example, a subject having cancer in need of such treatment. An exemplary method for treating cancer in accordance with the embodiments of the present disclosure comprises a method of reducing a tumor burden in a subject comprising: (a) administering to the subject a therapeutically effective amount of immune cells as disclosed herein, wherein the immune cells comprise an effector module comprising at least one stimulus response element (SRE), the SRE operably linked to a first payload, wherein said first payload comprises in whole or in part the human CD40L (SEQ ID NO. 3820), or a mutant CD40L thereof; and (b) administering to the subject, a therapeutically effective amount of a stimulus, to modulate the expression of the first payload, thereby reducing the tumor burden. In related embodiments, the effector module may comprise a second payload that is expressed in the immune cells with or without linkage to the same or different DRD as the first payload. In some related aspects, the second payload is a chimeric antigen receptor (CAR), for example, a CD19 CAR as described herein.

In an illustrative example, the present disclosure provides a method of activating dendritic cells in a subject comprising the steps of (a) administering to the subject one or more immune cells, said one or more immune cells comprising an effector module, the effector module having at least one stimulus response element (SRE) operably linked to a first payload, wherein said first payload comprises in whole or in part the human CD40L (SEQ ID NO. 3820), or a mutant thereof; wherein the immune cell is a T cell; (b) administering to the subject, a therapeutically effective amount of a stimulus wherein the stimulus is a ligand, to modulate the expression of the first payload; and (c) measuring dendritic cell activation marker, IL12 in the subject in response to the ligand to measure dendritic cell activation.

### DETAILED DESCRIPTION

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### A. Compositions

The present disclosure provides modified cells, nucleic acid molecules, vectors, and cell and gene therapies in which the timing or levels of a native therapeutic protein can be regulated through administration of an oral small molecule drug. Additionally, the present disclosure provides compositions, systems and methods for tunable expression of a protein of interest (POI, also referred to herein as “payload” which may be used interchangeably). The compositions relate to systems for the tunable expression of a protein of interest in a cell and agents that induce the translation of a polynucleotide encoding at least one drug responsive domain (DRD) operably linked to at least one protein of interest. Compositions provided by the present disclosure include nucleic acid molecules, polypeptides, and modified cells related to systems for the tunable expression of a protein of interest for use in treating a disease in the presence of a ligand that stabilizes the DRD. Methods related to tunable systems for protein expression that are provided by the present disclosure include methods of producing modified cells and methods of treating or preventing disease.

In various embodiments, the present disclosure provides a method for treating a disease in a subject in need thereof, the method comprising: a. providing a population of cells; b. introducing at least one nucleic acid into at least one cell in the population of cells, wherein the nucleic acid molecule comprises at least one nucleic acid sequence that encodes a protein of interest operably linked to a drug responsive domain (DRD); c. Delivering the cell into the subject; and d. administering a ligand to the subject that stabilizes the DRD sufficiently to enable expression of the protein of interest in the at least one cell; wherein expression of the protein of interest is regulated by the presence of ligand in the subject, and the amount and/or duration of ligand administration is sufficient to produce a therapeutically effective amount of the protein of interest in the at least one cell in the population of cells.

1. Tunable Protein Expression Systems

In general, a stimulus response element (SRE) comprising the DRD may be operably linked to a payload construct which could be any payload (e.g., an immunotherapeutic agent), to form an effector module. The SRE, when activated by a particular stimulus, stabilizing ligand or simply ligand (used interchangeably herein), e.g., a small molecule, can produce a signal or outcome, to regulate transcription, translation, and/or protein levels of the linked payload in the engineered cell. The tunable expression of the payload can be modulated either up or down by providing or excluding a stabilizing ligand which stabilizes the DRD to effect tunable expression of the operably linked payload. In various embodiments, the present disclosure provides polynucleotides that encode a SRE that tune expression levels and activities of any agents used for immunotherapy.

As used herein, a “biocircuit” or “biocircuit system” is defined as a circuit within or useful in biologic systems comprising a stimulus and at least one effector module responsive to a stimulus, where the response to the stimulus produces at least one signal or outcome within, between, as an indicator of, or on a biologic system. Biologic systems are generally understood to be any cell, tissue, organ, organ system or organism, whether animal, plant, fungi, bacterial, or viral. It is also understood that biocircuits may be artificial circuits which employ the stimuli or effector modules taught by the present disclosure and effect signals or outcomes in acellular environments such as with diagnostic, reporter systems, devices, assays or kits. The artificial circuits may be associated with one or more electronic, magnetic, or radioactive components or parts.

2. Effector Modules and Stimulus Response Elements (SREs)

The systems, compositions and methods of the present disclosure include at least one stimulus response element as a component of an effector module system. As used herein, an “effector module” is a single or multi-component construct or complex comprising at least (a) one or more stimulus response elements and (b) one or more payloads (e.g. payloads of interest or proteins of interest (POIs)). In one embodiment, the DRD of the SRE comprises, in whole or in part, a ER, an ecDHFR, a FKBP, a PDE5, or an hdhdr hDHFR protein, wherein the DRD further comprises one or more mutations in said amino acid sequence of the ER, ecDHFR, FKBP, PDE5, or hDHFR protein. In one embodiment, the payload comprises human CD40L (SEQ ID NO: 3820) or a mutant CD40L comprising one or more mutations selected from Y170G, Y172G, H224G, G226F, G226H, G226W, or G227F relative to SEQ ID NO: 3820.

As used herein a “stimulus response element (SRE)” is a component of an effector module which is joined, attached, operably linked to, or associated with one or more payloads of the effector module and in some instances, is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1% and 100% or a factored increase or decrease such as 2-fold, 3-fold, 4-fold, 5-fold, 10-fold or more.

In some embodiments, the present disclosure provides methods for modulating protein expression, function or level. In some aspects, the modulation of protein expression, function or level refers to modulation of expression, function or level by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

3. Characterization of Ligand-Dependent Activity of a Tunable Protein Expression System

Ligand-dependent activity of a tunable protein expression system may be characterized by various methods.

In some embodiments, ligand-dependent activity of a tunable protein expression system is characterized by ligand-dependent regulation of a DRD. In some embodiments, ligand-dependent activity of a tunable protein expression system is characterized by ligand dose-dependent regulation of a DRD. Ligand-dependent regulation of a DRD transcription factor polypeptide may be characterized by various methods. In some aspects, ligand-dependent regulation of a DRD may be assessed by measuring the levels of the DRD, the operably linked protein of interest, or both, such as by an immunoassay targeted to measure the levels of the DRD, protein of interest, or both.

In some embodiments, ligand-dependent activity of a tunable protein expression system is characterized by ligand-dependent expression of the payload operably linked to a DRD. Expression of the payload may be assessed by various methods. In some aspects, expression of the payload is assessed by measuring protein/polypeptide levels.

In some embodiments, a tunable protein expression system may be compared to a control tunable protein expression system that lacks a DRD. In some embodiments, ligand-dependent activity of a tunable protein expression system may be analyzed or characterized relative to the activity of a tunable protein expression system comprising a payload containing construct that lacks a DRD.

In various embodiments, the tunable protein expression system provides for the tunable expression of a protein of interest or payload (used interchangeably herein). In various embodiments, a nucleic acid sequence of the present disclosure comprises a nucleotide sequence which encodes the protein of interest and is operably linked to a nucleotide sequence which encodes a DRD. The nucleotide sequence which encodes the DRD may be positioned upstream or downstream of the nucleic acid sequence which encodes the payload.

When a cell or organism comprising a DRD is exposed to an exogenous stabilizing ligand, the DRD is stabilized. The stabilized DRD and any polypeptide sequences positioned upstream or downstream from the DRD is also stabilized and not transported to the ubiquitin proteasome degradation pathway. In the absence of the exogenous stabilizing ligand, the DRD and any operably linked polypeptide sequences operably linked to the DRD upstream and/or downstream from the DRD is degraded and eliminated in the cell. Thus, both the amount and the timing of protein expression can be controlled by administering the exogenous stabilizing ligand to the cell or organism.

In various embodiments, the DRD and the protein of interest are typically operably linked or may be separated by one or more intervening nucleotide, peptide, polypeptide or protein sequences, for example, a linker, a signal sequence, a leader sequence, a transmembrane domain, an intra tail domain of a cleavage site. In various embodiments, a first polynucleotide may include a first nucleic acid sequence that encodes a drug responsive domain (DRD) and a second nucleic acid sequence that encodes a protein of interest. In such embodiments, the protein of interest as described herein in the cell, is operably linked to the DRD. In addition, the cell may also include other optional nucleotide, peptide, polypeptide or protein sequences which may be operably linked to the DRD, the protein of interest, or both.

In some embodiments, a vector comprises the polynucleotides described herein. In some embodiments, the vector comprises at least a first polynucleotide may include a first nucleic acid sequence that encodes a payload; a second nucleic acid sequence that encodes a drug responsive domain (DRD) which is operably linked to the payload. Optionally, in some embodiments, the first polynucleotide and/or vector may comprise additional components of the tunable protein expression system, for example, for monocistronic and/or bicistronic expression of the one or more payloads and one or more DRDs, for example, IRES sequences and cleavage sites, with optional intervening peptide or polypeptide sequences positioned upstream or downstream from the payload; or upstream, or downstream from the DRD.

In some embodiments, the vectors also possess an origin of replication (ori) which permits amplification of the vector, for example in bacteria. Additionally, or alternatively, the vector includes selectable markers such as antibiotic resistance genes, genes for colored markers and suicide genes, and other regulatory sequences which permit cloning and/or expression in bacteria, viruses and in eukaryotic cells.

4. Drug Responsive Domains (DRDs)

Drug responsive domains (DRDs or DDs) are protein domains that are unstable and are degraded in the absence of ligand, but whose stability is rescued by binding to a corresponding DRD-binding ligand, also referred to herein as a stimulus, or stimulus ligand or simply a ligand. The term drug responsive domain (DRD) is interchangeable with the term destabilizing domain (DD). Drug responsive domains (DRDs) can be appended to a polypeptide or protein of interest and can render the attached polypeptide or protein unstable in the absence of a DRD-binding ligand. DRDs convey their destabilizing property to the attached polypeptide or protein via protein degradation. Without wishing to be bound by any particular theory, it is believed that in the absence of a DRD-binding ligand, the appended or operably linked polypeptide or protein is rapidly degraded by the ubiquitin-proteasome system of a cell. A ligand that binds to or interacts with a DRD can, upon such binding or interaction, modulate the stability of the DRD and any appended or operably linked polypeptide or protein. When a ligand binds its intended DRD, the instability is reversed and function of the appended polypeptide or protein can be restored. The conditional nature of DRD stability allows a rapid and non-perturbing switch from stable protein to unstable substrate for degradation. Moreover, its dependency on the concentration of its ligand further provides tunable control of degradation rates.

In some embodiments, DRDs of the present disclosure may be derived from known polypeptides that are capable of post-translational regulation of proteins. In some embodiments, DRDs of the present disclosure may be developed or derived from known proteins. Regions or portions or domains of wild type proteins may be utilized as DRDs in whole or in part. They may be combined or rearranged to create new peptides, proteins, regions or domains of which any may be used as DRDs or the starting point for the design of further DRDs.

In some embodiments, a DRD may be derived from a parent protein or from a mutant protein having one, two, three, or more amino acid mutations compared to the parent protein. In some embodiments, the parent protein may be selected from, but is not limited to, FKBP; human protein FKBP; human DHFR (hDHFR); E. coli DHFR (ecDHFR); PDE5 (phosphodiesterase 5); and ER (estrogen receptor). Examples of proteins that may be used to develop DRDs and their ligands are listed in Table 1.

TABLE 1
DRDs and their binding ligands

| | | | Nucleic | |
|---|---|---|---|---|
| | | Protein | Acid | |
| | | SEQ ID | SEQ ID | |
| DRD Identifier | Protein | NO: | NO: | Ligands |
| PDE5DD-187 | Human Phosphodiesterase 5 | 1 | 2 | Sildenafil; |
| | (hPDE5) (Uniprot ID: O76074) | | | Vardenafil; |
| | | | | Tadalafil |
| PDE5DD-229 | Human Phosphodiesterase 5 | 23 | 24 | |
| | (hPDE5) Isoform 2 | | | |
| PDE5DD-232 | Human Phosphodiesterase 5 | 25 | 26-27 | |
| | (hPDE5) Isoform 3 | | | |
| PDE5DD-234 | Human Phosphodiesterase 5 | 28 | 29 | |
| | (hPDE5) Isoform X1 | | | |
| hDHFRDD-84 | Human Dihydrofolate reductase | 30 | 31 | Methotrexate |
| | (hDHFR) Isoform 1 (Uniprot ID: | | | (MTX), |
| | P00374.2) | | | Trimethoprim |
| | | | | (TMP) |
| hDHFRDD-87 | Human Dihydrofolate reductase | 32 | | Methotrexate |
| | (hDHFR) Variant | | | (MTX), |
| | | | | Trimethoprim |
| | | | | (TMP) |
| hDHFRDD-88 | Dihydrofolate reductase 2 | 33 | 34 | Methotrexate |
| | (hDHFR2) (DHFRL1) | | | (MTX), |
| | | | | Trimethoprim |
| | | | | (TMP) |
| ecDHFRDD-6 | E. coli Dihydrofolate reductase | 35 | 36 | Methotrexate |
| | (ecDHFR) (Uniprot ID: P0ABQ4) | | | (MTX), |
| | | | | Trimethoprim |
| | | | | (TMP) |
| FKBPDD-8 | FK506 binding protein (FKBP) | 37 | | Shield-1 |
| | (Uniprot ID: P62942) | | | |
| ERDD-4 | Estrogen Receptor (ER) (Uniprot | 42 | | Bazedoxifene, |
| | ID: P03372.2) | | | Raloxifene 4- |
| | | | | hydroxytamoxifen |
| | | | | (4-OHT), |
| | | | | fulvestrant, |
| | | | | oremifene, |
| | | | | lasofoxifene, |
| | | | | clomifene, |
| | | | | femarelle, |
| | | | | ormeloxifene |

In some embodiments, the sequence of a protein used to develop DRDs may comprise all, part of, or a region thereof of a protein sequence in Table 1. In some embodiments, proteins that may be used to develop DRDs include isoforms of proteins listed in Table 1.

In some embodiments, a DRD of the present disclosure is derived from hPDE5. In some embodiments, a DRD of the present disclosure is derived from hPDE5 isoform 2. In some embodiments, a DRD of the present disclosure is derived from hPDE5 isoform 3. In some embodiments, a DRD of the present disclosure is derived from hPDE5 isoform Xl.

In some embodiments, a DRD of the present disclosure is derived from a human dihydrofolate reductase (hDHFR) protein such as, but not limited to, human dihydrofolate reductase 1 (hDHFR1), human dihydrofolate reductase 2 (hDHFR2), or a fragment or variant thereof.

In some embodiments, the DRD may be derived from an hDHFR protein and include at least one mutation. In some embodiments, the DRD may be derived from an hDHFR protein and include more than one mutation. In some embodiments, the DRD may be derived from an hDHFR protein and include two, three, four or five mutations.

In some embodiments, a DRD of the present disclosure is derived from E. coli dihydrofolate reductase (ecDHFR). In some embodiments, the DRD may be derived from an ecDHFR protein and include at least one mutation. In some embodiments, the DRD may be derived from an ecDHFR protein and include more than one mutation. In some embodiments, the DRD may be derived from an ecDHFR protein and include two, three, four or five mutations.

In some embodiments, a DRD of the present disclosure is derived from a FK506 binding protein (FKBP) protein or a fragment or variant thereof. In some embodiments, the DRD may be derived from a FKBP protein and include at least one mutation. In some embodiments, the DRD may be derived from a FKBP protein and include more than one mutation. In some embodiments, the DRD may be derived from an FKBP protein and include two, three, four or five mutations.

In some embodiments, a DRD of the present disclosure is derived from an Estrogen Receptor (ER) protein or a fragment or variant thereof. In some embodiments, the DRD may be derived from an ER protein and include at least one mutation. In some embodiments, the DRD may be derived from an ER protein and include more than one mutation. In some embodiments, the DRD may be derived from an ER protein and include two, three, four or five mutations.

The amino acid sequences of the DRDs encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent protein from which it is derived.

Examples of DRDs of the present disclosure include those derived from: human DHFR, ecDHFR, human estrogen receptor (ER), FKBP, human protein FKBP, and human PDE5. Suitable DRDs, which may be referred to as drug responsive domains or ligand binding domains, are also known in the art. See, e.g., WO2018/161000; WO2018/231759; WO2019/241315; U.S. Pat. Nos. 8,173,792; 8,530,636; WO2018/237323; WO2017/181119; US2017/0114346; US2019/0300864; WO2017/156238; Miyazaki et al., J Am Chem Soc, 134:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLoS Genet. 5:e1000376; and Feng et al. (2015) Elife 4:e10606.

In some embodiments, a DRD of the present disclosure is derived from a human dihydrofolate reductase (hDHFR) protein such as, but not limited to, human dihydrofolate reductase 1 (hDHFR1), human dihydrofolate reductase 2 (hDHFR2), or a fragment or variant thereof.

In some embodiments, the DRD may be derived from an hDHFR protein and include at least one mutation. In some embodiments, the DRD may be derived from an hDHFR protein and include more than one mutation. In some embodiments, the DRD may be derived from an hDHFR protein and include two, three, four or five mutations.

In some embodiments, a DRD of the present disclosure is derived from E. coli dihydrofolate reductase (ecDHFR). In some embodiments, the DRD may be derived from an ecDHFR protein and include at least one mutation. In some embodiments, the DRD may be derived from an ecDHFR protein and include more than one mutation. In some embodiments, the DRD may be derived from an ecDHFR protein and include two, three, four or five mutations.

In some embodiments, a DRD of the present disclosure is derived from a FK506 binding protein (FKBP) protein or a fragment or variant thereof. In some embodiments, the DRD may be derived from a FKBP protein and include at least one mutation. In some embodiments, the DRD may be derived from a FKBP protein and include more than one mutation. In some embodiments, the DRD may be derived from an FKBP protein and include two, three, four or five mutations.

In some embodiments, a DRD of the present disclosure is derived from an Estrogen Receptor (ER) protein or a fragment or variant thereof. In some embodiments, the DRD may be derived from an ER protein and include at least one mutation. In some embodiments, the DRD may be derived from an ER protein and include more than one mutation. In some embodiments, the DRD may be derived from an ER protein and include two, three, four or five mutations.

The amino acid sequences of the DRDs encompassed in the present disclosure have at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence of a parent protein from which it is derived.

Examples of DRDs of the present disclosure include those derived from: human DHFR, ecDHFR, human estrogen receptor (ER), FKBP, human protein FKBP, and human PDE5. Suitable DRDs, which may be referred to as drug responsive domains or ligand binding domains, are also known in the art. See, e.g., WO2018/161000; WO2018/231759; WO2019/241315; U.S. Pat. Nos. 8,173,792; 8,530,636; WO2018/237323; WO2017/181119; US2017/0114346; US2019/0300864; WO2017/156238; Miyazaki et al., J Am Chem Soc, 134:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLoS Genet. 5:e1000376; and Feng et al. (2015) Elife 4:e10606.

In one embodiment, the SRE is derived from a region of parent protein or from a mutant protein. The region of the parent protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the SRE is derived from a parent protein or from a mutant protein and includes a region of the parent protein. The SRE may include a region of the parent protein which is 1%, 5%, 10%, 15%, 20%, 25%, 30%, 35%, 40%, 45%, 50%, 55%, 60%, 65%, 70%, 75%, 80%, 85%, 90%, 95%, 99%, or 100%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, 10-20%, 20-30%, 30-40%, 40-50%, 50-60%, 60-70%, 70-80%, 80-90%, 90-100%, 10-30%, 20-40%, 30-50%, 40-60%, 50-70%, 60-80%, 70-90%, 80-100%, 10-40%, 20-50%, 30-60%, 40-70%, 50-80%, 60-90%, 70-100%, 10-50%, 20-60%, 30-70%, 40-80%, 50-90%, 60-100%, 10-60%, 20-70%, 30-80%, 40-90%, 50-100%, 10-70%, 20-80%, 30-90%, 40-100%, 10-80%, 20-90%, 30-100%, 10-90%, 20-100%, 25-50%, 50-75%, or 75-100% of the parent protein or mutant protein.

In one embodiment, the SRE is derived from a parent protein or from a mutant protein and may have 1%, 5%, 10%, 15%, 20%, 25%, 30%, 35%, 40%, 45%, 50%, 55%, 60%, 65%, 70%, 75%, 80%, 85%, 90%, 95%, 99%, or 100%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, 95-100%, 10-20%, 20-30%, 30-40%, 40-50%, 50-60%, 60-70%, 70-80%, 80-90%, 90-100%, 10-30%, 20-40%, 30-50%, 40-60%, 50-70%, 60-80%, 70-90%, 80-100%, 10-40%, 20-50%, 30-60%, 40-70%, 50-80%, 60-90%, 70-100%, 10-50%, 20-60%, 30-70%, 40-80%, 50-90%, 60-100%, 10-60%, 20-70%, 30-80%, 40-90%, 50-100%, 10-70%, 20-80%, 30-90%, 40-100%, 10-80%, 20-90%, 30-100%, 10-90%, 20-100%, 25-50%, 50-75%, or 75-100% identity to the parent protein or mutant protein.

In one embodiment, the effector modules and/or SREs of the present disclosure may include at least one drug responsive domain (DRD). The effector modules and/or SRE may include 1, 2, 3, 4, 5, 6, 7, 8, 9, 10 or more than 10 DRDs. When there are more than one DRDs, each of the DRDs may be derived from the same parent protein (e.g., PDE5), from different parent proteins (e.g., PDE5 and hDHFR), may be a fusion of two different parent proteins, or may be artificial.

In one embodiment, the effector modules and/or SREs of the present disclosure may include 2 DRDs. In one embodiment, the effector modules and/or SREs of the present disclosure may include 3 DRDs. In one embodiment, the effector modules and/or SREs of the present disclosure may include 4 DRDs. In one embodiment, the effector modules and/or SREs of the present disclosure may include 5 DRDs. In one embodiment, the effector modules and/or SREs of the present disclosure may include 6 DRDs. In one embodiment, the effector modules and/or SREs of the present disclosure may include 7 DRDs. In one embodiment, the effector modules and/or SREs of the present disclosure may include 8 DRDs. In one embodiment, the effector modules and/or SREs of the present disclosure may include 9 DRDs. In one embodiment, the effector modules and/or SREs of the present disclosure may include 10 DRDs. The DRDs may be derived from any parent protein known in the art and/or described herein. In some embodiments the DRDs are derived from the same parent protein (e.g., PDE5). In some embodiments the DRDs are derived from different regions of the same parent protein (e.g., amino acid 535-860 and amino acid 590-836 of PDE5). In some embodiments, the DRDs are derived from different parent proteins (e.g., PDE5 and hDHFR).

5. Human Dihydrofolate Reductase (hDHFR) Derived Drug Responsive Domains (DRDs)

In one embodiment, the SRE may include at least one drug responsive domain (DD) derived from a human dihydrofolate reductase (hDHFR) protein such as, but not limited to, human dihydrofolate reductase 1 (hDHFR1), human dihydrofolate reductase 2 (hDHFR2), or a fragment or variant thereof. As a non-limiting example, the SRE comprises at least one DRD derived from hDHFR1. As a non-limiting example, the SRE comprises at least one DRD derived from hDHFR2.

In some embodiments, DRDs of the disclosure may be derived from human dihydrofolate reductase (hDHFR). hDHFR is a small (18 kDa) enzyme that catalyzes the reduction of dihydrofolate and plays a vital role in variety of anabolic pathway. Dihydrofolate reductase (DHFR) is an essential enzyme that converts 7,8-dihydrofolate (DHF) to 5,6,7,8, tetrahydrofolate (THF) in the presence of nicotinamide adenine dihydrogen phosphate (NADPH). Anti-folate drugs such as methotrexate (MTX), a structural analogue of folic acid, which bind to DHFR more strongly than the natural substrate DHF, interferes with folate metabolism, mainly by inhibition of dihydrofolate reductase, resulting in the suppression of purine and pyrimidine precursor synthesis. Other inhibitors of hDHFR such as folate, TQD, Trimethoprim (TMP), epigallocatechin gallate (EGCG) and ECG (epicatechin gallate) can also bind to hDHFR and regulates its stability.

In one embodiment, the SRE comprises a region of the hDHFR protein. The region of the hDHFR protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the DRD may be derived from an hDHFR protein and include at least one mutation. Non-limiting examples of mutations include M1del, V2A, C7R, I8V, V9A, A10T, A10V, Q13R, N14S, G16S, I17N, I17V, K19E, N20D, G21E, G21T, D22S, L23S, P24S, L28P, N30D, N30H, N30S, E31D, E31G, F32M, R33G, R33S, F35L, Q36F, Q36K, Q36R, Q36S, R37G, M38T, M38V, T40A, V44A, K47R, N49D, N49S, M53T, G54R, K56E, K56R, T57A, F59S, I61T, E63G, K64R, N65A, N65D, N65F, N65S, L68S, K69E, K69R, R71G, I72A, I72T, I72V, N73G, L74N, V75F, R78G, L80P, K81R, E82G, H88Y, F89L, R92G, S93G, S93R, L94A, D96G, A97T, L98S, K99G, K99R, L100P, E102G, Q103R, P104S, E105G, M112T, M112V, V113A, W114R, I115L, I115V, V116I, G117D, V121A, Y122C, Y122D, Y122I, Y122N, A107T, A107V, N108D, K109E, K109R, V110A, D111N, K123E, K123R, A125F, M1261, N127R, N127S, N127Y, H128R, H128Y, H131R, L132P, K133E, L134P, F135L, F135P, F135S, F135V, V136M, T137R, R138G, R1381, I139T, I139V, M140I, M140V, Q141R, D142G, F143L, F143S, E144G, D146G, T147A, F148L, F148S, F149L, P150L, E151G, I152V, D153A, D153G, E155G, K156R, Y157C, Y157R, K158E, K158R, L159P, L160P, E162G, Y163C, V166A, N168D, S168C, D169G, V170A, Q171R, E172G, E173A, E173G, K174R, I176A, I176F, I176T, K177E, K177R, Y178C, Y178H, F180L, E181G, V182A, Y183C, Y183H, E184G, E184R, K185del, K185E, K185R, N186D, N186S, D187G, and D187N.

In one embodiment, the DRD may be derived from an hDHFR protein and include more than one mutation. Any of the mutations listed herein may be included in the DRD. Non-limiting examples of double mutations include C7R, Y163C; A10V, H88Y; I17V, Y122I; G21T, Y122I; Q36K, Y122I; M53T, R1381; T57A, I72A; E63G, I176F; L74N, Y122I; V75F, Y122I; L94A, T147A; V121A, Y122I; Y122I, A125F; Y122I, N127Y; Y122I, M140I; H131R, E144G; T137R, F143L; E162G, I176F; Y178H, E181G; Y183H, K185E; M1del, I17V; M1del, Y122I; M1del, K185E; M1del, N127Y; M1del, N168D; and M1del, M140I. Non-limiting examples of triple mutations include I8V, K133E, Y163C; V9A, S93R, P150L; K19E, F89L, E181G; G21E, I72V, I176T; L23S, V121A, Y157C; E31D, F32M, V116I; Q36F, N65F, Y122I; Q36K, N65F, Y122I; Q36F, Y122I, A125F; N49D, F59S, D153G; V110A, V136M, K177R; Y122I, H131R, E144G; M1del, I17V, Y122I; M1del, G21T, Y122I; M1del, G21T, Y122N; M1del, Q36K, Y122I; M1del, M53T, R1381; M1del, L74N, Y122I; M1del, V75F, Y122I; M1del, L94A, T147A; M1del, V121A, Y122I; M1del, Y122I, A125F; M1del, Y122I, M140I; M1del, Y122I, N127Y; M1del, H131R, E144G; and M1del, E162G, I176F. Non-limiting examples of four mutations include G54R, I115L, M140V, S168C; M1del, E31D, F32M, V116I; M1del, Q36F, N65F, Y122I; M1del, Q36F, Y122I, A125F; M1del, Q36K, N65F, Y122I; and M1del, Y122I, H131R, E144G. Non-limiting examples of five mutations include V2A, R33G, Q36R, L100P, K185R; D22S, F32M, R33S, Q36S, N65S; and M1del, D22S, F32M, R33S, Q36S, N65S. Non-limiting example of more than five mutations include I17N, L98S, K99R, M112T, E151G, E162G, E172G; G16S, I17V, F89L, D96G, K123E, M140V, D146G, K156R; K81R, K99R, L100P, E102G, N108D, K123R, H128R, D142G, F180L, K185E; R138G, D142G, F143S, K156R, K158E, E162G, V166A, K177E, Y178C, K185E, N186S; N14S, P24S, F35L, M53T, K56E, R92G, S93G, N127S, H128Y, F135L, F143S, L159P, L160P, E173A, F180L; F35L, R37G, N65A, L68S, K69E, R71G, L80P, K99G, G117D, L132P, I139V, M140I, D142G, D146G, E173G, D187G; L28P, N30H, M38V, V44A, L68S, N73G, R78G, A97T, K99R, A107T, K109R, D111N, L134P, F135V, T147A, I152V, K158R, E172G, V182A, E184R; V2A, I17V, N30D, E31G, Q36R, F59S, K69E, I72T, H88Y, F89L, N108D, K109E, V110A, I115V, Y122D, L132P, F135S, M140V, E144G, T147A, Y157C, V170A, K174R, N186S; L100P, E102G, Q103R, P104S, E105G, N108D, V113A, W114R, Y122C, M1261, N127R, H128Y, L132P, F135P, I139T, F148S, F149L, I152V, D153A, D169G, V170A, I176A, K177R, V182A, K185R, N186S; and A10T, Q13R, N14S, N20D, P24S, N30S, M38T, T40A, K47R, N49S, K56R, I61T, K64R, K69R, I72A, R78G, E82G, F89L, D96G, N108D, M112V, W114R, Y122D, K123E, I139V, Q141R, D142G, F148L, E151G, E155G, Y157R, Q171R, Y183C, E184G, K185del, D187N.

In some embodiments, DRDs derived from hDHFR may comprise amino acids 2-187 of the parent hDHFR sequence. This is referred to herein as an M1del mutation.

In one embodiment, the stimulus is a small molecule that binds to a SRE to post-translationally regulate protein levels. In one aspect, DHFR ligands: trimethoprim (TMP) and methotrexate (MTX) are used to stabilize hDHFR mutants.

A non-limiting listing of hDHFR derived drug responsive domains (amino acid and nucleic acid sequences) are listed in Table 2. The position of the mutated amino acid listed in Table 2 is relative to human DHFR (Uniprot ID: P00374) of SEQ ID NO: 30 for DRDs derived from hDHFR (referred to in Table 2 as the “WT”). In Table 2, “del” means that the mutation is the deletion of the amino acid at that position relative to the wild type sequence. In Table 2, a DHFR derived drug responsive domain comprising amino acids 1-187 of the parent hDHFR sequence is denoted as hDHFR with the identity of the mutations within parenthesis e.g. hDHFR (Y122I).

TABLE 2
hDHFR Derived Drug responsive domains (DDs)

| | | Amino acid | Nucleic Acid |
|---|---|---|---|
| DRD Identifier | hDHFR Regions and Mutations | SEQ ID | SEQ ID |
| hDHFRDD-1 | hDHFR (Y122I) | 78 | 79 |
| hDHFRDD-2 | hDHFR (K81R) | 80 | 81 |
| hDHFRDD-3 | hDHFR (F59S) | 82 | 83 |
| hDHFRDD-4 | hDHFR (I17V) | 84 | 85 |
| hDHFRDD-5 | hDHFR (N65D) | 86 | 87 |
| hDHFRDD-6 | hDHFR (A107V) | 88 | 89 |
| hDHFRDD-7 | hDHFR (N127Y) | 90 | 91 |
| hDHFRDD-8 | hDHFR (M140I) | 92 | 93 |
| hDHFRDD-9 | hDHFR (K185E) | 94 | 95 |
| hDHFRDD-10 | hDHFR (N186D) | 96 | 97 |
| hDHFRDD-11 | hDHFR (2-187 of WT) | 98 | 99 |
| hDHFRDD-12 | hDHFR (2-187 of WT, N168D) | 100 | 101 |
| hDHFRDD-13 | hDHFR (2-187 of WT, M140I) | 102 | 103 |
| hDHFRDD-14 | hDHFR (C7R, Y163C) | 104 | 105 |
| hDHFRDD-15 | hDHFR (A10V, H88Y) | 106 | 107 |
| hDHFRDD-16 | hDHFR (I17V, Y122I) | 108 | 109 |
| hDHFRDD-17 | hDHFR (G21T, Y122I) | 110 | 111 |
| hDHFRDD-18 | hDHFR (Q36K, Y122I) | 112 | 113 |
| hDHFRDD-19 | hDHFR (M53T, R138I) | 114 | 115 |
| hDHFRDD-20 | hDHFR (T57A, I72A) | 116 | 117 |
| hDHFRDD-21 | hDHFR (E63G, I176F) | 118 | — |
| hDHFRDD-22 | hDHFR (L74N, Y122I) | 119 | 120 |
| hDHFRDD-23 | hDHFR(V75F, Y122I) | 121 | 122 |
| hDHFRDD-24 | hDHFR(L94A, T147A) | 123 | 124 |
| hDHFRDD-25 | hDHFR(V121A, Y122I) | 125 | 126 |
| hDHFRDD-26 | hDHFR(Y122I, A125F) | 127 | 128 |
| hDHFRDD-27 | hDHFR(Y122I, N127Y) | 129 | 130 |
| hDHFRDD-28 | hDHFR(Y122I, M140I) | 131 | 132 |
| hDHFRDD-29 | hDHFR(H131R, E144G) | 133 | 134 |
| hDHFRDD-30 | hDHFR(T137R, F143L) | 135 | 136 |
| hDHFRDD-31 | hDHFR(E162G, I176F) | 137 | 138 |
| hDHFRDD-32 | hDHFR(Y178H, E181G) | 139 | 140 |
| hDHFRDD-33 | hDHFR(Y183H, K185E) | 141 | 142 |
| hDHFRDD-34 | hDHFR(2-187 of WT, I17V) | 143 | 144 |
| hDHFRDD-35 | hDHFR(2-187 of WT, Y122I) | 145 | 146-148 |
| hDHFRDD-36 | hDHFR(2-187 of WT, K185E) | 149 | 150 |
| hDHFRDD-37 | hDHFR(2-187 of WT, N127Y) | 151 | 152 |
| hDHFRDD-38 | hDHFR(I8V, K133E, Y163C) | 153 | 154 |
| hDHFRDD-39 | hDHFR(V9A, S93R, P150L) | 155 | 156 |
| hDHFRDD-40 | hDHFR(K19E, F89L, E181G) | 157 | 158 |
| hDHFRDD-41 | hDHFR(G21E, I72V, I176T) | 159 | 160 |
| hDHFRDD-42 | hDHFR(L23S, V121A, Y157C) | 161 | 162 |
| hDHFRDD-43 | hDHFR(E31D, F32M, V116I) | 163 | 164 |
| hDHFRDD-44 | hDHFR(Q36F, N65F, Y122I) | 165 | — |
| hDHFRDD-45 | hDHFR(Q36K, N65F, Y122I) | 166 | 167 |
| hDHFRDD-46 | hDHFR(Q36F, Y122I, A125F) | 168 | 169 |
| hDHFRDD-47 | hDHFR(N49D, F59S, D153G) | 170 | 171 |
| hDHFRDD-48 | hDHFR(V110A, V136M, K177R) | 172 | 173 |
| hDHFRDD-49 | hDHFR(Y122I, H131R, E144G) | 174 | 175 |
| hDHFRDD-50 | hDHFR(2-187 of WT, I17V, Y122I) | 176 | 177-178 |
| hDHFRDD-51 | hDHFR(2-187 of WT, G21T, Y122I) | 179 | 180 |
| hDHFRDD-52 | hDHFR(2-187 of WT, G21T, Y122N) | 181 | 182 |
| hDHFRDD-53 | hDHFR(2-187 of WT, Q36K, Y122I) | 183 | 184-186 |
| hDHFRDD-54 | hDHFR(2-187 of WT, M53T, R138I) | 187 | 188 |
| hDHFRDD-55 | hDHFR(2-187 of WT, L74N, Y122I) | 189 | 190 |
| hDHFRDD-56 | hDHFR(2-187 of WT, V75F, Y122I) | 191 | 192 |
| hDHFRDD-57 | hDHFR(2-187 of WT, L94A, T147A) | 193 | 194 |
| hDHFRDD-58 | hDHFR(2-187 of WT, V121A, Y122I) | 195 | 196 |
| hDHFRDD-59 | hDHFR(2-187 of WT, Y122I, A125F) | 197 | 198-200 |
| hDHFRDD-60 | hDHFR(2-187 of WT, Y122I, M140I) | 201 | 202 |
| hDHFRDD-61 | hDHFR(2-187 of WT, Y122I, N127Y) | 203 | 204 |
| hDHFRDD-62 | hDHFR(2-187 of WT, H131R, E144G) | 205 | 206 |
| hDHFRDD-63 | hDHFR(2-187 of WT, E162G, I176F) | 207 | 208 |
| hDHFRDD-64 | hDHFR(G54R, M140V, S168C) | 209 | — |
| hDHFRDD-65 | hDHFR(G54R, I115L, M140V, S168C) | 210 | 211 |
| hDHFRDD-66 | hDHFR(V2A, R33G, Q36R, L100P, | 212 | 213 |
| | K185R) | | |
| hDHFRDD-67 | hDHFR(D22S, F32M, R33S, Q36S, | 214 | 215 |
| | N65S) | | |
| hDHFRDD-68 | hDHFR(2-187 of WT, E31D, F32M, | 216 | 217 |
| | V116I) | | |
| hDHFRDD-69 | hDHFR(2-187 of WT, Q36F, N65F, | 218 | 219-220 |
| | Y122I) | | |
| hDHFRDD-70 | hDHFR(2-187 of WT, Q36F, Y122I, | 221 | 222 |
| | A125F) | | |
| hDHFRDD-71 | hDHFR(2-187 of WT, Q36K, N65F, | 223 | 224 |
| | Y122I) | | |
| hDHFRDD-72 | hDHFR(2-187 of WT, Y122I, H131R, | 225 | 226 |
| | E144G) | | |
| hDHFRDD-73 | hDHFR(2-187 of WT, D22S, F32M, | 227 | 228 |
| | R33S, Q36S, N65S) | | |
| hDHFRDD-74 | hDHFR(I17N, L98S, K99R, M112T, | 229 | 230 |
| | E151G, E162G, E172G) | | |
| hDHFRDD-75 | hDHFR(G16S, I17V, F89L, D96G, | 231 | 232 |
| | K123E, M140V, D146G, K156R) | | |
| hDHFRDD-76 | hDHFR(K81R, K99R, L100P, E102G, | 233 | 234 |
| | N108D, K123R, H128R, D142G, F180L, | | |
| | K185E) | | |
| hDHFRDD-77 | hDHFR(R138G, D142G, F143S, K156R, | 235 | 236 |
| | K158E, E162G, V166A, K177E, Y178C, | | |
| | K185E, N186S) | | |
| hDHFRDD-78 | hDHFR(N14S, P24S, F35L, M53T, | 237 | 238 |
| | K56E, R92G, S93G, N127S, H128Y, | | |
| | F135L, F143S, L159P, L160P, E173A, | | |
| | F180L) | | |
| hDHFRDD-79 | hDHFR(F35L, R37G, N65A, L68S, | 239 | 240 |
| | K69E, R71G, L80P, K99G, G117D, | | |
| | L132P, I139V, M140I, D142G, D146G, | | |
| | E173G, D187G) | | |
| hDHFRDD-80 | hDHFR(L28P, N30H, M38V, V44A, | 241 | 242 |
| | L68S, N73G, R78G, A97T, K99R, | | |
| | A107T, K109R, D111N, L134P, F135V, | | |
| | T147A, I152V, K158R, E172G, V182A, | | |
| | E184R) | | |
| hDHFRDD-81 | hDHFR(V2A, I17V, N30D, E31G, Q36R, | 243 | 244 |
| | F59S, K69E, I72T, H88Y, F89L, N108D, | | |
| | K109E, V110A, I115V, Y122D, L132P, | | |
| | F135S, M140V, E144G, T147A, Y157C, | | |
| | V170A, K174R, N186S) | | |
| hDHFRDD-82 | hDHFR(L100P, E102G, Q103R, P104S, | 245 | 246 |
| | E105G, N108D, V113A, W114R, Y122C, | | |
| | M126I, N127R, H128Y, L132P, F135P, | | |
| | I139T, F148S, F149L, I152V, D153A, | | |
| | D169G, V170A, I176A, K177R, V182A, | | |
| | K185R, N186S) | | |
| hDHFRDD-83 | hDHFR(A10T, Q13R, N14S, N20D, | 247 | 248 |
| | P24S, N30S, M38T, T40A, K47R, N49S, | | |
| | K56R, I61T, K64R, K69R, I72A, R78G, | | |
| | E82G, F89L, D96G, N108D, M112V, | | |
| | W114R, Y122D, K123E, I139V, Q141R, | | |
| | D142G, F148L, E151G, E155G, Y157R, | | |
| | Q171R, Y183C, E184G, K185del, | | |
| | D187N) | | |
| hDHFRDD-84 | hDHFR(2-187 of WT, I17A) | 249 | 250 |
| hDHFRDD-85 | hDHFR(2-187 of WT, I17A, Y122I) | 251 | 252 |
| hDHFRDD-86 | hDHFR (aa 2-187 of WT, K55R, N65K, | 6552 | 6553 |
| | Y122I) | | |

In one embodiment, the SRE may include at least one hDHFR-derived drug responsive domain (DD) such as, but not limited to, hDHFRDD-1, hDHFRDD-2, hDHFRDD-3, hDHFRDD-4, hDHFRDD-5, hDHFRDD-6, hDHFRDD-7, hDHFRDD-8, hDHFRDD-9, hDHFRDD-10, hDHFRDD-11, hDHFRDD-12, hDHFRDD-13, hDHFRDD-14, hDHFRDD-15, hDHFRDD-16, hDHFRDD-17, hDHFRDD-18, hDHFRDD-19, hDHFRDD-20, hDHFRDD-21, hDHFRDD-22, hDHFRDD-23, hDHFRDD-24, hDHFRDD-25, hDHFRDD-26, hDHFRDD-27, hDHFRDD-28, hDHFRDD-29, hDHFRDD-30, hDHFRDD-31, hDHFRDD-32, hDHFRDD-33, hDHFRDD-34, hDHFRDD-35, hDHFRDD-36, hDHFRDD-37, hDHFRDD-38, hDHFRDD-39, hDHFRDD-40, hDHFRDD-41, hDHFRDD-42, hDHFRDD-43, hDHFRDD-44, hDHFRDD-45, hDHFRDD-46, hDHFRDD-47, hDHFRDD-48, hDHFRDD-49, hDHFRDD-50, hDHFRDD-51, hDHFRDD-52, hDHFRDD-53, hDHFRDD-54, hDHFRDD-55, hDHFRDD-56, hDHFRDD-57, hDHFRDD-58, hDHFRDD-59, hDHFRDD-60, hDHFRDD-61, hDHFRDD-62, hDHFRDD-63, hDHFRDD-64, hDHFRDD-65, hDHFRDD-66, hDHFRDD-67, hDHFRDD-68, hDHFRDD-69, hDHFRDD-70, hDHFRDD-71, hDHFRDD-72, hDHFRDD-73, hDHFRDD-74, hDHFRDD-75, hDHFRDD-76, hDHFRDD-77, hDHFRDD-78, hDHFRDD-79, hDHFRDD-80, hDHFRDD-81, hDHFRDD-82, hDHFRDD-83, hDHFRDD-84, hDHFRDD-85 and hDHFRDD-86.

6. E. coli Dihydrofolate Reductase (ecDHFR) Derived Drug Responsive Domains (DRDs)

In one embodiment, the SRE may include at least one drug responsive domain (DRD) derived from an E. coli dihydrofolate reductase (ecDHFR) protein or a fragment or variant thereof.

In one embodiment, the SRE comprises a region of the ecDHFR protein. The region of the ecDHFR protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the DRD may be derived from an ecDHFR protein and include at least one mutation. Non-limiting examples of mutations include M1del, R12Y, R12H, G27S, Y100I, and E129K.

In one embodiment, the DRD may be derived from an ecDHFR protein and include more than one mutation. Any of the mutations listed herein may be included in the DRD. Non-limiting examples of double mutations include R12Y and Y100I. Non-limiting examples of triple mutations include M1del, R12Y, Y100I; M1del, R12Y, E129K; and M1del, R12H, E129K. Non-limiting examples of four mutations include M1del, R12Y, G27S, Y100I.

In some embodiments, DRDs derived from ecDHFR may comprise amino acids 2-159 of the parent ecDHFR sequence. This is referred to herein as an M1del mutation.

In one embodiment, the stimulus is a small molecule that binds to a SRE to post-translationally regulate protein levels. In one aspect, ecDHFR ligands: trimethoprim (TMP) and methotrexate (MTX) are used to stabilize ecDHFR mutants.

A non-limiting listing of ecDHFR derived drug responsive domains (amino acid and nucleic acid sequences) are listed in Table 3. The position of the mutated amino acid listed in Table 3 is relative to E. coli DHFR (Uniprot ID: P0ABQ4) of SEQ ID NO: 35 (referred to Table 3 as the “WT”. In Table 3, “del” means that the mutation is the deletion of the amino acid at that position relative to the wild type sequence. In Table 3, a ecDHFR derived drug responsive domain comprising amino acids 1-159 of the parent ecDHFR sequence is denoted as ecDHFR with the identity of the mutations within parenthesis e.g. ecDHFR (R12Y, Y100I).

TABLE 3
ecDHFR Derived Drug responsive domains (DDs)

| | | Amino | Nucleic |
|---|---|---|---|
| | | acid SEQ | Acid |
| DRD Identifier | ecDHFR Mutations | ID | SEQ ID |
| ecDHFRDD-1 | ecDHFR (R12Y, Y100I) | 253 | 254 |
| ecDHFRDD-2 | ecDHFR (aa 2-159 of WT, | 255 | 256-263 |
| | R12Y, Y100I) | | |
| ecDHFRDD-3 | ecDHFR (aa 2-159 of WT, | 264 | 265-266 |
| | R12H, E129K) | | |
| ecDHFRDD-4 | ecDHFR (aa 2-159 of WT, | 267 | 268 |
| | R12Y, E129K) | | |
| ecDHFRDD-5 | ecDHFR (aa 2-159 of WT, | 269 | — |
| | R12Y, G27S, Y100I) | | |
| ecDHFRDD-6 | ecDHFR (aa 2-159 of WT, | 6554 | 6555 |
| | Y100A) | | |
| ecDHFRDD-7 | ecDHFR (aa 2-159 of WT, | 6556 | 6557 |
| | Y100C) | | |
| ecDHFRDD-8 | ecDHFR (aa 2-159 of WT, | 6558 | 6559 |
| | Y100D) | | |
| ecDHFRDD-9 | ecDHFR (aa 2-159 of WT, | 6560 | 6561 |
| | Y100E) | | |
| ecDHFRDD-10 | ecDHFR (aa 2-159 of WT, | 6562 | 6563 |
| | Y100F) | | |
| ecDHFRDD-11 | ecDHFR (aa 2-159 of WT, | 6564 | 6565 |
| | Y100G) | | |
| ecDHFRDD-12 | ecDHFR (aa 2-159 of WT, | 6566 | 6567 |
| | Y100H) | | |
| ecDHFRDD-13 | ecDHFR (aa 2-159 of WT, | 6568 | 6569 |
| | Y100I) | | |
| ecDHFRDD-14 | ecDHFR (aa 2-159 of WT, | 6570 | 6571 |
| | Y100K) | | |
| ecDHFRDD-15 | ecDHFR (aa 2-159 of WT, | 6572 | 6573 |
| | Y100L) | | |
| ecDHFRDD-16 | ecDHFR (aa 2-159 of WT, | 6574 | 6575 |
| | Y100M) | | |
| ecDHFRDD-17 | ecDHFR (aa 2-159 of WT, | 6576 | 6577 |
| | Y100N) | | |
| ecDHFRDD-18 | ecDHFR (aa 2-159 of WT, | 6578 | 6579 |
| | Y100P) | | |
| ecDHFRDD-19 | ecDHFR (aa 2-159 of WT, | 6580 | 6581 |
| | Y100Q) | | |
| ecDHFRDD-20 | ecDHFR (aa 2-159 of WT, | 6582 | 6583 |
| | Y100R) | | |
| ecDHFRDD-21 | ecDHFR (aa 2-159 of WT, | 6584 | 6585 |
| | Y100S) | | |
| ecDHFRDD-22 | ecDHFR (aa 2-159 of WT, | 6586 | 6587 |
| | Y100T) | | |
| ecDHFRDD-23 | ecDHFR (aa 2-159 of WT, | 6588 | 6589 |
| | Y100V) | | |
| ecDHFRDD-24 | ecDHFR (aa 2-159 of WT, | 6590 | 6591 |
| | Y100W) | | |
| ecDHFRDD-25 | ecDHFR (aa 2-159 of WT) | 6592 | 6593 |

In one embodiment, the SRE may include at least one ecDHFR-derived drug responsive domain (DD) such as, but not limited to, ecDHFRDD-1, ecDHFRDD-2, ecDHFRDD-3, ecDHFRDD-4, ecDHFRDD-5, ecDHFRDD-6, ecDHFRDD-7, ecDHFRDD-8, ecDHFRDD-9, ecDHFRDD-10, ecDHFRDD-11, ecDHFRDD-12, ecDHFRDD-13, ecDHFRDD-14, ecDHFRDD-15, ecDHFRDD-16, ecDHFRDD-17, ecDHFRDD-18, ecDHFRDD-19, ecDHFRDD-20, ecDHFRDD-21, ecDHFRDD-22, ecDHFRDD-23, ecDHFRDD-24, and ecDHFRDD-25.

FK506 Binding Protein (FKBP) Derived Drug Responsive Domains (DRDs)

In one embodiment, the SRE may include at least one drug responsive domain (DRD) derived from a FK506 binding protein (FKBP) protein or a fragment or variant thereof.

In one embodiment, the SRE comprises a region of the FKBP protein. The region of the FKBP protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the DRD may be derived from a FKBP protein and include at least one mutation. Non-limiting examples of mutations include M1del, E32G, F37V, R72G, K106E, and L109P.

In one embodiment, the DRD may be derived from a FKBP protein and include more than one mutation. Any of the mutations listed herein may be included in the DD. Non-limiting examples of double mutations include M1del, F37V; F37V, L107P. A non-limiting example of triple mutations include M1del, F37V, L107P. A non-limiting example of four mutations include E32G, F37V, R72G, K106E. A non-limiting example of five mutations include M1del, E32G, F37V, R72G, K106E.

In some embodiments, DRDs derived from FKBP may comprise amino acids 2-108 of the parent FKBP sequence. This is referred to herein as an M1del mutation.

In one embodiment, the stimulus is a small molecule that binds to a SRE to post-translationally regulate protein levels. In one aspect, FKBP ligand Shield-1 is used to stabilize FKBP mutants.

A non-limiting listing of FKBP derived drug responsive domains (amino acid and nucleic acid sequences) are listed in Table 4. The position of the mutated amino acid listed in Table 4 is relative to FKBP (Uniprot ID: P62942) of SEQ ID NO: 37 (referred to in Table 4 as the “WT”). In Table 4, “del” means that the mutation is the deletion of the amino acid at that position relative to the wild type sequence. In Table 4, a FKBP derived drug responsive domain comprising amino acids 1-108 of the parent FKBP sequence is denoted as FKBP with the identity of the mutations within parenthesis e.g. FKBP (F37V).

TABLE 4
FKBP Derived Drug responsive domains (DRDs)

| DRD | | Amino acid | Nucleic Acid |
|---|---|---|---|
| Identifier | FKBP Mutations | SEQ ID | SEQ ID |
| FKBPDD-1 | FKBP (2-108 of WT) | 270 | |
| FKBPDD-2 | FKBP (F37V) | 271 | |
| FKBPDD-3 | FKBP (2-108 of WT, F37V) | 272 | 273 |
| FKBPDD-4 | FKBP(F37V, L107P) | 274 | 275-276 |
| FKBPDD-5 | FKBP(2-108 of WT, F37V, | 277 | 278-284 |
| | L107P) | | |
| FKBPDD-6 | FKBP(E32G, F37V, R72G, | 285 | |
| | K106E) | | |
| FKBPDD-7 | FKBP(2-108 of WT, E32G, | 286 | 287-293 |
| | F37V, R72G, K106E) | | |

In one embodiment, the SRE may include at least one FKBP-derived drug responsive domain (DD) such as, but not limited to, FKBPDD-1, FKBPDD-2, FKBPDD-3, FKBPDD-4, FKBPDD-5, FKBPDD-6, and FKBPDD-7.

7. Human Phosphodiesterase (hPDE) Derived Drug Responsive Domains (DRDs)

In one embodiment, the SRE may include at least one drug responsive domain (DRD) derived from a human phosphodiesterase (hPDE) protein such as, but not limited to, human phosphodiesterase 1A (hPDE1A), human phosphodiesterase 1B (hPDE1B), human phosphodiesterase 1C (hPDE1C), human phosphodiesterase 1D (hPDE1D), human phosphodiesterase 2A (hPDE2A), human phosphodiesterase 3A (hPDE3A), human phosphodiesterase 3B (hPDE3B), human phosphodiesterase 4A (hPDE4A), human phosphodiesterase 4B (hPDE4B), human phosphodiesterase 4C (hPDE4C), human phosphodiesterase 4D (hPDE4D), human phosphodiesterase 6A (hPDE6A), human phosphodiesterase 6B (hPDE6B), human phosphodiesterase 6C (hPDE6C), human phosphodiesterase 7A (hPDE7A), human phosphodiesterase 7B (hPDE7B), human phosphodiesterase 8A (hPDE8A), human phosphodiesterase 8B (hPDE8B), human phosphodiesterase 9A (hPDE9A), human phosphodiesterase 10A (hPDE10A), and human phosphodiesterase 11A (hPDE11A), or a fragment or variant thereof. As a non-limiting example, the SRE comprises at least one DD derived from hPDE1A. As a non-limiting example, the SRE comprises at least one DD derived from hPDE1B. As a non-limiting example, the SRE comprises at least one DD derived from hPDE1C. As a non-limiting example, the SRE comprises at least one DD derived from hPDE1D. As a non-limiting example, the SRE comprises at least one DD derived from hPDE2A. As a non-limiting example, the SRE comprises at least one DD derived from hPDE3A. As a non-limiting example, the SRE comprises at least one DD derived from h hPDE3B. As a non-limiting example, the SRE comprises at least one DD derived from hPDE4A. As a non-limiting example, the SRE comprises at least one DD derived from hPDE4B. As a non-limiting example, the SRE comprises at least one DD derived from hPDE4C. As a non-limiting example, the SRE comprises at least one DD derived from hPDE4D. As a non-limiting example, the SRE comprises at least one DD derived from hPDE6A. As a non-limiting example, the SRE comprises at least one DD derived from hPDE6B. As a non-limiting example, the SRE comprises at least one DD derived from hPDE6C. As a non-limiting example, the SRE comprises at least one DD derived from hPDE7A. As a non-limiting example, the SRE comprises at least one DD derived from hPDE7B. As a non-limiting example, the SRE comprises at least one DD derived from hPDE8A. As a non-limiting example, the SRE comprises at least one DD derived from hPDE8B. As a non-limiting example, the SRE comprises at least one DD derived from hPDE9A. As a non-limiting example, the SRE comprises at least one DD derived from hPDE10A. As a non-limiting example, the SRE comprises at least one DD derived from hPDE11A.

In one embodiment, the SRE comprises a region of the hPDE protein. The region of the hPDE protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the DRD may be derived from a PDE protein and include at least one mutation.

In one embodiment, the DRD may be derived from a PDE5 protein and include at least one mutation. Non-limiting examples of mutations include E535D, E536G, Q541R, S542L, V548M, P549S, S550F, L554R, K555R, I556S, F559L, S560G, F561L, S562G, F564L, F564S, F564I, L569M, T571S, T571I, L573M, C574Y, V585A, V585M, N587S, Q586L, Q586P, Q589E, K591E, H592R, V594I, I599V, K604R, K604E, N605D, N605Y, R607W, R607Q, K608E, N609H, N609Y, A611T, Y612F, Y612W, Y612A, H613L, H613R, W615R, H617L, N620S, M625I, K630R, K633E, N636S, I648V, H653A, D656L, R658H, G659A, N661S, N662Y, S663Y, S663P, Q666H, L675P, Y676D, Y676N, C677R, H678R, I680S, E682A, D687A, H685L, M691I, L693P, I700F, I706N, E708V, Y709H, K710N, T711A, T712S, T712M, I715K, A722V, T723S, T723R, D724Y, D724A, D724N, D724G, L727P, Y728L, K730E, R732L, R732G, R732A, R732V, R7321, R732P, R732F, R732W, R732Y, R732H, R732S, R732T, R732D, R732E, R732Q, R732N, R732M, R732C, R732K, F735L, F736A, F736G, F736L, F736M, F736R, F736W, F736K, F736Q, F736E, F736S, F736P, F736V, F736C, F736Y, F736H, F736I, F736N, F736D, F736T, L738H, N742S, Q743L, F744L, L746S, F755L, F755Y, M758I, M7601, A762T, A762S, D764N, D764G, D764V, D764A, S766F, A767E, I768N, K770Q, W772C, A779T, L781P, T784I, F787V, F787A, K795E, E795R, K795N, E796G, E796D, L797F, I799L, I799T, T802P, D803N, L804P, E808G, S815C, M816A, M816T, F820I, I821A, A823T, I824T, Y829A, T833S, C839S, F840S, R847G, R847T, K848N, K852E, W853F, E858V, and Q859R.

In one embodiment, the DRD may be derived from a PDE5 protein and include more than one mutation. Any of the mutations listed herein may be included in the DD. Non-limiting examples of double mutations include Y612A, R732L; Y612F, R732L; Y612W, R732L; Y709H, F787V; Y728L, D764N; L569M, T833S; D724A, R732L; D724G, D764G; D724G, K848N; E682A, R732L; F736A, D764N; G659A, T784I; H617L, A722V; H653A, R732L; I556S, E796D; I700F, E796G; K770Q, K848N; L573M, F735L; N605Y, I715K; N609Y, I799L; R732L, D764A; R732L, D764N; R732L, F736A; S550F, L554R; V548M, D803N; V548M, F820I. A non-limiting example of triple mutations include A722V, F755L, M7601; F561L, G659A, T784I; H613L, D724Y, F755Y; L554R, Q589E, A823T; L554R, Q589E, M691I; S542L, E708V, W772C; S562G, L727P, R847T; T571S, V585M, T723S; T712M, M758I, Q859R. A non-limiting example of four mutations include L554R, Q586P, K710N, K730E; Q586L, S663Y, A762T, E808G; T571I, K604R, I706N, E795R; W615R, T723R, A762T, E808G. A non-limiting example of five mutations include F564I, N662Y, H685L, L693P, F736I; P549S, F564I, R658H, A779T, R847G.

In some embodiments, DRDs derived from PDE5 may comprise amino acids 2-875 of the parent PDE5 sequence. This is referred to herein as an M1del mutation.

In some embodiments, DRDs are derived from a region of the PDE5 protein. As a non-limiting example, the region is amino acid 535-860 of hPDE5 (SEQ ID NO: 43). As a non-limiting example, the region is amino acid 535-830 of hPDE5 (SEQ ID NO: 44). As a non-limiting example, the region is amino acid 535-836 of hPDE5 (SEQ ID NO: 45). As a non-limiting example, the region is amino acid 567-860 of hPDE5 (SEQ ID NO: 46). As a non-limiting example, the region is amino acid 590-836 of hPDE5 (SEQ ID NO: 47). As a non-limiting example, the region is amino acid 590-860 of hPDE5 (SEQ ID NO: 48).

In one embodiment, the stimulus is a small molecule that binds to a SRE to post-translationally regulate protein levels. In one aspect, PDE5 ligands Sildenafil, Vardenafil, or Tadalafil are used to stabilize PDE5 mutants.

A non-limiting listing of PDE5 derived drug responsive domains (amino acid and nucleic acid sequences) are listed in Table 5. The position of the mutated amino acid listed in Table 5 is relative to PDE5 (Uniprot ID: 076074) of SEQ ID NO: 1. In Table 5, “del” means that the mutation is the deletion of the amino acid at that position relative to the wild type sequence.

TABLE 5
PDE5 Derived Drug responsive domains (DRDs)

| | | Amino | Nucleic |
|---|---|---|---|
| DRD | | acid | Acid |
| Identifier | PDE5 region and Mutations) | SEQ ID | SEQ ID |
| PDE5DD-1 | PDE5 (aa 535-860 of WT, W853F) | 294 | 295 |
| PDE5DD-2 | PDE5 (aa 535-860 of WT, I821A) | 296 | 297 |
| PDE5DD-3 | PDE5 (aa 535-860 of WT, Y829A) | 298 | 299 |
| PDE5DD-4 | PDE5 (aa 535-860 of WT, F787A) | 300 | 301 |
| PDE5DD-5 | PDE5 (aa 535-860 of WT, F736A) | 302 | 303-305 |
| PDE5DD-6 | PDE5 (aa 535-860 of WT, Y728L) | 306 | 307 |
| PDE5DD-7 | PDE5 (aa 535-860 of WT, R732L) | 308 | 309-312 |
| PDE5DD-8 | PDE5 (aa 535-860 of WT, M625I) | 313 | 314 |
| PDE5DD-9 | PDE5 (aa 535-860 of WT, D656L) | 315 | 316 |
| PDE5DD-10 | PDE5 (aa 535-860 of WT, E535D) | 317 | — |
| PDE5DD-11 | PDE5 (aa 535-860 of WT, E536G) | 318 | — |
| PDE5DD-12 | PDE5 (aa 535-860 of WT, Q541R) | 319 | — |
| PDE5DD-13 | PDE5 (aa 535-860 of WT, K555R) | 320 | — |
| PDE5DD-14 | PDE5 (aa 535-860 of WT, F559L) | 321 | — |
| PDE5DD-15 | PDE5 (aa 535-860 of WT, F561L) | 322 | — |
| PDE5DD-16 | PDE5 (aa 535-860 of WT, F564L) | 323 | — |
| PDE5DD-17 | PDE5 (aa 535-860 of WT, F564S) | 324 | — |
| PDE5DD-18 | PDE5 (aa 535-860 of WT, K591E) | 325 | — |
| PDE5DD-19 | PDE5 (aa 535-860 of WT, N587S) | 326 | — |
| PDE5DD-20 | PDE5 (aa 535-860 of WT, K604E) | 327 | — |
| PDE5DD-21 | PDE5 (aa 535-860 of WT, K608E) | 328 | — |
| PDE5DD-22 | PDE5 (aa 535-860 of WT, N609H) | 329 | — |
| PDE5DD-23 | PDE5 (aa 535-860 of WT, K630R) | 330 | — |
| PDE5DD-24 | PDE5 (aa 535-860 of WT, K633E) | 331 | — |
| PDE5DD-25 | PDE5 (aa 535-860 of WT, N636S) | 332 | — |
| PDE5DD-26 | PDE5 (aa 535-860 of WT, N661S) | 333 | 334 |
| PDE5DD-27 | PDE5 (aa 535-860 of WT, Y676D) | 335 | — |
| PDE5DD-28 | PDE5 (aa 535-860 of WT, Y676N) | 336 | — |
| PDE5DD-29 | PDE5 (aa 535-860 of WT, C677R) | 337 | — |
| PDE5DD-30 | PDE5 (aa 535-860 of WT, H678R) | 338 | — |
| PDE5DD-31 | PDE5 (aa 535-860 of WT, D687A) | 339 | — |
| PDE5DD-32 | PDE5 (aa 535-860 of WT, T712S) | 340 | — |
| PDE5DD-33 | PDE5 (aa 535-860 of WT, D724N) | 341 | — |
| PDE5DD-34 | PDE5 (aa 535-860 of WT, D724G) | 342 | — |
| PDE5DD-35 | PDE5 (aa 535-860 of WT, L738H) | 343 | — |
| PDE5DD-36 | PDE5 (aa 535-860 of WT, N742S) | 344 | — |
| PDE5DD-37 | PDE5 (aa 535-860 of WT, A762S) | 345 | — |
| PDE5DD-38 | PDE5 (aa 535-860 of WT, D764N) | 346 | — |
| PDE5DD-39 | PDE5 (aa 535-860 of WT, D764G) | 347 | — |
| PDE5DD-40 | PDE5 (aa 535-860 of WT, D764V) | 348 | — |
| PDE5DD-41 | PDE5 (aa 535-860 of WT, S766F) | 349 | — |
| PDE5DD-42 | PDE5 (aa 535-860 of WT, K795E) | 350 | — |
| PDE5DD-43 | PDE5 (aa 535-860 of WT, L797F) | 351 | — |
| PDE5DD-44 | PDE5 (aa 535-860 of WT, I799T) | 352 | — |
| PDE5DD-45 | PDE5 (aa 535-860 of WT, T802P) | 353 | — |
| PDE5DD-46 | PDE5 (aa 535-860 of WT, S815C) | 354 | — |
| PDE5DD-47 | PDE5 (aa 535-860 of WT, M816A) | 355 | — |
| PDE5DD-48 | PDE5 (aa 535-860 of WT, I824T) | 356 | — |
| PDE5DD-49 | PDE5 (aa 535-860 of WT, C839S) | 357 | — |
| PDE5DD-50 | PDE5 (aa 535-860 of WT, K852E) | 358 | — |
| PDE5DD-51 | PDE5 (aa 535-860 of WT, S560G) | 359 | — |
| PDE5DD-52 | PDE5 (aa 535-860 of WT, V585A) | 360 | — |
| PDE5DD-53 | PDE5 (aa 535-860 of WT, I599V) | 361 | — |
| PDE5DD-54 | PDE5 (aa 535-860 of WT, I648V) | 362 | — |
| PDE5DD-55 | PDE5 (aa 535-860 of WT, S663P) | 363 | — |
| PDE5DD-56 | PDE5 (aa 535-860 of WT, L675P) | 364 | — |
| PDE5DD-57 | PDE5 (aa 535-860 of WT, T711A) | 365 | — |
| PDE5DD-58 | PDE5 (aa 535-860 of WT, F744L) | 366 | — |
| PDE5DD-59 | PDE5 (aa 535-860 of WT, L746S) | 367 | — |
| PDE5DD-60 | PDE5 (aa 535-860 of WT, F755L) | 368 | — |
| PDE5DD-61 | PDE5 (aa 535-860 of WT, L804P) | 369 | — |
| PDE5DD-62 | PDE5 (aa 535-860 of WT, M816T) | 370 | — |
| PDE5DD-63 | PDE5 (aa 535-860 of WT, F840S) | 371 | — |
| PDE5DD-64 | PDE5 (aa 535-860 of WT, R732G) | 372 | 373-374 |
| PDE5DD-65 | PDE5 (aa 535-860 of WT, R732A) | 375 | 376-377 |
| PDE5DD-66 | PDE5 (aa 535-860 of WT, R732V) | 378 | 379-380 |
| PDE5DD-67 | PDE5 (aa 535-860 of WT, R732I) | 381 | 382-383 |
| PDE5DD-68 | PDE5 (aa 535-860 of WT, R732P) | 384 | 385-386 |
| PDE5DD-69 | PDE5 (aa 535-860 of WT, R732F) | 387 | 388 |
| PDE5DD-70 | PDE5 (aa 535-860 of WT, R732W) | 389 | 390 |
| PDE5DD-71 | PDE5 (aa 535-860 of WT, R732Y) | 391 | 392-393 |
| PDE5DD-72 | PDE5 (aa 535-860 of WT, R732H) | 394 | 395-396 |
| PDE5DD-73 | PDE5 (aa 535-860 of WT, R732S) | 397 | 398-399 |
| PDE5DD-74 | PDE5 (aa 535-860 of WT, R732T) | 400 | 401-402 |
| PDE5DD-75 | PDE5 (aa 535-860 of WT, R732D) | 403 | 404-405 |
| PDE5DD-76 | PDE5 (aa 535-860 of WT, R732E) | 406 | 407-408 |
| PDE5DD-77 | PDE5 (aa 535-860 of WT, R732Q) | 409 | 410-411 |
| PDE5DD-78 | PDE5 (aa 535-860 of WT, R732N) | 412 | 413-414 |
| PDE5DD-79 | PDE5 (aa 535-860 of WT, R732M) | 415 | 416 |
| PDE5DD-80 | PDE5 (aa 535-860 of WT, R732C) | 417 | 418-419 |
| PDE5DD-81 | PDE5 (aa 535-860 of WT, R732K) | 420 | 421 |
| PDE5DD-82 | PDE5 (aa 535-860 of WT, H653A) | 422 | 423 |
| PDE5DD-83 | PDE5 (aa 535-860 of WT, D764A) | 424 | 425 |
| PDE5DD-84 | PDE5 (aa 535-860 of WT, R658H) | 426 | 427 |
| PDE5DD-85 | PDE5 (aa 535-860 of WT, Q666H) | 428 | 429 |
| PDE5DD-86 | PDE5 (aa 535-860 of WT, L781P) | 430 | 431 |
| PDE5DD-87 | PDE5 (aa 535-860 of WT, A767E) | 432 | 433 |
| PDE5DD-88 | PDE5 (aa 535-860 of WT, Q743L) | 434 | 435 |
| PDE5DD-89 | PDE5 (aa 535-860 of WT, V594I) | 436 | 437 |
| PDE5DD-90 | PDE5 (aa 535-860 of WT, H592R) | 438 | 439 |
| PDE5DD-91 | PDE5 (aa 535-860 of WT, E858V) | 440 | 441 |
| PDE5DD-92 | PDE5 (aa 535-860 of WT, T784I) | 442 | 443 |
| PDE5DD-93 | PDE5 (aa 535-860 of WT, F736G) | 444 | 445 |
| PDE5DD-94 | PDE5 (aa 535-860 of WT, F736L) | 446 | 447 |
| PDE5DD-95 | PDE5 (aa 535-860 of WT, F736M) | 448 | 449 |
| PDE5DD-96 | PDE5 (aa 535-860 of WT, F736R) | 450 | 451 |
| PDE5DD-97 | PDE5 (aa 535-860 of WT, F736W) | 452 | 453 |
| PDE5DD-98 | PDE5 (aa 535-860 of WT, F736K) | 454 | 455 |
| PDE5DD-99 | PDE5 (aa 535-860 of WT, F736Q) | 456 | 457 |
| PDE5DD-100 | PDE5 (aa 535-860 of WT, F736E) | 458 | 459 |
| PDE5DD-101 | PDE5 (aa 535-860 of WT, F736S) | 460 | 461 |
| PDE5DD-102 | PDE5 (aa 535-860 of WT, F736P) | 462 | 463 |
| PDE5DD-103 | PDE5 (aa 535-860 of WT, F736V) | 464 | 465 |
| PDE5DD-104 | PDE5 (aa 535-860 of WT, F736I) | 466 | 467 |
| PDE5DD-105 | PDE5 (aa 535-860 of WT, F736C) | 468 | 469 |
| PDE5DD-106 | PDE5 (aa 535-860 of WT, F736Y) | 470 | 471 |
| PDE5DD-107 | PDE5 (aa 535-860 of WT, F736H) | 472 | 473 |
| PDE5DD-108 | PDE5 (aa 535-860 of WT, F736N) | 474 | 475 |
| PDE5DD-109 | PDE5 (aa 535-860 of WT, F736D) | 476 | 477 |
| PDE5DD-110 | PDE5 (aa 535-860 of WT, F736T) | 478 | 479 |
| PDE5DD-111 | PDE5 (aa 535-860 of WT, I680S) | 480 | 481 |
| PDE5DD-112 | PDE5 (aa 535-860 of WT, A611T) | 482 | 483 |
| PDE5DD-113 | PDE5 (aa 535-860 of WT, I768N) | 484 | 485 |
| PDE5DD-114 | PDE5 (aa 535-860 of WT, R607W) | 486 | 487 |
| PDE5DD-115 | PDE5 (aa 535-860 of WT, N620S) | 488 | 489 |
| PDE5DD-116 | PDE5 (aa 535-860 of WT, C574Y) | 490 | 491 |
| PDE5DD-117 | PDE5 (aa 535-860 of WT, H613R) | 492 | 493 |
| PDE5DD-118 | PDE5 (aa 535-860 of WT, K795N) | 494 | 495 |
| PDE5DD-119 | PDE5 (aa 535-860 of WT, N605D) | 496 | 497 |
| PDE5DD-120 | PDE5 (aa 535-860 of WT, I799L) | 498 | 499 |
| PDE5DD-121 | PDE5 (aa 535-860 of WT, R607Q) | 500 | 501 |
| PDE5DD-122 | PDE5 (aa 535-860 of WT, E682A) | 502 | 503 |
| PDE5DD-123 | PDE5 (aa 535-860 of WT, D724A) | 504 | 505 |
| PDE5DD-124 | PDE5 (aa 590-860 of WT, R732L) | 506 | 507 |
| PDE5DD-125 | PDE5 (aa 567-860 of WT, R732L) | 508 | 509 |
| PDE5DD-126 | PDE5 (aa 590-836 of WT, R732G) | 510 | 511 |
| PDE5DD-127 | PDE5 (aa 590-836 of WT, R732A) | 512 | 513 |
| PDE5DD-128 | PDE5 (aa 590-836 of WT, R732V) | 514 | 515 |
| PDE5DD-129 | PDE5 (aa 590-836 of WT, R732I) | 516 | 517 |
| PDE5DD-130 | PDE5 (aa 590-836 of WT, R732P) | 518 | 519 |
| PDE5DD-131 | PDE5 (aa 590-836 of WT, R732F) | 520 | 521 |
| PDE5DD-132 | PDE5 (aa 590-836 of WT, R732W) | 522 | 523 |
| PDE5DD-133 | PDE5 (aa 590-836 of WT, R732Y) | 524 | 525 |
| PDE5DD-134 | PDE5 (aa 590-836 of WT, R732H) | 526 | 527 |
| PDE5DD-135 | PDE5 (aa 590-836 of WT, R732S) | 528 | 529 |
| PDE5DD-136 | PDE5 (aa 590-836 of WT, R732T) | 530 | 531 |
| PDE5DD-137 | PDE5 (aa 590-836 of WT, R732D) | 532 | 533 |
| PDE5DD-138 | PDE5 (aa 590-836 of WT, R732E) | 534 | 535 |
| PDE5DD-139 | PDE5 (aa 590-836 of WT, R732Q) | 536 | 537 |
| PDE5DD-140 | PDE5 (aa 590-836 of WT, R732N) | 538 | 539 |
| PDE5DD-141 | PDE5 (aa 590-836 of WT, R732M) | 540 | 541 |
| PDE5DD-142 | PDE5 (aa 590-836 of WT, R732C) | 542 | 543 |
| PDE5DD-143 | PDE5 (aa 590-836 of WT, R732K) | 544 | 545 |
| PDE5DD-144 | PDE5 (aa 590-836 of WT, R732L) | 546 | 547 |
| PDE5DD-145 | PDE5 (aa 535-836 of WT, R732L) | 548 | 549 |
| PDE5DD-146 | PDE5 (aa 535-860 of WT, Y612F, | 550 | 551 |
| | R732L) | | |
| PDE5DD-147 | PDE5 (aa 535-860 of WT, Y612W, | 552 | 553 |
| | R732L) | | |
| PDE5DD-148 | PDE5 (aa 535-860 of WT, Y612A, | 554 | 555 |
| | R732L) | | |
| PDE5DD-149 | PDE5 (aa 535-860 of WT, F736A, | 556 | 557 |
| | D764N) | | |
| PDE5DD-150 | PDE5 (aa 535-860 of WT, R732L, | 558 | 559 |
| | D764N) | | |
| PDE5DD-151 | PDE5 (aa 535-860 of WT, R732L, | 560 | 561-562 |
| | F736A) | | |
| PDE5DD-152 | PDE5 (aa 535-860 of WT, H653A, | 563 | 564 |
| | R732L) | | |
| PDE5DD-153 | PDE5 (aa 535-860 of WT, R732L, | 565 | 566 |
| | D764A) | | |
| PDE5DD-154 | PDE5 (aa 535-860 of WT, L573M, | 567 | 568 |
| | F735L) | | |
| PDE5DD-155 | PDE5 (aa 535-860 of WT, Y709H, | 569 | 570 |
| | F787V) | | |
| PDE5DD-156 | PDE5 (aa 535-860 of WT, N605Y, | 571 | 572 |
| | I715K) | | |
| PDE5DD-157 | PDE5 (aa 535-860 of WT, I700F, | 573 | 574 |
| | E796G) | | |
| PDE5DD-158 | PDE5 (aa 535-860 of WT, D724G, | 575 | 576 |
| | K848N) | | |
| PDE5DD-159 | PDE5 (aa 535-860 of WT, I556S, | 577 | 578 |
| | E796D) | | |
| PDE5DD-160 | PDE5 (aa 535-860 of WT, L569M, | 579 | 580 |
| | T833S) | | |
| PDE5DD-161 | PDE5 (aa 535-860 of WT, V548M, | 581 | 582 |
| | D803N) | | |
| PDE5DD-162 | PDE5 (aa 535-860 of WT, G659A, | 583 | 584 |
| | T784I) | | |
| PDE5DD-163 | PDE5 (aa 535-860 of WT, H617L, | 585 | 586 |
| | A722V) | | |
| PDE5DD-164 | PDE5 (aa 535-860 of WT, N609Y, | 587 | 588 |
| | I799L) | | |
| PDE5DD-165 | PDE5 (aa 535-860 of WT, K770Q, | 589 | 590 |
| | K848N) | | |
| PDE5DD-166 | PDE5 (aa 535-860 of WT, V548M, | 591 | 592 |
| | F820I) | | |
| PDE5DD-167 | PDE5 (aa 535-860 of WT, S550F, | 593 | 594 |
| | L554R) | | |
| PDE5DD-168 | PDE5 (aa 535-860 of WT, D724G, | 595 | 596 |
| | D764G) | | |
| PDE5DD-169 | PDE5 (aa 535-860 of WT, Y728L, | 597 | 598 |
| | D764N) | | |
| PDE5DD-170 | PDE5 (aa 535-860 of WT, E682A, | 599 | 600 |
| | R732L) | | |
| PDE5DD-171 | PDE5 (aa 535-860 of WT, D724A, | 601 | 602 |
| | R732L) | | |
| PDE5DD-172 | PDE5 (aa 535-860 of WT, S562G, | 603 | 604 |
| | L727P, R847T) | | |
| PDE5DD-173 | PDE5 (aa 535-860 of WT, T571S, | 605 | 606 |
| | V585M, T723S) | | |
| PDE5DD-174 | PDE5 (aa 535-860 of WT, A722V, | 607 | 608 |
| | F755L, M760I) | | |
| PDE5DD-175 | PDE5 (aa 535-860 of WT, S542L, | 609 | 610 |
| | E708V, W772C) | | |
| PDE5DD-176 | PDE5 (aa 535-860 of WT, T712M, | 611 | 612 |
| | M758I, Q859R) | | |
| PDE5DD-177 | PDE5 (aa 535-860 of WT, H613L, | 613 | 614 |
| | D724Y, F755Y) | | |
| PDE5DD-178 | PDE5 (aa 535-860 of WT, L554R, | 615 | 616 |
| | Q589E, M691I) | | |
| PDE5DD-179 | PDE5 (aa 535-860 of WT, L554R, | 617 | 618 |
| | Q589E, A823T) | | |
| PDE5DD-180 | PDE5 (aa 535-860 of WT, F561L, | 619 | 620 |
| | G659A, T784I) | | |
| PDE5DD-181 | PDE5 (aa 535-860 of WT, W615R, | 621 | 622 |
| | T723R, A762T, E808G) | | |
| PDE5DD-182 | PDE5 (aa 535-860 of WT, L554R, | 623 | 624 |
| | Q586P, K710N, K730E) | | |
| PDE5DD-183 | PDE5 (aa 535-860 of WT, Q586L, | 625 | 626 |
| | S663Y, A762T, E808G) | | |
| PDE5DD-184 | PDE5 (aa 535-860 of WT, T571I, | 627 | 628 |
| | K604R, I706N, E795R) | | |
| PDE5DD-185 | PDE5 (aa 535-860 of WT, F564I, | 629 | 630 |
| | N662Y, H685L, L693P, F736I) | | |
| PDE5DD-186 | PDE5 (aa 535-860 of WT, P549S, | 631 | 632 |
| | F564I, R658H, A779T, R847G) | | |
| PDE5DD-187 | PDE5 (aa 535-860 of WT, R732D, | 6406 | 6407 |
| | F736S) | | |
| PDE5DD-188 | PDE5 (aa 535-860 of WT, R732E, | 6408 | 6409 |
| | F736D) | | |
| PDE5DD-189 | PDE5 (aa 535-860 of WT, R732V, | 6410 | 6411 |
| | F736G) | | |
| PDE5DD-190 | PDE5 (aa 535-860 of WT, R732W, | 6412 | 6413 |
| | F736G) | | |
| PDE5DD-191 | PDE5 (aa 535-860 of WT, R732W, | 6414 | 6415 |
| | F736V) | | |
| PDE5DD-192 | PDE5 (aa 535-860 of WT, R732L, | 6416 | 6417 |
| | F736W) | | |
| PDE5DD-193 | PDE5 (aa 535-860 of WT, R732P, | 6418 | 6419 |
| | F736Q) | | |
| PDE5DD-194 | PDE5 (aa 535-860 of WT, R732A, | 6420 | 6421 |
| | F736A) | | |
| PDE5DD-195 | PDE5 (aa 535-860 of WT, R732S, | 6422 | 6423 |
| | F736G) | | |
| PDE5DD-196 | PDE5 (aa 535-860 of WT, R732T, | 6424 | 6425 |
| | F736P) | | |
| PDE5DD-197 | PDE5 (aa 535-860 of WT, R732M, | 6426 | 6427 |
| | F736H) | | |
| PDE5DD-198 | PDE5 (aa 535-860 of WT, R732Y, | 6428 | 6429 |
| | F736M) | | |
| PDE5DD-199 | PDE5 (aa 535-860 of WT, R732P, | 6430 | 6431 |
| | F736D) | | |
| PDE5DD-200 | PDE5 (aa 535-860 of WT, R732P, | 6432 | 6433 |
| | F736G) | | |
| PDE5DD-201 | PDE5 (aa 535-860 of WT, R732W, | 6434 | 6435 |
| | F736L) | | |
| PDE5DD-202 | PDE5 (aa 535-860 of WT, R732L, | 6436 | 6437 |
| | F736S) | | |
| PDE5DD-203 | PDE5 (aa 535-860 of WT, R732D, | 6438 | 6439 |
| | F736T) | | |
| PDE5DD-204 | PDE5 (aa 535-860 of WT, R732L, | 6440 | 6441 |
| | F736V) | | |
| PDE5DD-205 | PDE5 (aa 535-860 of WT, R732G, | 6442 | 6443 |
| | F736V) | | |
| PDE5DD-206 | PDE5 (aa 535-860 of WT, R732W, | 6444 | 6445 |
| | F736A) | | |
| PDE5DD-207 | PDE5 (aa 535-860 of WT, C574N) | 6446 | 6447 |
| PDE5DD-208 | PDE5 (aa 535-860 of WT, E536K, | 6448 | 6449 |
| | I739W) | | |
| PDE5DD-209 | PDE5 (aa 535-860 of WT, H678F, | 6450 | 6451 |
| | S702F) | | |
| PDE5DD-210 | PDE5 (aa 535-860 of WT, E669G, | 6452 | 6453 |
| | I700T) | | |
| PDE5DD-211 | PDE5 (aa 535-860 of WT, G632S, | 6454 | 6455 |
| | I648T) | | |
| PDE5DD-212 | PDE5 (aa 535-774 of WT, L646S) | 6456 | 6457 |
| PDE5DD-213 | PDE5 (aa 535-860 of WT, A762V) | 6458 | 6459 |
| PDE5DD-214 | PDE5 (aa 535-860 of WT, D640N) | 6460 | 6461 |
| PDE5DD-215 | PDE5 (aa 535-860 of WT, N636S) | 6462 | 6463 |
| PDE5DD-216 | PDE5 (aa 535-860 of WT, Q623R, | 6464 | 6465 |
| | D654G, K741N) | | |
| PDE5DD-217 | PDE5 (aa 535-860 of WT, A673T, | 6466 | 6467 |
| | L756V, C846Y) | | |
| PDE5DD-218 | PDE5 (aa 535-860 of WT, V660A, | 6468 | 6469 |
| | L781F, R794G, C825R, E858G) | | |
| PDE5DD-219 | PDE5 (aa 535-860 of WT, E642G, | 6470 | 6471 |
| | G697D, I813T) | | |
| PDE5DD-220 | PDE5 (aa 535-860 of WT, M758T) | 6472 | 6473 |
| PDE5DD-221 | PDE5 (aa 535-860 of WT, K752E) | 6474 | 6475 |
| PDE5DD-222 | PDE5 (aa 535-860 of WT, C677Y, | 6476 | 6477 |
| | H685R, A722V) | | |
| PDE5DD-223 | PDE5 (aa 535-860 of WT, T639S, | 6478 | 6479 |
| | M816R) | | |
| PDE5DD-224 | PDE5 (aa 535-860 of WT, T537A, | 6480 | 6481 |
| | D558G, I706T, F744L, D764N) | | |
| PDE5DD-225 | PDE5 (aa 535-860 of WT, Q586R, | 6482 | 6483 |
| | D724G) | | |
| PDE5DD-226 | PDE5 (aa 535-860 of WT, F686S) | 6484 | 6485 |
| PDE5DD-227 | PDE5 (aa 535-860 of WT, E539G, | 6486 | 6487 |
| | L738I) | | |
| PDE5DD-228 | PDE5 (aa 535-860 of WT, Q635R, | 6488 | 6489 |
| | E753K, I813T) | | |
| PDE5DD-229 | PDE5 (aa 535-860 of WT, L672P, | 6490 | 6491 |
| | S836L) | | |
| PDE5DD-230 | PDE5 (aa 535-860 of WT, M691T, | 6492 | 6493 |
| | D764N) | | |
| PDE5DD-231 | PDE5 (aa 535-860 of WT, R807G) | 6494 | 6495 |
| PDE5DD-232 | PDE5 (aa 535-860 of WT, R577Q, | 6496 | 6497 |
| | C596R, V660A, I715V, E785K, | | |
| | L856P) | | |
| PDE5DD-233 | PDE5 (aa 535-860 of WT, I720V, | 6498 | 6499 |
| | F820S) | | |
| PDE5DD-234 | PDE5 (aa 535-860 of WT, S695G, | 6500 | 6501 |
| | E707K, I739M, C763R) | | |
| PDE5DD-235 | PDE5 (aa 535-860 of WT, Y709H, | 6502 | 6503 |
| | K812R, L832P) | | |
| PDE5DD-236 | PDE5 (aa 535-860 of WT, N583S, | 6504 | 6505 |
| | K752E, C846S) | | |
| PDE5DD-237 | PDE5 (aa 535-860 of WT, E682G, | 6506 | 6507 |
| | D748N) | | |
| PDE5DD-238 | PDE5 (aa 535-860 of WT, K591R, | 6508 | 6509 |
| | I643T, L856P) | | |
| PDE5DD-239 | PDE5 (aa 535-860 of WT, F619S, | 6510 | 6511 |
| | V818A, Y829C) | | |
| PDE5DD-240 | PDE5 (aa 535-860 of WT, V548E, | 6512 | 6513 |
| | Q589L, K633I, M681T, S702I, | | |
| | K752E, L781P, A857T) | | |
| PDE5DD-241 | PDE5 (aa 535-860 of WT, S652G, | 6514 | 6515 |
| | Q688R) | | |
| PDE5DD-242 | PDE5 (aa 535-860 of WT, E565G) | 6516 | 6517 |
| PDE5DD-243 | PDE5 (aa 535-860 of WT, I774V) | 6518 | 6519 |
| PDE5DD-244 | PDE5 (aa 535-860 of WT, K591R) | 6520 | 6521 |
| PDE5DD-245 | PDE5 (aa 535-860 of WT, F559S, | 6522 | 6523 |
| | Y709C, M760T) | | |
| PDE5DD-246 | PDE5 (aa 535-860 of WT, A649V, | 6524 | 6525 |
| | A650T, K730E, E830K) | | |
| PDE5DD-247 | PDE5 (aa 535-860 of WT, Y728C, | 6526 | 6527 |
| | Q817R) | | |
| PDE5DD-248 | PDE5 (aa 535-860 of WT, L595P, | 6528 | 6529 |
| | K741R) | | |
| PDE5DD-249 | PDE5 (aa 535-860 of WT, R577W, | 6530 | 6531 |
| | W615R, M805T, I821V) | | |

In one embodiment, the SRE may include at least one PDE5-derived drug responsive domain (DD) such as, but not limited to, PDE5DD-1, PDE5DD-2, PDE5DD-3, PDE5DD-4, PDE5DD-5, PDE5DD-6, PDE5DD-7, PDE5DD-8, PDE5DD-9, PDE5DD-10, PDE5DD-11, PDE5DD-12, PDE5DD-13, PDE5DD-14, PDE5DD-15, PDE5DD-16, PDE5DD-17, PDE5DD-18, PDE5DD-19, PDE5DD-20, PDE5DD-21, PDE5DD-22, PDE5DD-23, PDE5DD-24, PDE5DD-25, PDE5DD-26, PDE5DD-27, PDE5DD-28, PDE5DD-29, PDE5DD-30, PDE5DD-31, PDE5DD-32, PDE5DD-33, PDE5DD-34, PDE5DD-35, PDE5DD-36, PDE5DD-37, PDE5DD-38, PDE5DD-39, PDE5DD-40, PDE5DD-41, PDE5DD-42, PDE5DD-43, PDE5DD-44, PDE5DD-45, PDE5DD-46, PDE5DD-47, PDE5DD-48, PDE5DD-49, PDE5DD-50, PDE5DD-51, PDE5DD-52, PDE5DD-53, PDE5DD-54, PDE5DD-55, PDE5DD-56, PDE5DD-57, PDE5DD-58, PDE5DD-59, PDE5DD-60, PDE5DD-61, PDE5DD-62, PDE5DD-63, PDE5DD-64, PDE5DD-65, PDE5DD-66, PDE5DD-67, PDE5DD-68, PDE5DD-69, PDE5DD-70, PDE5DD-71, PDE5DD-72, PDE5DD-73, PDE5DD-74, PDE5DD-75, PDE5DD-76, PDE5DD-77, PDE5DD-78, PDE5DD-79, PDE5DD-80, PDE5DD-81, PDE5DD-82, PDE5DD-83, PDE5DD-84, PDE5DD-85, PDE5DD-86, PDE5DD-87, PDE5DD-88, PDE5DD-89, PDE5DD-90, PDE5DD-91, PDE5DD-92, PDE5DD-93, PDE5DD-94, PDE5DD-95, PDE5DD-96, PDE5DD-97, PDE5DD-98, PDE5DD-99, PDE5DD-100, PDE5DD-101, PDE5DD-102, PDE5DD-103, PDE5DD-104, PDE5DD-105, PDE5DD-106, PDE5DD-107, PDE5DD-108, PDE5DD-109, PDE5DD-110, PDE5DD-111, PDE5DD-112, PDE5DD-113, PDE5DD-114, PDE5DD-115, PDE5DD-116, PDE5DD-117, PDE5DD-118, PDE5DD-119, PDE5DD-120, PDE5DD-121, PDE5DD-122, PDE5DD-123, PDE5DD-124, PDE5DD-125, PDE5DD-126, PDE5DD-127, PDE5DD-128, PDE5DD-129, PDE5DD-130, PDE5DD-131, PDE5DD-132, PDE5DD-133, PDE5DD-134, PDE5DD-135, PDE5DD-136, PDE5DD-137, PDE5DD-138, PDE5DD-139, PDE5DD-140, PDE5DD-141, PDE5DD-142, PDE5DD-143, PDE5DD-144, PDE5DD-145, PDE5DD-146, PDE5DD-147, PDE5DD-148, PDE5DD-149, PDE5DD-150, PDE5DD-151, PDE5DD-152, PDE5DD-153, PDE5DD-154, PDE5DD-155, PDE5DD-156, PDE5DD-157, PDE5DD-158, PDE5DD-159, PDE5DD-160, PDE5DD-161, PDE5DD-162, PDE5DD-163, PDE5DD-164, PDE5DD-165, PDE5DD-166, PDE5DD-167, PDE5DD-168, PDE5DD-169, PDE5DD-170, PDE5DD-171, PDE5DD-172, PDE5DD-173, PDE5DD-174, PDE5DD-175, PDE5DD-176, PDE5DD-177, PDE5DD-178, PDE5DD-179, PDE5DD-180, PDE5DD-181, PDE5DD-182, PDE5DD-183, PDE5DD-184, PDE5DD-185, PDE5DD-186, PDE5DD-187, PDE5DD-188, PDE5DD-189, PDE5DD-190, PDE5DD-191, PDE5DD-192, PDE5DD-193, PDE5DD-194, PDE5DD-195, PDE5DD-196, PDE5DD-197, PDE5DD-198, PDE5DD-199, PDE5DD-200, PDE5DD-201, PDE5DD-202, PDE5DD-203, PDE5DD-204, PDE5DD-205, PDE5DD-206, PDE5DD-207, PDE5DD-208, PDE5DD-209, PDE5DD-210, PDE5DD-211, PDE5DD-212, PDE5DD-213, PDE5DD-214, PDE5DD-215, PDE5DD-216, PDE5DD-217, PDE5DD-218, PDE5DD-219, PDE5DD-220, PDE5DD-221, PDE5DD-222, PDE5DD-223, PDE5DD-224, PDE5DD-225, PDE5DD-226, PDE5DD-227, PDE5DD-228, PDE5DD-229, PDE5DD-230, PDE5DD-231, PDE5DD-232, PDE5DD-233, PDE5DD-234, PDE5DD-235, PDE5DD-236, PDE5DD-237, PDE5DD-238, PDE5DD-239, PDE5DD-240, PDE5DD-241, PDE5DD-242, PDE5DD-243, PDE5DD-244, PDE5DD-245, PDE5DD-246, PDE5DD-247, PDE5DD-248, and PDE5DD-249.

8. PPAR Gamma (PPARg) Derived Drug Responsive Domains (DDs)

In one embodiment, the SRE may include at least one drug responsive domain (DD) derived from a PPAR gamma (PPARg) protein or a fragment or variant thereof.

In one embodiment, the SRE comprises a region of the PPARg protein. The region of the PPARg protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

9. Estrogen Receptor (ER) Derived Drug Responsive Domains (DRDs)

In one embodiment, the SRE may include at least one drug responsive domain (DRD) derived from an Estrogen Receptor (ER) protein or a fragment or variant thereof.

In one embodiment, the SRE comprises a region of the ER protein. The region of the ER protein may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, or more than 450 amino acids in length. The region of the parent protein may be 5-50, 25-75, 50-100, 75-125, 100-150, 125-175, 150-200, 175-225, 200-250, 225-275, 250-300, 275-325, 300-350, 325-375, 350-400, 375-425, or 400-450 amino acids in length.

In one embodiment, the DRD may be derived from an ER protein and include at least one mutation. Non-limiting examples of mutations include K303R, N304S, T371A, L384M, M421G, N519S, G521G, Y537S.

In one embodiment, the DRD may be derived from an ER protein and include more than one mutation. Any of the mutations listed herein may be included in the DRD. Non-limiting examples of four mutations include L384M, M421G, G521R, Y537S. Non-limiting examples of six mutations include T371A, L384M, M421G, N519S, G521R, Y537S. Non-limiting examples of eight mutations include K303R, N304S, T371A, L384M, M421G, N519S, G521R, Y537S.

In some embodiments, DRDs derived from ER may comprise amino acids 2-595 of the parent ER sequence. This is referred to herein as an M1del mutation.

In one embodiment, the stimulus is a small molecule that binds to a SRE to post-translationally regulate protein levels.

In some embodiments, DRDs derived from ER may comprise amino acids 2-875 of the parent ER sequence. This is referred to herein as an M1del mutation.

In some embodiments, DDs are derived from a region of the ER protein. As a non-limiting example, the region is amino acid 305-549 of ER (SEQ ID NO: 76).

A non-limiting listing of ER drug responsive domain (amino acid and nucleic acid sequences) are listed in Table 6. The position of the mutated amino acid listed in Table 6 is relative to ER (Uniprot ID: P03372.2) of SEQ ID NO: 42. In Table 6, “del” means that the mutation is the deletion of the amino acid at that position relative to the wild type sequence.

TABLE 6
ER Derived Drug responsive domains (DDs)

| | | Amino | Nucleic |
|---|---|---|---|
| DRD | | acid | Acid |
| Identifier | ER Regions and Mutations | SEQ ID | SEQ ID |
| ERDD-1 | ER (aa 305-549 of WT, T371A, L384M, M421G, N519S, | 633 | 634-636 |
| | G521R, Y537S) | | |
| ERDD-2 | ER (aa 305-549 of WT, L384M, M421G, G521R, Y537S) | 637 | 638-640 |
| ERDD-3 | ER (aa 303-549 of WT, K303R, N304S, T371A, L384M, | 641 | — |
| | M421G, N519S, G521R, Y537S) | | |
| ERDD-6 | ER (aa 305-549 of WT, R335G, L384M, M421G, N519S, | 642 | 643 |
| | G521R, Y537S) | | |
| ERDD-7 | ER (aa 305-549 of WT, R335G, L384M, M421G, G521R, | 644 | 645 |
| | E523G, Y537S, A546T) | | |
| ERDD-8 | ER (aa 305-549 of WT, L384M; M421G; T431I; G521R, | 646 | 647 |
| | Y537S) | | |
| ERDD-9 | ER (aa 305-549 of WT, L384M, N413D, M421G, G521R, | 648 | 649 |
| | Y537S) | | |
| ERDD-10 | ER (aa 305-549 of WT, L384M, M421G, N519S, G521R, | 650 | 651 |
| | Y537S) | | |
| ERDD-11 | ER (aa 305-549 of WT, L384M, M421G, Q502R, G521R, | 652 | 653 |
| | Y537S) | | |
| ERDD-12 | ER (aa 305-549 of WT, S305N, L384M, M421G, G442V, | 654 | 655 |
| | G521R, Y537S) | | |
| ERDD-13 | ER (aa 305-549 of WT, L384M, N413F, M421G, G521R, | 656 | 657 |
| | Y537S) | | |
| ERDD-14 | ER (aa 305-549 of WT, L384M, N413L, M421G, G521R, | 658 | 659 |
| | Y537S) | | |
| ERDD-15 | ER (aa 305-549 of WT, L384M, N413Y, M421G, G521R, | 660 | 661 |
| | Y537S) | | |
| ERDD-16 | ER (aa 305-549 of WT, L384M, N413H, M421G, G521R, | 662 | 663 |
| | Y537S) | | |
| ERDD-17 | ER (aa 305-549 of WT, L384M, N413Q, M421G, G521R, | 664 | 665 |
| | Y537S) | | |
| ERDD-18 | ER (aa 305-549 of WT, L384M, N413I, M421G, G521R, | 666 | 667 |
| | Y537S) | | |
| ERDD-19 | ER (aa 305-549 of WT, L384M, N413M, M421G, | 668 | 669 |
| | G521R, Y537S) | | |
| ERDD-20 | ER (aa 305-549 of WT, L384M, N413K, M421G, G521R, | 670 | 671 |
| | Y537S) | | |
| ERDD-21 | ER (aa 305-549 of WT, L384M, N413V, M421G, G521R, | 672 | 673 |
| | Y537S) | | |
| ERDD-22 | ER (aa 305-549 of WT, L384M, N413S, M421G, G521R, | 674 | 675 |
| | Y537S) | | |
| ERDD-23 | ER (aa 305-549 of WT, L384M, N413C, M421G, G521R, | 676 | 677 |
| | Y537S) | | |
| ERDD-24 | ER (aa 305-549 of WT, L384M, N413W, M421G, | 678 | 679 |
| | G521R, Y537S) | | |
| ERDD-25 | ER (aa 305-549 of WT, L384M, N413P, M421G, G521R, | 680 | 681 |
| | Y537S) | | |
| ERDD-26 | ER (aa 305-549 of WT, L384M, N413R, M421G, G521R, | 682 | 683 |
| | Y537S) | | |
| ERDD-27 | ER (aa 305-549 of WT, L384M, N413T, M421G, G521R, | 684 | 685 |
| | Y537S) | | |
| ERDD-28 | ER (aa 305-549 of WT, L384M, N413A, M421G, G521R, | 686 | 687 |
| | Y537S) | | |
| ERDD-29 | ER (aa 305-549 of WT, L384M, N413E, M421G, G521R, | 688 | 689 |
| | Y537S) | | |
| ERDD-30 | ER (aa 305-549 of WT, L384M, N413G, M421G, G521R, | 690 | 691 |
| | Y537S) | | |
| ERDD-31 | ER (aa 305-549 of WT, L384M, M421G, Q502F, G521R, | 692 | 693 |
| | Y537S) | | |
| ERDD-32 | ER (aa 305-549 of WT, L384M, M421G, Q502L, G521R, | 694 | 695 |
| | Y537S) | | |
| ERDD-33 | ER (aa 305-549 of WT, L384M, M421G, Q502Y, G521R, | 696 | 697 |
| | Y537S) | | |
| ERDD-34 | ER (aa 305-549 of WT, L384M, M421G, Q502H, G521R, | 698 | 699 |
| | Y537S) | | |
| ERDD-35 | ER (aa 305-549 of WT, L384M, M421G, Q502I, G521R, | 700 | 701 |
| | Y537S) | | |
| ERDD-36 | ER (aa 305-549 of WT, L384M, M421G, Q502M, G521R, | 702 | 703 |
| | Y537S) | | |
| ERDD-37 | ER (aa 305-549 of WT, L384M, M421G, Q502N, G521R, | 704 | 705 |
| | Y537S) | | |
| ERDD-38 | ER (aa 305-549 of WT, L384M, M421G, Q502K, G521R, | 706 | 707 |
| | Y537S) | | |
| ERDD-39 | ER (aa 305-549 of WT, L384M, M421G, Q502V, G521R, | 708 | 709 |
| | Y537S) | | |
| ERDD-40 | ER (aa 305-549 of WT, L384M, M421G, Q502S, G521R, | 710 | 711 |
| | Y537S) | | |
| ERDD-41 | ER (aa 305-549 of WT, L384M, M421G, Q502C, G521R, | 712 | 713 |
| | Y537S) | | |
| ERDD-42 | ER (aa 305-549 of WT, L384M, M421G, Q502W, G521R, | 714 | 715 |
| | Y537S) | | |
| ERDD-43 | ER (aa 305-549 of WT, L384M, M421G, Q502P, G521R, | 716 | 717 |
| | Y537S) | | |
| ERDD-44 | ER (aa 305-549 of WT, L384M, M421G, Q502T, G521R, | 718 | 719 |
| | Y537S) | | |
| ERDD-45 | ER (aa 305-549 of WT, L384M, M421G, Q502A, G521R, | 720 | 721 |
| | Y537S) | | |
| ERDD-46 | ER (aa 305-549 of WT, L384M, M421G, Q502D, G521R, | 722 | 723 |
| | Y537S) | | |
| ERDD-47 | ER (aa 305-549 of WT, L384M, M421G, Q502E, G521R, | 724 | 725 |
| | Y537S) | | |
| ERDD-48 | ER (aa 305-549 of WT, L384M, M421G, Q502G, G521R, | 726 | 727 |
| | Y537S) | | |

In one embodiment, the SRE may include at least one ER-derived drug responsive domain (DRD) such as, but not limited to, ERDD-1, ERDD-2, ERDD-3, ERDD-6, ERDD-7, ERDD-8, ERDD-9, ERDD-10, ERDD-11, ERDD-12, ERDD-13, ERDD-14, ERDD-15, ERDD-16, ERDD-17, ERDD-18, ERDD-19, ERDD-20, ERDD-21, ERDD-22, ERDD-23, ERDD-24, ERDD-25, ERDD-26, ERDD-27, ERDD-28, ERDD-29, ERDD-30, ERDD-31, ERDD-32, ERDD-33, ERDD-34, ERDD-35, ERDD-36, ERDD-37, ERDD-38, ERDD-39, ERDD-40, ERDD-41, ERDD-42, ERDD-43, ERDD-44, ERDD-45, ERDD-46, ERDD-47, and ERDD-48.

In some embodiments, ERDD may include one or more mutations selected from but not limited to N413T, N413H, N413A, N413Q, N413V, N413C, N413K, N413M, N413R, N413S, N413W, N413I, N413E, N413L, N413P, N413F, N413Y, N413G Q502D, Q502H, Q502E, Q502V, Q502A, Q502T, Q502N, Q502K, Q502S, Q502L, Q502Y, Q502W, Q502F, Q502I, Q502G, Q502P, Q502M, Q502C, L384M, M421G, G521R, Y537S, K303R, N304S, S305N, R335G, T371A, T431I, N519S, E523G, A546T, and G442V.

The present disclosure provides compositions that include effector modules with SREs derived from the whole or portion of a parent protein, such as PDE5 and a first payload which includes in whole or in part the human CD40L (SEQ ID NO: 3820), or a mutant thereof. In one embodiment, the SRE includes amino acids 535-860 of PDE5. In some embodiments, the SRE may include one or more mutations compared to the parent protein. The SRE may include but is not limited to SEQ ID NO: 294, 296, 298, 300, 302, 306, 308, 313, 315, 317, 318, 319, 320, 321, 322, 323, 324, 325, 326, 327, 328, 329, 330, 331, 332, 333, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 375, 378, 381, 384, 387, 389, 391, 394, 397, 400, 403, 406, 409, 412, 415, 417, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 6406, 6408, 6410, 6412, 6414, 6416, 6418, 6420, 6422, 6424, 6426, 6428, 6430, 6432, 6434, 6436, 6438, 6440, 6442, 6444, 6446, 6448, 6450, 6452, 6454, 6456, 6458, 6460, 6462, 6464, 6466, 6468, 6470, 6472, 6474, 6476, 6478, 6480, 6482, 6484, 6486, 6488, 6490, 6492, 6494, 6496, 6498, 6500, 6502, 6504, 6506, 6508, 6510, 6512, 6514, 6516, 6518, 6520, 6522, 6524, 6526, 6528, or 6530.

In one embodiment, the SRE includes the mutation R732L and SRE may include the amino acid sequence of SEQ ID NO: 308.

The SRE described herein may be responsive to or interact with at least one stimulus. In one aspect, the stimulus may be a small molecule such as but not limited to Vardenafil, Tadalafil or Sildenafil. In one aspect, the small molecule is Vardenafil.

In some aspects, the effector module may include a linker. Such a linker may operably link the SRE to the first payload. In some embodiments, the linker may be a Glycine and Serine containing linker, or a linker known in the art, for example, in any one or more of the following publications, WO 2018/161000; WO 2018/231759; WO 2019/241315; WO 2018/160993; WO 2018/237323; and WO 2018/161038. In one embodiment, the linker comprises the amino acid sequence of SEQ ID NO: 6532.

In one embodiment, the effector module may be SEQ ID NO: 6534.

Also provided herein are polynucleotides encoding the compositions described herein as well as vectors encoding the polynucleotides. The present disclosure also provides pharmaceutical compositions that include the compositions described herein and a pharmaceutically acceptable excipient as well as immune cells expressing the compositions described herein.

Also provided herein is a method of reducing tumor burden in a subject. The methods may include the steps of administering to the subject a therapeutically effective amount of immune cells. The immune cells may include or express a composition comprising a stimulus response element (SRE) operably linked to a first payload. The first payload may include in whole or in part the human CD40L (SEQ ID NO: 3820) or a mutant thereof. The immune cell may also express a pharmaceutical composition that includes the compositions described herein. The methods also involve administering to the subject, a therapeutically effective amount of a stimulus. In one embodiment, the stimulus is a ligand. The ligand may modulate the expression of the first payload to reduce the tumor burden. In some embodiments, the ligand is Vardenafil, Tadalafil, Sildenafil, Shield-1, or Trimethoprim.

The present disclosure also provides methods for activating dendritic cells in a subject. The methods may include the steps of administering to the subject a therapeutically effective amount of immune cells. The immune cells may include or express a composition comprising a stimulus response element (SRE) operably linked to a first payload. In one embodiment, the immune cell is a T cell. The first payload may include, in whole or in part, human CD40L (SEQ ID NO: 3820) or a mutant thereof. The immune cell may also express a pharmaceutical composition that includes the compositions described herein. The methods also involve administering to the subject a therapeutically effective amount of a stimulus. In one embodiment, the stimulus is a ligand. The methods further may include measuring the dendritic cell activation marker IL12 in the subject in response to the ligand to determine dendritic cell activation. In one embodiment, the dendritic cell may be a myeloid dendritic cell, a plasmacytoid dendritic cell, a CD14+ dendritic cell, a Langerhans cell, or a microglia. In one aspect, the dendritic cell is a myeloid dendritic cell.

Compositions described herein may further include a CAR. The CAR may include (a) an extracellular target moiety; (b) a transmembrane domain; (c) an intracellular signaling domain; and (d) optionally, one or more co-stimulatory domains. The extracellular target moiety of the CAR may be an scFv. In one embodiment, the scFv may be a CD19 scFv. In some embodiments, the co-stimulatory domain may be present.

10. Stimuli of Tunable Protein Expression Systems

A tunable protein expression system of the present disclosure can be responsive to a stimulus.

In some embodiments, a stimulus is a ligand. Ligands may be nucleic acid-based, protein-based, lipid based, organic, inorganic or any combination of the foregoing. In some embodiments, ligands may be synthetic molecules. In some embodiments, ligands may be small molecule therapeutic compounds. In some embodiments, ligands may be small molecule drugs previously approved by a regulatory agency, such as the FDA.

As described in the present disclosure, a tunable protein expression system can exhibit ligand-dependent activity. A ligand can bind to a DRD and stabilize an appended or operably linked protein of interest. Ligands that are known to bind candidate DRDs can be tested for their effect on the activity of a tunable protein expression system.

In some embodiments, a ligand is cell permeable. In some embodiments, a ligand may be designed to be lipophilic to improve cell permeability.

In some embodiments, a ligand is a small molecule. A small molecule ligand may be clinically approved to be safe and have appropriate pharmaceutical kinetics and distribution.

In some embodiments, the ligand may be complexed or bound to one or more other molecules such as, but not limited to, another ligand, a protein, peptide, nucleic acid, lipid, lipid derivative, sterol, steroid, metabolite, metabolite derivative or small molecule. In some embodiments, the ligand stimulus is complexed or bound to one or more different kinds and/or numbers of other molecules. In some embodiments, the ligand stimulus is a multimer of the same kind of ligand. In some embodiments, the ligand stimulus multimer comprises 2, 3, 4, 5, 6, or more monomers.

11. DHFR Ligands

In some embodiments, a ligand of the present disclosure binds to dihydrofolate reductase. In some embodiments, the ligand binds to and inhibits dihydrofolate reductase function and is herein referred to as a dihydrofolate inhibitor.

In some embodiments, the ligand may be a selective inhibitor of human DHFR. Ligands of the disclosure may also be selective inhibitors of dihydrofolate reductases of bacteria and parasitic organisms such as Pneumocystis spp., Toxoplasma spp., Trypanosoma spp., Mycobacterium spp., and Streptococcus spp. Ligands specific to other DHFR may be modified to improve binding to human dihydrofolate reductase.

Examples of dihydrofolate inhibitors include, but are not limited to, Trimethoprim (TMP), Methotrexate (MTX), Pralatrexate, Piritrexim, Pyrimethamine, Talotrexin, Chloroguanide, Pentamidine, Trimetrexate, aminopterin, Cl 898 trihydrochloride, Pemetrexed Disodium, Raltitrexed, Sulfaguanidine, Folotyn, Iclaprim and Diaveridine.

In some embodiments, ligands of the present disclosure may include dihydrofolic acid or any of its derivatives that may bind to human DHFR. In some embodiments, the ligands of the present disclosure may be 2,4, diaminohetrocyclic compounds. In some embodiments, the 4-oxo group in dihydrofolate may be modified to generate DHFR inhibitors. In one example, the 4-oxo group may be replaced by 4-amino group. Various diamino heterocycles, including pteridines, quinazolines, pyridopyrimidines, pyrimidines, and triazines, may also be used as scaffolds to develop DHFR inhibitors and may be used according to the present disclosure.

In some embodiments, ligands include TMP-derived ligands containing portions of the ligand known to mediate binding to DHFR. Ligands may also be modified to reduce off-target binding to other folate metabolism enzymes and increase specific binding to DHFR.

12. ER Ligands

In some embodiments, a ligand of the present disclosure binds to ER. Ligands may be agonists or antagonists. In some embodiments, the ligand binds to and inhibits ER function and is herein referred to as an ER inhibitor. In some embodiments, the ligand may be a selective inhibitor of human ER. Ligands of the disclosure may also be selective inhibitors of ER of other species. Ligands specific to other ER may be modified to improve binding to human ER.

Ligands may be ER agonists such as but not limited to endogenous estrogen 17b-estradiol (E2) and the synthetic nonsteroidal estrogen diethylstilbestrol (DES). In some embodiments. The ligands may be ER antagonists, such as ICI-164,384, RU486, tamoxifen, 4-hydroxytamoxifen (4-OHT), fulvestrant, oremifene, lasofoxifene, clomifene, femarelle and ormeloxifene and raloxifene (RAL).

In some embodiments, the stimulus of the current disclosure may be ER antagonists such as, but not limited to, Bazedoxifene and/or Raloxifene.

In some embodiments, ligands include Bazedoxifene-derived ligands containing portions of the ligand known to mediate binding to ER. Ligands may also be modified to reduce off-target binding to other folate metabolism enzymes and increase specific binding to ER derived DRDs.

13. Phosphodiesterase Ligands

In some embodiments, ligands of the present disclosure bind to phosphodiesterases. In some embodiments, the ligands bind to and inhibit phosphodiesterase function and are herein referred to as phosphodiesterase inhibitors.

In some embodiments, the ligand is a small molecule that binds to phosphodiesterase 5. In one embodiment, the small molecule is an hPDE5 inhibitor. Examples of hPDE5 inhibitors include, but are not limited to, Sildenafil, Vardenafil, Tadalafil, Avanafil, Lodenafil, Mirodenafil, Udenafil, Benzamidenafil, Dasantafil, Beminafil, SLx-2101, LAS 34179, UK-343,664, UK-357903, UK-371800, and BMS-341400.

In some embodiments, ligands include sildenafil-derived ligands containing portions of the ligand known to mediate binding to hPDE5. Ligands may also be modified to reduce off-target binding to phosphodiesterases and increase specific binding to hPDE5.

In some embodiments, the stimulus may be a ligand that binds to more than one phosphodiesterase. In one embodiment, the stimulus is a pan-phosphodiesterase inhibitor that may bind to two or more hPDEs such as Aminophyline, Paraxanthine, Pentoxifylline, Theobromine, Dipyridamole, Theophyline, Zaprinast, Icariin, CDP-840, Etazolate and Glaucine.

14. FKBP Ligands

In some embodiments, ligands of the present disclosure bind to FKBP, including human FKBP. In some embodiments, the ligand is SLF or Shield-1.

15. Stabilization and Destabilization Ratio

In some embodiments, the present disclosure provides methods for modulating protein, expression, function or level by measuring the stabilization ratio and destabilization ratio. As used herein, the stabilization ratio may be defined as the ratio of expression, function or level of a protein of interest in response to the stimulus to the expression, function or level of the protein of interest in the absence of the stimulus specific to the SRE. In some aspects, the stabilization ratio is at least 1, such as by at least 1-10, 1-20, 1-30, 1-40, 1-50, 1-60, 1-70, 1-80, 1-90, 1-100, 20-30, 20-40, 20-50, 20-60, 20-70, 20-80, 20-90, 20-95, 20-100, 30-40, 30-50, 30-60, 30-70, 30-80, 30-90, 30-95, 30-100, 40-50, 40-60, 40-70, 40-80, 40-90, 40-95, 40-100, 50-60, 50-70, 50-80, 50-90, 50-95, 50-100, 60-70, 60-80, 60-90, 60-95, 60-100, 70-80, 70-90, 70-95, 70-100, 80-90, 80-95, 80-100, 90-95, 90-100 or 95-100. As used herein, the destabilization ratio may be defined as the ratio of expression, function or level of a protein of interest in the absence of the stimulus specific to the effector module to the expression, function or level of the protein of interest, that is expressed constitutively and in the absence of the stimulus specific to the SRE. As used herein “constitutively” refers to the expression, function or level of a protein of interest that is not linked to an SRE, and is therefore expressed both in the presence and absence of the stimulus. In some aspects, the destabilization ratio is at least 0, such as by at least 0.1, 0.2, 0.3, 0.4, 0.5, 0.6, 0.7, 0.8, 0.9, or at least, 0-0.1, 0-0.2, 0-0.3, 0-0.4, 0-0.5, 0-0.6, 0-0.7, 0-0.8, 0-0.9, 0.1-0.2, 0.1-0.3, 0.1-0.4, 0.1-0.5, 0.1-0.6, 0.1-0.7, 0.1-0.8, 0.1-0.9, 0.2-0.3, 0.2-0.4, 0.2-0.5, 0.2-0.6, 0.2-0.7, 0.2-0.8, 0.2-0.9, 0.3-0.4, 0.3-0.5, 0.3-0.6, 0.3-0.7, 0.3-0.8, 0.3-0.9, 0.4-0.5, 0.4-0.6, 0.4-0.7, 0.4-0.8, 0.4-0.9, 0.5-0.6, 0.5-0.7, 0.5-0.8, 0.5-0.9, 0.6-0.7, 0.6-0.8, 0.6-0.9, 0.7-0.8, 0.7-0.9 or 0.8-0.9.

In some embodiments, the SRE of the effector module may stabilize the payload of interest by a stabilization ratio of 1 or more, wherein the stabilization ratio may comprise the ratio of expression, function or level of the payload of interest in the presence of the stimulus to the expression, function or level of the payload of interest in the absence of the stimulus.

In some embodiments, the SRE may destabilize the immunotherapeutic agent by a destabilization ratio between 0, and 0.09, wherein the destabilization ratio may comprise the ratio of expression, function or level of the payload of interest in the absence of the stimulus specific to the SRE to the expression, function or level of the payload of interest that is expressed constitutively, and in the absence of the stimulus specific to the SRE.

16. Additional Effector Module Features

The effector module of the present disclosure may further comprise additional components that may be operably linked to either the DRD or the payload or both. In some embodiments, the additional components may include a signal sequence which regulates the distribution of the payload of interest, a cleavage and/or processing feature which facilitate cleavage of the payload from the effector module construct, a targeting and/or penetrating signal which can regulate the cellular localization of the effector module, a tag, and/or one or more linker sequences which link different components of the effector module, regulatory elements, polyadenylation sequences, transmembrane domains, intra tail domains, hinges, tags, cleavage site, leader sequences. Examples of such additional effector module components are described in WO 2018/161000; WO 2018/231759; WO 2019/241315; WO 2018/160993; WO 2018/237323; and WO 2018/161038. In one embodiment, the transmembrane domain region of a first payload may be replaced with a transmembrane domain, variant or fragment thereof, from a second parent protein.

17. Payloads

As used herein a “payload” or “protein of interest” (used interchangeably herein) is any polypeptide, protein or portion thereof that is linked, appended, or operably linked to a DRD of the present disclosure.

Payloads may include any polypeptide or any protein or fragment thereof. A payload may be a wild-type sequence, a fragment of a wild-type sequence and/or comprise one or more mutations. A payload may be a natural protein from an organism genome, or variants, mutants, and derivatives thereof. The natural protein may be from, for example, a mammalian organism, a bacterium, and a virus. A payload may be a protein or polypeptide encoded by a recombinant nucleic acid molecule, a fusion or chimeric polypeptide, or a polypeptide that functions as part of a protein complex.

In one example, a payload may be a polypeptide encoded by a nucleic acid sequence from a human genome.

In some embodiments, a payload may be a variant sequence of a parent polypeptide. In some aspects, the variant sequence may have the same or a similar activity as the reference sequence. Alternatively, the variant may have an altered activity (e.g., increased or decreased) relative to a reference sequence. Generally, variants of a particular polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polypeptide as determined by sequence alignment programs known to those skilled in the art.

18. Therapeutic Agents as Payloads

In some embodiments, payloads of the present disclosure may be therapeutic agents. For example, a payload may be a cancer therapeutic agent, a therapeutic agent for an autoimmune disease, an immunotherapeutic agent, an anti-inflammatory agent, an anti-pathogen agent or a gene therapy agent. In some aspects, the immunotherapeutic agent may be an antibody and fragments and variants thereof, a TCR receptor, a chimeric antigen receptor (CAR), a chimeric switch receptor, an antagonist of a co-inhibitory molecule, an agonist of a co-stimulatory molecule, a cytokine, a cytokine receptor, a chemokine, a chemokine receptor, a metabolic factor, a coagulation factor, an enzyme, a homing receptor and a safety switch.

In some embodiments, payloads of the present disclosure may be immunotherapeutic agents that induce immune responses in an organism. The immunotherapeutic agent may be, but is not limited to, an antibody and fragments and variants thereof, a chimeric antigen receptor (CAR), a chimeric switch receptor, a cytokine, chemokine, a cytokine receptor, a chemokine receptor, a cytokine-cytokine receptor fusion polypeptide, or any agent that induces an immune response, and may include any agent that alters the activity, function or response of an immune cell. In one embodiment, the immunotherapeutic agent induces an anti-cancer immune response in a cell, or in a subject.

19. Cytokines, Chemokines and Other Soluble Factors as Payloads

In some embodiments, payloads of the present disclosure may be cytokines, chemokines, growth factors, and soluble proteins produced by immune cells, cancer cells and other cell types, which act as chemical communicators between cells and tissues within the body. These proteins mediate a wide range of physiological functions, from effects on cell growth, differentiation, migration and survival, to a number of effector activities. For example, activated T cells produce a variety of cytokines for cytotoxic function to eliminate tumor cells.

In some embodiments, payloads of the present disclosure may be cytokines, and fragments, variants, analogs and derivatives thereof, including but not limited to interleukins, tumor necrosis factors (TNFs), interferons (IFNs), TGF beta and chemokines. In some embodiments, payloads of the present invention may be cytokines that stimulate immune responses. In other embodiments, payloads of the invention may be antagonists of cytokines that negatively impact anti-cancer immune responses.

For example, the transmembrane of the first payload may be replaced with any of the transmembrane domain, variants or fragments thereof.

In some embodiments, the payload may be a fusion protein comprising any of the immunotherapeutic agents described and ubiquitin. Within the fusion protein, the ubiquitin may be positioned at the N terminus and the immunotherapeutic agent may be positioned at the C terminus. In one aspect, the immunotherapeutic agent may itself be a fusion protein and the ubiquitin may be located in between the proteins that are fused. The payloads may include a single ubiquitin protein or a chain of ubiquitin proteins. The ubiquitin protein may be linked to the immunotherapeutic agent through a single amino acid.

20. Immunotherapeutic Agents

In some embodiments, payloads of the present disclosure may be immunotherapeutic agents that induce immune responses in an organism. The immunotherapeutic agent may be, but is not limited to, an antibody and fragments and variants thereof, a chimeric antigen receptor (CAR), a chimeric switch receptor, a cytokine, chemokine, a cytokine receptor, a chemokine receptor, a cytokine-cytokine receptor fusion polypeptide, or any agent that induces an immune response. In one embodiment, the immunotherapeutic agent induces an anti-cancer immune response in a cell, or in a subject

21. CD40L

In various embodiments, payloads of the present disclosure include an immunotherapeutic agent. In various embodiments, the immunotherapeutic agent is a CD40 ligand (CD40L) also known as CD154 or TNFRF5, or a mutant comprising one or more amino acid substitutions, deletions or additions to the wild-type sequence of human CD40L. CD40L belongs to the TNF super family and is primarily expressed on T cells. CD40L binds to CD40 expressed on a multitude of immune cells, and initiates a cascade of cellular responses depending on the cell type. CD40L may also bind to α5β1 integrin and αIIbβ3 integrins. CD40L is a type II membrane polypeptide having a cytoplasmic domain at its N-terminus, a transmembrane region and then an extracellular domain at its C-terminus. In some embodiments, the CD40L of the present disclosure may be engineered to bind to only one of its binding partners, e.g. CD40. In some aspects, the CD40L described herein may be capable of binding to all of its cognate binding partners.

Unless otherwise indicated the full length CD40L is designated herein as “CD40L.” The nucleotide and amino acid sequence of CD40L from mouse and human is well known in the art and can be found, for example, in U.S. Pat. No. 5,962,406 (Armitage et al.). Also included within the meaning of CD40 ligand are variations in the sequence including conservative amino acid changes and the like which do not alter the ability of the ligand to elicit an immune response to a mucin.

CD40L may bind to CD40 expressed in but not limited to Antigen Presenting Cells (APCs), B cells, monocytes, macrophages, platelets, neutrophils, dendritic cells, endothelial cells, and αSMC (smooth muscle cells). Binding of CD40L to CD40 expressed on dendritic cells may promote dendritic cell (DC) licensing. DCs may be converted to a functional state by an antigen-specific T helper cell in order to activate cytotoxic CD8+ T cells, a process referred to as DC licensing. CD40 engagement on DCs results in DC stimulation as evidenced by the surface expression of costimulatory and MHC molecules; proinflammatory cytokine production (e.g. IL12 and TNF) as well as epitope spreading.

In some embodiments, CD40L regulated by the tunable protein expression systems described herein may be utilized for the therapy of solid, immunogenic tumors. CD40L may improve the efficacy of solid tumor targeted T cells in immunogenic tumors by activating adaptive and innate immune responses in situ. Regulatable CD40L based biocircuit systems described herein may be desirable since the expression of the endogenous CD40L in T cells is transient. Further, the tumor microenvironment is rich in sheddases that may cleave the endogenous CD40L expressed by T cells. Exogenously expressed constitutive CD40L expression may result in liver toxicity and excessive B cell proliferation resulting in lymphomas (Schmitz et al (2006) Hepatology 44(2):430-9, Vonderheide et al (2007) J Clin Oncol. 1; 25(7):876-83, Sacco et al (2000) Cancer Gene Ther.; 7(10):1299-306); the contents of each of which are incorporated by reference in their entirety). Constitutive (unregulated) expression may lead to CRS, thromboembolic syndromes, autoimmune reactions, AICD due to hyper-immune stimulation and tumor angiogenesis, thereby creating a need for the biocircuits of the disclosure.

In some embodiments, the immunotherapeutic agent may be a multimer of CD40L molecules such as but not limited to a dimer, a trimer, a tetramer, a pentamer, a hexamer, a septamer, or a heptamer. In one embodiment, the CD40L may form a trimer. Multimerization of CD40L may enhance the signaling via the CD40L/CD40 axis. Binding of trimeric CD40L to CD40 may also initiate CD40 clustering and TRAF activation ultimately leading to NF-κB activation.

CD40L described herein may be resistant to proteinases and sheddases such as those found in the tumor microenvironment e.g. ADAM10, or ADAM17. The heightened activity of ADAM17 in the tumor microenvironment has been associated with diminished signaling via the CD40/CD40L axis (see Lowe and Corvaia (2016), Int J Cancer Clin Res, 3:058; the contents of which are incorporated by reference in their entirety).

In some aspects, the CD40L may be co-expressed with a chimeric antigen receptor. CD40L expressed on CART cells may increase the function of CART cells and bystander effector cells via activation of CD40+ immune cells such as but not limited to dendritic cells, macrophages, myeloid cells, B cells, platelets, endothelial cells, epithelial cells, and fibroblasts in the tumor microenvironment as well as the tumor cells themselves. In one embodiment, the payload may be bicistronic construct comprising CD40L and CD19CAR with CD28 and CD3Zeta co-stimulatory domains (see Curran et al. (2015) Mol Ther. 23: 4; 769-778; the contents of which are incorporated by reference in their entirety).

In some embodiments, cells of the present disclosure may also be engineered to express chimeric antigens receptors described herein in conjunction with CD40L. CD40L may be expressed constitutively or may be use as a payload in the effector modules of the present disclosure. CD40L is involved in dendritic cell antigen presentation; production of IL12, and the generation of CD8+ T-cell immunity. Any of the methods described by Curren et al. to enhance antitumor efficacy of CARs using CD40L may be useful in the present disclosure (Curren et al. Mol Ther. 2015 April; 23(4): 769-778; the contents of which are incorporated by reference in their entirety). In one embodiment, agonistic CD40 antibodies may be useful in the present disclosure. CD40 monoclonal antibodies have shown clinical activity in the absence of disabling toxicity.

The combination of the T regulatory cells, myeloid derived suppressor cells (MDSCs) and the extensive stromal networks within the tumor microenvironment (TME) can dampen the antitumor immune response by preventing T-cell infiltration and/or activation by current immunotherapies (see Ma et al. A CD40 agonist and PD-1 antagonist antibody reprogram the microenvironment of non-immunogenic tumors to allow T cell-mediated anticancer activity. Cancer Immunol Res Jan. 14, 2019; doi: 10.1158/2326-606.CIR-18-0061; the contents of which are herein incorporated by reference in their entireties). Current CAR T therapies are not effective as the therapeutics have immunosuppression, tumor antigen escape, insufficient CAR T expansion and healthy tissue toxicity.

The present disclosure addresses these issues with the utilization of an effector module with CD40L as an immunotherapeutic agent, fused directly or indirectly to an SRE comprising a DRD described herein. The CD40L may not be the only immunotherapeutic agent in the effector module. The effector module may also include a CAR construct. The combination of the CD40L and CAR as the immunotherapeutic agent and an SRE may cause any of the following alone or in combination, (1) repolarization of the CD40+ macrophages in the tumor microenvironment to a proinflammatory state, (2) activation of CD40+ dendritic cells to promote epitope spreading which can decrease tumor antigen escape (e.g., decrease the loss of CAR targeted antigens), (3) reverse signaling and cytokine production to enhance the antigen-dependent T cell expansion, and (4) regulatable protein production from the SRE which lowers toxicity of the therapeutic to healthy tissue. As a non-limiting example, an effector module including an SRE fused to a CD40L and CAR immunotherapeutic agent may be used to overcome the loss of CAR targeted antigens (e.g., antigen escape) by causing dendritic cells to recruit tumor infiltration lymphocytes (TILs) which results in the expansion of the group of anti-tumor specific T cells. As a non-limiting example, an effector module including an SRE fused to a CD40L and CAR immunotherapeutic agent may be used to reduce the constraint of the tumor microenvironment (TME) of solid tumors by repolarizing tumor associated macrophages (TAMs) from a suppressive to an inflammatory phenotype. As a non-limiting example, an effector module including an SRE fused to a CD40L and CAR immunotherapeutic agent may be used to increase CAR T cell expansion by causing antigen-dependent T cell expansion.

In some embodiments, the effector module comprises at least one immunotherapeutic agent. In one embodiment, the immunotherapeutic agent is CD40L. In one embodiment, the effector module comprises two or more immunotherapeutic agents which may be the same type such as two antibodies, or different types such as a CD40L and a CAR construct.

Provided herein are compositions for inducing an immune response in a cell or a subject. The compositions may include an effector module. The effector module may include a stimulus response element (SRE) operably linked to a first payload. The first payload may include in whole or in part the human CD40L (SEQ ID NO: 3820). In one embodiment, the first payload is the whole CD40L (SEQ ID NO: 3820).

In one aspect, the first payload may be a region of CD40L (SEQ ID NO: 3820). In one aspect, the region of CD40L may include amino acids 113 to 269 of SEQ ID NO: 3820 (SEQ ID NO: 3822). In one embodiment, the region of CD40L may include amino acids 12-261 of SEQ ID NO: 3820 (SEQ ID NO: 3824). In one embodiment, the region of CD40L may include amino acids 14-261 of SEQ ID NO: 3820 with a deletion in amino acids 110-116 of SEQ ID NO: 3820 (SEQ ID NO: 3826).

The SRE of the effector module may be derived from the whole or a portion of at least one parent protein, said parent protein selected from the group consisting of ER, ecDHFR, FKBP, PDE5, and hDHFR.

In one embodiment, the SRE may include one or more mutations as compared to the parent protein.

In one embodiment, the SRE may be derived from ER and the SRE may include but is not limited to the amino acid sequences listed in Table 6.

In one embodiment, the SRE may be derived from ecDHFR and the SRE may include but is not limited to the amino acid sequences listed in Table 3.

In one embodiment, the SRE may be derived from FKBP and the SRE may include but is not limited to the amino acid sequences listed in Table 4.

In one embodiment, the SRE may be derived from PDE5 and the SRE may include but is not limited to the amino acid sequences listed in Table 5.

In some aspects, the SRE may be responsive to or interacts with at least one stimulus. In one aspect, the effector module may include a second payload. In one embodiment, the second payload may be an immunotherapeutic agent. In some embodiments, the immunotherapeutic agent may be a Chimeric Antigen Receptor (CAR). The CAR described herein may include (a) an extracellular target moiety; (b) a transmembrane domain; (c) an intracellular signaling domain; and (d) optionally, one or more co-stimulatory domains.

The extracellular target moiety of the CAR may be an scFv. In some aspects, the extracellular target moiety may be an scFv. In one embodiment, the scFv may be a CD19 scFv. In some embodiments, the CAR includes a transmembrane domain. In some embodiments, the CAR includes an intracellular domain. In some embodiments, the CAR includes a co-stimulatory domain.

Also provided herein is a polynucleotide encoding the compositions of described herein a vector expressing the polynucleotide, as well as a pharmaceutical composition which include the compositions described herein and a pharmaceutically acceptable excipient.

The present disclosure also provides an immune cell for various methods of treatment disclosed herein, for example, for the treatment of cancer and adoptive cell transfer which expresses the pharmaceutical compositions. The immune cell may be, for example, a T cell (e.g CD8+ T cell, a CD4+ T cell), a natural killer (NK) cell, a NKT cell, a cytotoxic T lymphocyte (CTL), a tumor infiltrating lymphocyte (TIL), a memory T cell, a regulatory T (Treg) cell, a cytokine-induced killer (CIK) cell, a dendritic cell, a human embryonic stem cell, a mesenchymal stem cell, a hematopoietic stem cell, or a mixture thereof. In one embodiment, the immune cell is a dendritic cell. In one embodiment the immune cell is a CD8+ T cell. In one aspect, the immune cell is a CD4+ T cell.

Also provided herein are methods of inducing an immune response in a subject. Such methods may include administering to the subject, an effective amount of the immune cell described herein. The immune cell, wherein the immune cell expresses an effector module comprising a stimulus response element (SRE) operably linked to a first payload. The first payload may include in whole or in part, the human CD40L. The SREs expressed by the immune cell may be responsive to or interact with at least one stimulus. The method may further involve exposing the subject to the stimulus, causing the CD40L expression to be modulated. The modulation of CD40L expression induces the immune response. In some aspects, the method may further comprise administering to the subject, an effective amount of CD40 positive cells. In some aspects, the CD40 positive cell may be a dendritic cell, a macrophage, a myeloid cell, a B cell, a platelet, an endothelial cell, an epithelial cell, and a fibroblast.

The compositions described herein may be used for inducing an immune response. Such compositions may include (a) a first immune cell capable of expressing an effector module that includes CD40L (SEQ ID NO: 3820), or a mutant CD40L as its first payload; as well as a second immune cell expressing CD40L. The first immune cell and the second immune cell may be independently selected from a T cell (e.g CD8+ T cell, a CD4+ T cell), a natural killer (NK) cell, a NKT cell, a cytotoxic T lymphocyte (CTL), a tumor infiltrating lymphocyte (TIL), a memory T cell, a regulatory T (Treg) cell, a cytokine-induced killer (CIK) cell, a dendritic cell, a human embryonic stem cell, a mesenchymal stem cell, a hematopoietic stem cell, dendritic cell, a macrophage, a myeloid cell, a B cell, a platelet, an endothelial cell, an epithelial cell, and a fibroblast.

The present disclosure also provides methods for activating dendritic cells in a subject. The methods may include the steps of administering to the subject a therapeutically effective amount of immune cells. The immune cells may include or express a composition comprising a stimulus response element (SRE) operably linked to a first payload. In one embodiment, the immune cell is a T cell. The first payload may include, in whole or in part, human CD40L (SEQ ID NO: 3820), or a CD40L mutant as described herein. The immune cell may also express a pharmaceutical composition that includes the compositions described herein. The methods also involve administering to the subject a therapeutically effective amount of a stimulus. In one embodiment, the stimulus is a ligand. The methods further may include measuring the dendritic cell activation marker IL12 in the subject in response to the ligand to determine dendritic cell activation. In one embodiment, the dendritic cell may be a myeloid dendritic cell, a plasmacytoid dendritic cell, a CD14+ dendritic cell, a Langerhans cell, or a microglia. In one aspect, the dendritic cell is a myeloid dendritic cell.

In one embodiment, the CD40L or mutant thereof, immunotherapeutic agent may be derived from UniProt ID: P29965 (also referred to herein as the “WT”). The payloads of the present disclosure may be a region or portion of CD40L, with or without a mutation in the amino acid or nucleotide sequence encoding such mutant. Non limiting examples of regions of CD40L include but are not limited to amino acids 113-269 of UniProt ID: P2996, wherein the cytoplasmic domain, the transmembrane domain and a portion of the extracellular domain have been removed from UniProt ID: P2996 leaving a portion of the extracellular domain and the receptor binding domain intact. In one embodiment, the payload may be amino acids 14-261 of UniProt ID: P2996, which excludes the cytoplasmic tail of CD40L, thereby may potentially reduce internalization. In one aspect, the payload may be amino acids 14-261 of UniProt ID: P2996 with a deletion in amino acids S110-G116, which renders the CD40L resistant to cleavage by proteolytic enzymes.

In some embodiments, the mutations may be engineered within CD40L payload such that it does not to bind to or bind with reduced affinity to CD40L endogenously expressed by cells described herein. CD40L is a type II transmembrane protein that forms a trimer on the cell surface. In some embodiments, trimerization occurs through the interaction of amino acid residues 47-261 of SEQ ID NO: 3820. In some embodiments, residues within 47-261 of SEQ ID NO: 3820 may be mutated in the CD40L payload to prevent trimerization (herein referred to as “trimerization mutants.” In some embodiments the residues within 116-261 of SEQ ID NO: 3820 may be mutated. In some aspects, mutations may allow selective trimerization such that a CD40L trimerization mutant may be able to bind to another CD40L trimerization mutant protein but not to a CD40L protein lacking the mutations. Trimerization mutations sites may be sites within the CD40L protein that are involved in the trimerization as determined by the crystal structure of the CD40L trimer. Positions within CD40L that may be mutated include but are not limited to amino acids at position 125, 170, 172, 224, 226 and/or 227 of SEQ ID NO: 3820. In some embodiments, the mutations to CD40L payload to prevent its trimerization with the endogenous CD40L may include but are not limited to Y170G, Y172G, H224G, G226F, G226H, G226W, and/or G227F.

Sheddases e.g. ADAM10/17 present in the tumor microenvironment can cleave CD40L thereby preventing the successful activation of CD40 by CD40L. Analysis of the sequence of CD40L reveals an ADAM10/17 proteolytic cleavage site. In some embodiments, a deletion of amino acids 1-13 of CD40L may be engineered to reduce internalization. A deletion of amino acids 110-116 of CD40L may also be designed to remove the ADAM10/17 sites. Deletion or mutation of the methionine residue at amino acid position 113 of CD40L may also be utilized to reduce cleave by ADAM10/17 enzymes. In one embodiment, a region or portion of the human CD40L protein may be replaced by the murine CD40L protein sequence to generate a CD40L protein that is resistant to cleavage by ADAM10/17. Any of the CD40L sequences aimed at reducing its shedding as described in US Patent Publication US20180085451A1 and/or U.S. Pat. No. 7,495,090B2 may be used in the effector modules and biocircuits described herein (the contents of each of which are incorporated by reference in their entirety). CD40L may be tethered to the membrane using any of the transmembrane domains. In one embodiment, CD40L may be tethered to the membrane using CD8 derived domains such as but not limited to CD8 transmembrane domain, CD8 hinge domain and/or CD8 cytoplasmic tail.

In some embodiments the effector modules described herein may include one or more cleavage sites between DD and CD40L. Inclusion of cleavage sites may uncouple the proteolytic turnover of the DD from the payload, thereby altering the levels of expression of the payload independent of the DD. In some embodiments, the addition of the cleavage site my increase expression of the payload. In other aspects, addition of cleavage site may reduce the expression of the payload.

In some embodiments, the CD40L payload and the SREs described herein may linked.

CD40L construct components and CD40L constructs are provided in Table 7 and Table 8 respectively. In Table 7 and Table 8, CD40L “WT” refers to Uniprot ID: P29965, hPDE5 “WT” refers to Uniprot ID: 076074 and ER “WT” refers to Uniprot ID: P03372.2.

TABLE 7
CD40L construct components

| | | | NA |
|---|---|---|---|
| | | AA | Se- |
| | | SEQ | quence |
| | | ID | or SEQ |
| Description | AA sequence | NO: | ID NO: |
| Linker (MH) | MH | — | ATGCAT |
| Flexible | GS | — | GGATCC, |
| G/S rich | | | GGATCT |
| linker; | | | GGATCA |
| BamHI Site | | | |
| Linker (SG) | SG | — | TCAGGG |
| Linker (EF) | EF | — | GAATTC |
| Linker | GSSG | 3814 | 3816 |
| (GSSG) | | | |
| Linker | GGSGGGSGGGSG | 6532 | 6533; |
| (GGSGGG | | | 6594- |
| SGGGSG) | | — | 6597 |
| Linker (H) | H | — | CAT |
| Flexible | GS | | GGTTCC, |
| G/S rich | | | GGATCCG |
| linker; | | | GTTCA, |
| BamH1 Site | | | GGATCTG |
| | | | GATCA, |
| | | | GGTAGT |
| Linker (GSG) | GSG | — | GGATC |
| (BamHI-Gly) | | | CGGA, |
| | | | GGATC |
| | | | CGGT, |
| | | | GGATC |
| | | | TGGT; |
| IL2 Signal | MYRMQLLSCIALSLALV | 1230 | 1234; |
| sequence | TNS | | 3817; |
| | | | 3818 |
| CD8α leader | MALPVTALLLPLALLLHA | 870 | 871 |
| | ARP | | |
| ecDHFR | ISLIAALAVDYVIGMENAM | 255 | 263 |
| (aa 2-159 of | PWNLPADLAWFKRNTLNKP | | |
| WT, R12Y, | RVIMGHTWESIGRPLP | | |
| Y100I) | GRKNIILSSQPGTDDRVTWV | | |
| | KSVDEAIAACGDVPEIMVIG | | |
| | GGRVIEQFLPKAQKLYLTHI | | |
| | DAEVEGDTHFPDYEPDDWES | | |
| | VFSEFHDADAQNSHSYCFEI | | |
| | LERR | | |
| FKBP | GVQVETISPGDGRTFPKRGQ | 277 | 3819 |
| (2-108 | TCVVHYTGMLEDGKKVDSSR | | |
| of WT, | DRNKPFKFMLGKQEVIRGWE | | |
| F37V, | EGVAQMSVGQRAKLTISPDY | | |
| L107P) | AYGATGHPGIIPPHATLVFD | | |
| | VELLKPE | | |
| hPDE5 | EETRELQSLAAAVVPSAQTL | 308 | 309-312 |
| (aa 535-860 | KITDFSFSDFELSDLETALC | | |
| of | TIRMFTDLNLVQNFQMKHEV | | |
| WT, R732L) | LCRWILSVKKNYRKNVAYHN | | |
| | WRHAFNTAQCMFAALKAGKI | | |
| | QNKLTDLEILALLIAALSHD | | |
| | LDHRGVNNSYIQRSEHPLAQ | | |
| | LYCHSIMEHHHFDQCLMILN | | |
| | SPGNQILSGLSIEEYKTTLK | | |
| | IIKQAILATDLALYIKRLGE | | |
| | FFELIRKNQFNLEDPHQKEL | | |
| | FLAMLMTACDLSAITKPWPI | | |
| | QQRIAELVATEFFDQGDRER | | |
| | KELNIEPTDLMNREKKNKIP | | |
| | SMQVGFIDAICLQLYEALTH | | |
| | VSEDCFPLLDGCRKNRQKWQ | | |
| | ALAEQQ | | |
| hPDE5 | EETRELOSLAAAVVPSAQTL | 560 | 561-562 |
| (aa 535-860 | KITDFSFSDFELSDLETALC | | |
| of | TIRMFTDLNLVQNFQMKHEV | | |
| WT, R732L, | LCRWILSVKKNYRKNVAYHN | | |
| F736A) | WRHAFNTAQCMFAALKAGKI | | |
| | QNKLTDLEILALLIAALSHD | | |
| | LDHRGVNNSYIQRSEHPLAQ | | |
| | LYCHSIMEHHHFDQCLMILN | | |
| | SPGNQILSGLSIEEYKTTLK | | |
| | IIKQAILATDLALYIKRLGE | | |
| | FAELIRKNQFNLEDPHQKEL | | |
| | FLAMLMTACDLSAITKPWPI | | |
| | QQRIAELVATEFFDQGDRER | | |
| | KELNIEPTDLMNREKKNKIP | | |
| | SMQVGFIDAICLQLYEALTH | | |
| | VSEDCFPLLDGCRKNRQKWQ | | |
| | ALAEQQ | | |
| PDE5 | EETRELQSLAAAVVPSAQTL | 563 | 564 |
| (aa 535-860 | KITDFSFSDFELSDLETALC | | |
| of | TIRMFTDLNLVQNFQMKHEV | | |
| WT, H653A, | LCRWILSVKKNYRKNVAYHN | | |
| R732L) | WRHAFNTAQCMFAALKAGKI | | |
| | QNKLTDLEILALLIAALSAD | | |
| | LDHRGVNNSYIQRSEHPLAQ | | |
| | LYCHSIMEHHHFDQCLMILN | | |
| | SPGNQILSGLSIEEYKTTLK | | |
| | IIKQAILATDLALYIKRLGE | | |
| | FFELIRKNQFNLEDPHQKEL | | |
| | FLAMLMTACDLSAITKPWPI | | |
| | QQRIAELVATEFFDQGDRER | | |
| | KELNIEPTDLMNREKKNKIP | | |
| | SMQVGFIDAICLQLYEALTH | | |
| | VSEDCFPLLDGCRKNRQKWQ | | |
| | ALAEQQ | | |
| ER | SLALSLTADQMVSALLDAEP | 633 | 636 |
| (aa | PILYSEYDPTRPFSEASMMG | | |
| 305-549 of | LLTNLADRELVHMINWAKRV | | |
| WT, | PGFVDLALHDQVHLLECAWM | | |
| T371A, | EILMIGLVWRSMEHPGKLLF | | |
| L384M, | APNLLLDRNQGKCVEGGVEI | | |
| M421G, | FDMLLATSSRFRMMNLQGEE | | |
| N519S, | FVCLKSULLNSGVYTFLSST | | |
| G521R, | LKSLEEKDHIHRVLDKITDT | | |
| Y537S) | LIHLMAKAGLTLQQQHQRLA | | |
| | QLLLILSHIRHMSSKRMEHL | | |
| | YSMKCKNVVPLSDLLLEMLD | | |
| | AHRL | | |
| ER | SLALSLTADQMVSALLDAEP | 648 | 649 |
| (aa 305-549 | PILYSEYDPTRPFSEASMMG | | |
| of | LLTNLADRELVHMINWAKRV | | |
| WT, | PGFVDLTLHDQVHLLECAWM | | |
| L384M, | EILMIGLVWRSMEHPGKLLF | | |
| N413D, | APNLLLDRDQGKCVEGGVEI | | |
| M421G, | FDMLLATSSRFRMMNLQGEE | | |
| G521R, | FVCLKSULLNSGVYTFLSST | | |
| Y537S) | LKSLEEKDHIHRVLDKITDT | | |
| | LIHLMAKAGLTLQQQHQRLA | | |
| | QLLLILSHIRHMSNKRMEHL | | |
| | YSMKCKNVVPLSDLLLEMLD | | |
| | AHRL | | |
| hDHFR | MVGSLNCIVAVSQNMGIGKN | 6548 | 6549 |
| (Q36E, | GDLPWPPLRNEFRYFERMTT | | |
| QI03H, | TSSVEGKQNLVIMGKKTWFS | | |
| Y122I) | IPEKNRPLKGRINLVLSREL | | |
| | KEPPQGAHFLSRSLDDALKL | | |
| | TEHPELANKVDMVWIVGGSS | | |
| | VIKEAMNHPGHLKLF | | |
| | VTRIMQDFESDTFFPEIDLE | | |
| | KYKLLPEYPGVLSDVQEEKG | | |
| | IKYKFEVYEKND | | |
| hDHFR (aa | VGSLNCIVAVSQNMGIGKNG | 145 | 146-148 |
| 2-187 of | DLPWPPLRNEFRYFQRMTTT | | |
| WT, Y122I) | SSVEGKQNLVIMGKKTWFSI | | |
| | PEKNRPLKGRINLVLSRELK | | |
| | EPPQGAHFLSRSLDDALKLT | | |
| | EQPELANKVDMVWIVGGSSV | | |
| | IKEAMNHPGHLKLFVTRIMQ | | |
| | DFESDTFFPEIDLEKYKLLP | | |
| | EYPGVLSDVQEEKGIKYKFE | | |
| | VYEKND | | |
| hDHFR | VGSLNCIVAVSQNMGIGKNG | 6552 | 6553 |
| (aa 2-187 | DLPWPPLRNEFRYFQRMTTT | | |
| of | SSVEGKQNLVIMGRKTWFSI | | |
| WT, K55R, | PEKKRPLKGRINLVLSRELK | | |
| N65K, | EPPQGAHFLSRSLDDALKLT | | |
| Y1221) | EQPELANKVDMVWIVGGSSV | | |
| | IKEAMNHPGHLKLFVTRIMQ | | |
| | DFESDTFFPEIDLEKYKLLP | | |
| | EYPGVLSDVQEEKGIKYKFE | | |
| | VYEKND | | |
| CD40L | MIETYNQTSPRSAATGLPIS | 3820 | 3821 |
| (UniProt | MKIFMYLLTVFLITQMIGSA | | |
| ID: | LFAVYLHRRLDKIEDERNLH | | |
| P29965) | EDFVFMKTIQRCNTGERSLS | | |
| | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | QSIHLGGVFELQPGASVFVN | | |
| | VTDPSQVSHGTGFTSFGLLK | | |
| | L | | |
| sCD40L | MQKGDQNPQIAAHVISEASS | 3822 | 3823 |
| (113-269 | KTTSVLQWAEKGYYTMSNNL | | |
| Of WT) | VTLENGKQLTVKRQGLYYIY | | |
| | AQVTFCSNREASSQAPFIAS | | |
| | LCLKSPGRFERILLRAANTH | | |
| | SSAKPCGQQSIHLGGVFELQ | | |
| | PGASVFVNVTDPSQVSHGTG | | |
| | FTSFGLLKL | | |
| CD40L | ATGLPISMKIFMYLLTVFLI | 3824 | 3825 |
| (aa 14-261 | TQMIGSALFAVYLHRRLDKI | | |
| Of WT) | EDERNLHEDFVFMKTIQRCN | | |
| | TGERSLSLLNCEEIKSQFEG | | |
| | FVKD | | |
| | IMLNKEETKKENSFEMQKGD | | |
| | QNPQIAAHVISEASSKTTSV | | |
| | LQWAEKGYYTMSNNLVTLEN | | |
| | GKQLTVKRQGLYYIYAQVTF | | |
| | CSNREASSQAPFIASLCLKS | | |
| | PGRFERILLRAANTHSSAKP | | |
| | CGQQSIHLGGVFELQPGASV | | |
| | VFNVTDPSQVSHGTGFTSFG | | |
| | LLKL | | |
| CD40L | MIETYNQTSPRSAATGLPIS | 3826 | 3827 |
| (aa 1-261 | MKIFMYLLTVFLITQMIGSA | | |
| of | LFAVYLHRRLDKIEDERNLH | | |
| WT, (S110- | EDFVFMKTIQRCNTGERSLS | | |
| 0116) del) | LLNCEEIKSQFEGFVKDIML | | |
| | NKEETKKENDQNPQIAAHVI | | |
| | SEASSKTTSVLQWAEKGYYT | | |
| | MSNNLVTLENGKQLTVKRQG | | |
| | LYYIYAQVTFCSNREASSQA | | |
| | PFIASLCLKSPGRFERILLR | | |
| | AANTHSSAKPCGQQSIHLGG | | |
| | VFELQPGASVFVNVTDPSQV | | |
| | SHGTGFTSFGLLKL | | |
| CD40L | MIETYNQTSPRSAATGLPIS | 6598 | 6599 |
| (H224G, | MKIFMYLLTVFLITQMIGSA | | |
| G226F) | LFAVYLHRRLDKIEDERNLH | | |
| | EDFVFMKTIQRCNTGERSLS | | |
| | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | QSIGLFGVFELQPGASVFVN | | |
| | VTDPSQVSHGTGFTSFGLLK | | |
| | L | | |
| CD40L | MIETYNQTSPRSAATGLPIS | 6600 | 6601 |
| (H224G, | MKIFMYLLTVFLITQMIGSA | | |
| G226H) | LFAVYLHRRLDKIEDERNLH | | |
| | EDFVFMKTIQRCNTGERSLS | | |
| | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | Q | | |
| | SIGLHGVFELQPGASVFVNV | | |
| | TDPSQVSHGTGFTSFGLLKL | | |
| CD40L | MIETYNQTSPRSAATGLPIS | 6602 | 6603 |
| (Y172G, | MKIFMYLLTVFLITQMIGSA | | |
| G226F) | LFAVYLHRRLDKIEDERNLH | | |
| | EDFVFMKTIQRCNTGERSLS | | |
| | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYYIGAQVTFCSN | | |
| | REASSQAPFIASLCLKSPGR | | |
| | FERILLRAANTHSSAKPCGQ | | |
| | QSIHLFGVFELQPGASVFVN | | |
| | VTDPSQVSHGTGFTSFGLLK | | |
| | L | | |
| CD40L | MIETYNQTSPRSAATGLPIS | 6604 | 6605 |
| (Y170G, | MKIFMYLLTVFLITQMIGSA | | |
| H224G, | LFAVYLHRRLDKIEDERNLH | | |
| G226W) | EDFVFMKTIQRCNTGERSLS | | |
| | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYGIYAQVTFCSN | | |
| | REASSQAPFIASLCLKSPGR | | |
| | FERILLRAANTHSSAKPCGQ | | |
| | QSIGLWGVFELQPGASVFVN | | |
| | VTDPSQVSHGTGFTSFGLLK | | |
| | L | | |
| CD40L | MIETYNQTSPRSAATGLPIS | 6606 | 6607 |
| (H125G, | MKIFMYLLTVFLITQMIGSA | | |
| G227F) | LFAVYLHRRLDKIEDERNLH | | |
| | EDFVFMKTIQRCNTGERSLS | | |
| | LLNCEEIKSQFEGFVKDIML | | |
| | NKEETKKENSFEMQKGDQNP | | |
| | QIAAGVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | QSIHLGFVFELQPGASVFVN | | |
| | VTDPSQVSHGTGFTSFGLLK | | |
| | L | | |
| CD40L | MIETYNQTSPRSAATGLPIS | 6674 | 6675 |
| (S110G, | MKIFMYLLTVFLITQMIGSA | | |
| F111G, | LFAVYLHRRLDKIEDERNLH | | |
| E112S, | EDFVFMKTIQRCNTGERSLS | | |
| M113G, | LLNC | | |
| Q1I4G, | EEIKSQFEGFVKDIMLNKEE | | |
| K115S) | TKKENGGSGGSGDQNPQIAA | | |
| | HVISEASSKTTSVLQWAEKG | | |
| | YYTMSNNLVTLENGKQLTVK | | |
| | RQGLYYIYAQVTFCSNREAS | | |
| | SQAPFIASLCLKSPGRFERI | | |
| | LLRAANTHSSAKPCGQQSIH | | |
| | LGGVFELQPGASVFVNVTDP | | |
| | SQVSHGTGFTSFGLLKL | | |
| CD19 scFv | DIQMTQTTSSLSASLGDRVT | 4049 | 4055 |
| | ISCRASQDISKYLNWYQQKP | | |
| | DGTVKLLIYHTSRLHSGVPS | | |
| | RFSGSGSGTDYSLTISNLEQ | | |
| | EDIATYFCQQGNTLPYTFGG | | |
| | GTKLEITGGGGSGGGGSGGG | | |
| | VKGSELQESGPGLVAPSQSL | | |
| | SVTCTVSGVSLPDYGVSWIR | | |
| | RQPPKGLEWLGVIWGSETTY | | |
| | ALYNSKSRLTIIKDNSKSQV | | |
| | MFLKNSLQTDDTAIYYCAKH | | |
| | YYYGGSYAMDYWGQGTSVTV | | |
| | SS | | |
| CD8a | TTTPAPRPPTPAPTIASQPL | 4866 | 4868 |
| Hinge and | SLRPEACRPAAGGAVHTRGL | | |
| Transmembrane | DFACDIYIWAPLAGTCGVLL | | |
| Domain | LSLVITLYC | | |
| CD28 co- | KRGRKKLLYIFKQPFMRPVQ | 5103 | 5110 |
| stimulatory | TTQEEDGCSCRFPEEEEGGC | | |
| domain; | EL | | |
| 4-1BB | | | |
| intracellular | | | |
| domain | | | |
| CD3 zeta | RVKFSRSADAPAYKQGQNQL | 4990 | 4996 |
| intracellular | YNELNLGRREEYDVLDKRRG | | |
| domain | RDPEMGGKPRRKNPQEGLYN | | |
| | ELQKDKMAEAYSEIGMKGER | | |
| | RRGKGHDGLYQGLSTATKDT | | |
| | YDALHMQALPPR | | |
| P2A cleavage | ATNFSLLKQAGDVEENPGP | 1637 | 1638 |
| site | | | |
| Spacer 7 | — | — | 6536 |
| Spacer 6 | — | — | 6537 |
| IRES | — | — | 6538 |
| CD8 | NHRNRR | 6608 | 6609 |
| Cytoplasmic | | | |
| tail | | | |

TABLE 8
CD40L constructs

| | | AA | NA |
|---|---|---|---|
| | | SEQ | SEQ |
| Construct Name | | ID | ID |
| (Description) | AA sequence | NO: | NO: |
| OT-001661 (MH | MHMIETYNQTSPRSAATGLP | 3828 | 3829 |
| Linker; | ISMKIFMYLLTVFLITQMIG | | |
| CD40L; stop) | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-001685 | MGVQVETISPGDGRTFPKRG | 3830 | 3831 |
| (Met; | QTCVVHYTGMLEDGKKVDSS | | |
| FKBP | RDRNKPFKFMLGKQEVIRGW | | |
| (M1del, F37V, | EEGVAQMSVGQRAKLTISPD | | |
| L107P); | YAYGATGHPGIIPPHATLVF | | |
| Flexible G/S | DVELLKPEGSMHMIETYNQT | | |
| rich linker; | SPRSAATGLPISMKIFMYLL | | |
| MH Linker; | TVFLITQMIGSALFAVYLHR | | |
| CD40L; stop) | RLDKIEDERNLHEDFVFMKT | | |
| | IQRCNTGERSLSLLNCEEIK | | |
| | SQFEGFVKDIMLNKEETKKE | | |
| | NSFEMQKGDQNPQIAAHVIS | | |
| | EASSKTTSVLQWAEKGYYTM | | |
| | SNNLVTLENGKQLTVKRQGL | | |
| | YYIYAQVTFCSNREASSQAP | | |
| | FIASLCLKSPGRFERILLRA | | |
| | ANTHSSAKPCGQQSIHLGGV | | |
| | FELQPGASVFVNVTDPSQVS | | |
| | HGTGFTSFGLLKL* | | |
| OT-001662 (Met; | MSGISLIAALAVDYVIGMEN | 3832 | 3833 |
| Linker (SG); | AMPWNLPADLAWFKRNTLNK | | |
| ecDHFR (M1del, | PVIMGRHTWESIGRPLPGRK | | |
| RI2Y, Y100I); | NIILSSQPGTDDRVTWVKSV | | |
| Histidine | DEAIAACGDVPEIMVIGGGR | | |
| residue; | VIEQFLPKAQKLYLTHIDAE | | |
| CD40L; stop) | VEGDTHFPDYEPDDWESVFS | | |
| | EFHDADAQNSHSYCFEILER | | |
| | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAK | | |
| | PCGQQSIHLGGVFELQPGAS | | |
| | VFVNVTDPSQVSHGTGFTSF | | |
| | GLLKL* | | |
| OT-001666 | MSLALSLTADQMVSALLDAE | 3834 | 3835 |
| (Met; ER | PPILYSEYDPTRPFSEASMM | | |
| (305-549 | GLLTNLADRELVHMINWAKR | | |
| of WT, | VPGFVDLTLHDQVHLLECAW | | |
| L384M, N413D, | MEILMIGLVWRSMEHPGKLL | | |
| M42IG, G521R, | FAPNLLLDRDQGKCVEGGVE | | |
| Y537S); | IFDMLLATSSRFRMMNLQGE | | |
| Histidine; | EFVCLKSIILLNSGVYTFLS | | |
| CD40L; stop) | STLKSLEEKDHIHRVLDKIT | | |
| | DTLIHLMAKAGLTLQQQHQR | | |
| | LAQLLLILSHIRHMSNKRME | | |
| | HLYSMKCKNVVPLSDLLLEM | | |
| | LDAHRLHMIETYNQTSPRSA | | |
| | ATGLPISMKIFMYLLTVFLI | | |
| | TQMIGSALFAVYLHRRLDKI | | |
| | EDERNLHEDFVFMKTIQRCN | | |
| | TGERSLSLLNCEEIKSQFEG | | |
| | FVKDIMLNKEETKKENSFEM | | |
| | QKGDQNPQIAAHVISEASSK | | |
| | TTSVLQWAEKGYYTMSNNLV | | |
| | TLENGKQLTVKRQGLYYIYA | | |
| | QVTFCSNREASSQAPFIASL | | |
| | CLKSPGRFERILLRAANTHS | | |
| | SAKPCGQQSIHLGGVFELQP | | |
| | GASVFVNVTDPSQVSHGTGF | | |
| | TSFGLLKL* | | |
| OT-001667 | MSLALSLTADQMVSALLDAE | 3836 | 3837 |
| (Met; ER | PPILYSEYDPTRPFSEASMM | | |
| (aa 305-549 | GLLTNLADRELVHMINWAKR | | |
| of WT, | VPGFVDLALHDQVHLLECAW | | |
| T371A, L384M, | MEILMIGLVWRSMEHPGKLL | | |
| M421G, N519S, | FAPNLLLDRNQGKCVEGGVE | | |
| G52IR, Y537S); | IFDMLLATSSRFRMMNLQGE | | |
| Histidine; | EFVCLKSIILLNSGVYTFLS | | |
| CD40L; | STLKSLEEKDHIHRVLDKIT | | |
| stop) | DTLIHLMAKAGLTLQQQHQR | | |
| | LAQLLLILSHIRHMSSKRME | | |
| | HLYSMKCKNVVPLSDLLLEM | | |
| | LDAHRLHMIETYNQTSPRSA | | |
| | ATGLPISMKIFMYLLTVFLI | | |
| | TQMIGSALFAVYLHRRLDKI | | |
| | EDERNLHEDFVFMKTIQRCN | | |
| | TGERSLSLLNCEEIKSQFEG | | |
| | FVKDIMLNKEETKKENSFEM | | |
| | QKGDQNPQIAAHVISEASSK | | |
| | TTSVLQWAEKGYYTMSNNLV | | |
| | TLENGKQLTVKRQGLYYIYA | | |
| | QVTFCSNREASSQAPFIASL | | |
| | CLKSPGRFERILLRAANTHS | | |
| | SAKPCGQQSIHLGGVFELQP | | |
| | GASVFVNVTDPSQVSHGTGF | | |
| | TSFGLLKL* | | |
| OT-001672 (IL2 | MYRMQLLSCIALSLALVTNS | 3838 | 3839 |
| signal | GSMHMQKGDQNPQIAAHVI | | |
| sequence; | SEASSKTTSVLQWAE | | |
| Flexible | KGYYTMSNNLVTLENGKQLT | | |
| G/S rich | VKRQGLYYIYAQVTFCSNRE | | |
| linker; | ASSQAPFIASLCLKSPGRFE | | |
| MH Linker; | RILLRAANTHSSAKPCGQQS | | |
| CD40L | IHLGGVFELQPGASVFVNVT | | |
| (aa 113-269 | DPSQVSHGTGFTSFGLLKL* | | |
| of WT); | | | |
| stop) | | | |
| OT-001686 (IL2 | MYRMQLLSCIALSLALVTNS | 3840 | 3841 |
| signal | EFGVQVETISPGDGRTFPKR | | |
| sequence; | GQTCVVHYTGMLEDGKKVDS | | |
| Linker | SRDRNKPFKFMLGKQEVIRG | | |
| (EF); FKBP | WEEGVAQMSVGQRAKLTISP | | |
| (M1del, F37V, | DYAYGATGHPGIIPPHATLV | | |
| L107P); | FDVELLKPEGSMHMQKGDQN | | |
| Flexible G/S | PQIAAHVISEASSKTTSVLQ | | |
| rich linker; | WAEKGYYTMSNNLVTLENGK | | |
| MH Linker; | QLTVKRQGLYYIYAQVTFCS | | |
| CD40L (aa | NREASSQAPFIASLCLKSPG | | |
| 113-269 | RFERILLRAANTHSSAKPCG | | |
| of WT); | QQSIHLGGVFELQPGASVFV | | |
| stop) | NVTDPSQVSHGTGFTSFGLL | | |
| | KL* | | |
| OT-001673 | MYRMQLLSCIALSLALVTNS | 3842 | 3843 |
| (IL2 | GSSGISLIAALAVDYVIGME | | |
| signal | NAMPWNLPADLAWFKRNTLN | | |
| sequence; | KPVIMGRHTWESIGRPLPGR | | |
| Linker | KNIILSSQPGTDDRVTWVKS | | |
| (GSSG); | VDEAIAACGDVPEIMVIGGG | | |
| ccDHFR (Mldel, | RVIEQFLPKAQKLYLTHIDA | | |
| R12Y, | EVEGDTHFPDYEPDDWESVF | | |
| Y100I); | SEFHDADAQNSHSYCFEILE | | |
| Flexible | RRGSMHMQKGDQNPQIAAHV | | |
| G/S rich | ISEASSKTTSVLQWAEKGYY | | |
| linker; | TMSNNLVTLENGKQLTVKRQ | | |
| MH Linker; | GLYYIYAQVTFCSNREASSQ | | |
| CD40L | APFIASLCLKSPGRFERILL | | |
| (aa 113-269 | RAANTHSSAKPCGQQSIHLG | | |
| of WT); | GVFELQPGASVFVNVTDPSQ | | |
| stop) | VSHGTGFTSFGLLKL* | | |
| OT-001674 | MYRMQLLSCIALSLALVTNS | 3844 | 3845 |
| (IL2 | GSEETRELQSLAAAVVPSAQ | | |
| signal | TLKITDFSFSDFELSDLETA | | |
| sequence; | LCTIRMFTDLNLVQNFQMKH | | |
| Flexible | EVLCRWILSVKKNYRKNVAY | | |
| G/S rich | HNWRHAFNTAQCMFAALKAG | | |
| linker; | KIQNKLTDLEILALLIAALS | | |
| hPDE5(aa. | ADLDHRGVNNSYIQRSEHPL | | |
| 535 to 860 | AQLYCHSIMEHHHFDQCLMI | | |
| of WT. | LNSPGNQILSGLSIEEYKTT | | |
| H653A, | LKIIKQAILATDLALYIKRL | | |
| R732L); | GEFFELIRKNQFNLEDPHQK | | |
| Flexible | ELFLAMLMTACDLSAITKPW | | |
| G/S rich | PIQQRIAELVATEFFDQGDR | | |
| linker; | ERKELNIEPTDLMNREKKNK | | |
| MH Linker; | IPSMQVGFIDAICLQLYEAL | | |
| CD40L | THVSEDCFPLLDGCRKNRQK | | |
| (aa 113-269 | WQAL | | |
| of WT); stop) | AEQQGSMHMQKGDQNPQIAA | | |
| | HVISEASSKTTSVLQWAEKG | | |
| | YYTMSNNLVTLENGKQLTVK | | |
| | RQGLYYIYAQVTFCSNREAS | | |
| | SQAPFIASLCLKSPGRFERI | | |
| | LLRAANTHSSAKPCGQQSIH | | |
| | LGGVFELQPGASVFVNVTDP | | |
| | SQVSHGTGFTSFGLLKL* | | |
| OT-001675 (IL2 | MYRMQLLSCIALSLALVTNS | 3846 | 3847 |
| signal | GSEETRELQSLAAAVVPSAQ | | |
| sequence; | TLKITDFSFSDFELSDLETA | | |
| Flexible | LCTIRMFTDLNLVQNFQMKH | | |
| G/S rich | EVLCRWILSVKKNYRKNVAY | | |
| linker; | HNWRHAFNTAQCMFAALKAG | | |
| hPDE5 (aa | KIQNKLTDLEILALLIAALS | | |
| 535-860 | HDLDHRGVNNSYIQRSEHPL | | |
| of WT, | AQLYCHSIMEHHHFDQCLMI | | |
| R732L, | LNSPGNQILSGLSIEEYKTT | | |
| F736A); | LKIIKQAILATDLALYIKRL | | |
| Flexible | GEFAELIRKNQFNLEDPHQK | | |
| G/S rich | ELFLAMLMTACDLSAITKPW | | |
| linker; | PIQQRIAELVATEFFDQGDR | | |
| MH Linker; | ERKELNIEPTDLMNREKKNK | | |
| CD40L | IPSMQVGFIDAICLQLYEAL | | |
| (aa 113-269 | THVSEDCFPLLDGCRKNRQK | | |
| of WT); stop) | WQALAEQQGSMHMQKGDQNP | | |
| | QIAAHVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | QSIHLGGVFELQPGASVFVN | | |
| | VTDPSQVSHGTGFTSFGLLK | | |
| | L* | | |
| OT-001676 | MYRMQLLSCIALSLALVTNS | 3848 | 3849 |
| (IL2 | GSEETRELQSLAAAVVPSAQ | | |
| signal | TLKITDFSFSDFELSDLETA | | |
| sequence; | LCTIRMFTDLNLVQNFQMKH | | |
| Flexible | EVLCRWILSVKKNYRKNVAY | | |
| G/S rich | HNWRHAFNTAQCMFAALKAG | | |
| linker; | KIQNKLTDLEILALLIAALS | | |
| hPDE5 (aa | HDLDHRGVNNSYIQRSEHPL | | |
| 535-860 of WT, | AQLYCHSIMEHHHFDQCLMI | | |
| R732L); | LNSPGNQILSGLSIEEYKTT | | |
| Flexible | LKIIKQAILATDLALYIKRL | | |
| G/S | GEFFELIRKNQFNLEDPHQK | | |
| rich linker; | ELFLAMLMTACDLSAITKPW | | |
| MH Linker; | PIQQRIAELVATEFFDQGDR | | |
| CD40L (aa | ERKELNIEPTDLMNREKKNK | | |
| 113-269 of WT); | IPSMQVGFIDAICLQLYEA | | |
| stop) | LTHVSEDCFPLLD | | |
| | GCRKNRQKWQALAEQQGSMH | | |
| | MQKGDQNPQIAAHVISEASS | | |
| | KTTSVLQWAEKGYYTMSNNL | | |
| | VTLENGKQLTVKRQGLYYIY | | |
| | AQVTFCSNREASSQAPFIAS | | |
| | LCLKSPGRFERILLR | | |
| | AANTHSSAKPCGQQSIHLGG | | |
| | VFELQPGASVFVNVTDPSQV | | |
| | SHGTGFTSFGLLKL* | | |
| OT-001677 | MYRMQLLSCIALSLALVTNS | 3850 | 3851 |
| (IL2 | GSSLALSLTADQMVSALLDA | | |
| signal | EPPILYSEYDPTRPFSEASM | | |
| sequence; | MGLLTNLADRELVHMINWAK | | |
| Flexible | RVPGFVDLTLHDQVHLLECA | | |
| G/S rich | WMEILMIGLVWRSMEHPGKL | | |
| linker; | LFAPNLLLDRDQGKCVEGGV | | |
| ER (305-549 | EIFDMLLATSSRFRMMNLQG | | |
| of WT, | EEFVCLKSIILLNSGVYTFL | | |
| L384M, | SSTLKSLEEKDHIHRVLDKI | | |
| N413D, | TDTLIHLMAKAGLTLQQQHQ | | |
| M421G, | RLAQLLLILSHIRHMSNKRM | | |
| G52IR, | EHLYSMKCKNVVPLSDLLLE | | |
| Y537S); | MLDAHRLGSMHMQKGDQNPQ | | |
| Flexible | IAAHVISEASSKTTSVLQWA | | |
| G/S rich | EKGYYTMSNNLVTLENGKQL | | |
| linker; | TVKRQGLYYIYAQVTFCSNR | | |
| MH Linker; | EASSQAPFIASLCLKSPGRF | | |
| CD40L | ERILLRAANTHSSAKPCGQQ | | |
| (aa 113-269 | SIHLGGVFELQPGASVFVNV | | |
| of WT); | TDPSQVSHGTGFTSFGLLKL | | |
| stop)) | * | | |
| OT-001684 (IL2 | MYRMQLLSCIALSLALVTNS | 3852 | 3853 |
| signal | GSSLALSLTADQMVSALLDA | | |
| sequence; | EPPILYSEYDPTRPFSEASM | | |
| Flexible | MGLLTNLADRELVHMINWAK | | |
| G/S rich | RVPGFVDLALHDQVHLLECA | | |
| linker; ER | WMEILMIGLVWRSMEHPGKL | | |
| (aa 305-549 | LFAPNLLLDRNQGKCVEGGV | | |
| Of WT, T371A, | EIFDMLLATSSRFRMMNLQG | | |
| L384M, M421G, | EEFVCLKSULLNSGVYTFLS | | |
| N519S, G521R, | STLKSLEEKDHIHRVLDKIT | | |
| Y537S); | DTLIHLMAKAGLTLQQQHQR | | |
| Flexible G/S | LAQLLLILSHIRHMSSKRME | | |
| rich linker; | HLYSMKCKNVVPLSDLLLEM | | |
| MH Linker; | LDAHRLGSMHMQKGDQNPQI | | |
| CD40L (aa | AAHVISEASSKTTSVLQWAE | | |
| 113-269 | KGYYTMSNNLVTLENGKQLT | | |
| of WT); | VKRQGLYYIYAQVTFCSNRE | | |
| stop) | ASSQAPFIASLCLKSPGRFE | | |
| | RILLRAANTHSSAKPCGQQS | | |
| | IHLGGVFELQPGASVFVNVT | | |
| | DPSQVSHGTGFTSFGLLKL* | | |
| OT-001669 | MHATGLPISMKIFMYLLTVF | 3854 | 3855 |
| (Linker | LITQMIGSALFAVYLHRRLD | | |
| (MH); CD40L (aa | KIEDERNLHEDFVFMKTIQR | | |
| 14-261 of WT); | CNTGERSLSLLNCEEIKSQF | | |
| stop) | EGFVKDIMLNKEETKKENSF | | |
| | EMQKGDQNPQIAAHVISEAS | | |
| | SKTTSVLQWAEKGYYTMS | | |
| | NNLVTLENGKQLTVKRQGLY | | |
| | YIYAQVTFCSNREASSQAPF | | |
| | IASLCLKSPGRFERILLRAA | | |
| | NTHSSAKPCGQQSIHLGGVF | | |
| | ELQPGASVFVNVTDPSQVSH | | |
| | GTGFTSFGLLKL* | | |
| OT-001668 | MHMIETYNQTSPRSAATGLP | 3856 | 3857 |
| (Linker | ISMKIFMYLLTVFLITQMIG | | |
| (MH); CD40L | SALFAVYLHRRLDKIEDERN | | |
| (aa 1-261 of | LHEDFVFMKTIQRCNTGERS | | |
| WT, (S110- | LSLLNCEEIKSQFEGFVKDI | | |
| G116) del); | MLNKEETKKENDQNPQIAAH | | |
| stop) | VISEASSKTTSVLQWAEKGY | | |
| | YTMSNNLVTLENGKQLTVKR | | |
| | QGLYYIYAQVTFCSNREASS | | |
| | QAPFIASLCLKSPGRFERIL | | |
| | LRAANTHSSAKPCGQQSIHL | | |
| | GGVFELQPGASVFVNVTDPS | | |
| | QVSHGTGFTSFGLLKL* | | |
| OT-001663 | MEETRELQSLAAAVVPSAQT | 6402 | 6403 |
| (Met; | LKITDFSFSDFELSDLETAL | | |
| hPDE5 | CTIRMFTDLNLVQNFQMKHE | | |
| (aa 535-860 | VLCRWILSVKKNYRKNVAYH | | |
| of WT, H653A, | NWRHAFNTAQCMFAALKAGK | | |
| R732L); | IQNKLTDLEILALLIAALSA | | |
| MH Linker; | DLDHRGVNNSYIQRSEHPLA | | |
| CD40L; stop) | QLYCHSIMEHHHFDQCLMIL | | |
| | NSPGNQILSGLSIEEYKTTL | | |
| | KIIKQAILATDLALYIKRLG | | |
| | EFFELIRKNQFNLEDPHQKE | | |
| | LFLAMLMTACDLSAITKPWP | | |
| | IQQRIAELVATEFFDQGDRE | | |
| | RKELNIEPTDLMNREKKNKI | | |
| | PSMQVGFIDAICLQLYEALT | | |
| | HVSEDCFPLLDGCRKNRQKW | | |
| | QALAEQQMHMIETYNQTSPR | | |
| | SAATGLPISMKIFMYLLTVF | | |
| | LITQMIGSALFAVYLHRRLD | | |
| | KIEDERNLHEDFVFMKTIQR | | |
| | CNTGERSLSLLNCEEIKSQF | | |
| | EGFVKDIMLNKEETKKENSF | | |
| | EMQKGDQNPQIAAHVISEAS | | |
| | SKTTSVLQWAEKGYYTMSNN | | |
| | LVTLENGKQLTVKRQGLYYI | | |
| | YAQVTFCSNREASSQAPFIA | | |
| | SLCLRSPGRFERILLRAANT | | |
| | HSSARPCGQQSIHLGGVFEL | | |
| | QPGASVFVNVTDPSQVSHGT | | |
| | GFTSFGLLKL* | | |
| OT-001664 | MEETRELQSLAAAVVPSAQT | 6404 | 6405 |
| (Met; | LKITDFSFSDFELSDLETAL | | |
| hPDE5 | CTIRMFTDLNLVQNFQMKHE | | |
| (aa 535-860 | VLCRWILSVKKNYRKNVAYH | | |
| of WT, | NWRHAFNTAQCMFAALKAGK | | |
| R732L, | IQNKLTDLEILALLIAALSH | | |
| F736A); | DLDHRGVNNSYIQRSEHPLA | | |
| MH Linker; | QLYCHSIMEHHHFDQCLMIL | | |
| CD40L; stop) | NSPGNQILSGL | | |
| | SIEEYKTTLKIIKQAILATD | | |
| | LALYIKRLGEFAELIRKNQF | | |
| | NLEDPHQKELFLAMLMTACD | | |
| | LSAITKPWPIQQRIAELVAT | | |
| | EFFDQGDRERKELNIEPTDL | | |
| | MNREKKNKIPSMQVGFIDAI | | |
| | CLQLYEALTHVSEDCFPLLD | | |
| | GCRKNRQKWQALAEQQMHMI | | |
| | ETYNQTSPRSAATGLPISMK | | |
| | IFMYLLTVTLITQMIGSALF | | |
| | AVYLHRRLDKIEDERNLHED | | |
| | FVFMKTIQRCNTGERSLSLL | | |
| | NCEEIKSQFEGFVKDIMLNK | | |
| | EETKKENSFEMQKGDQNPQI | | |
| | AAHVISEASSKTTSVLQWAE | | |
| | KGYYTMSNNLVTLENGKQLT | | |
| | VKRQGLYYIYAQVTFCSNRE | | |
| | ASSQAPFIASLCLKSPGRFE | | |
| | RILLRAANTHSSAKPCGQQS | | |
| | IHLGGVFELQPGASVFVNVT | | |
| | DPSQVSHGTGFTSFGLLKL* | | |
| OT-001892 (Met; | MEETRELQSLAAAVVPSAQT | 6534 | 6535 |
| hPDE5 AA | LKITDFSFSDFELSDLETAL | | |
| 535-860 | CTIRMFTDLNLVQNFQMKHE | | |
| of WT (R732L); | VLCRWILSVKKNYRKNVAYH | | |
| Linker | NWRHAFNTAQCMFAALKAGK | | |
| (GGSGGG | IQNKLTDLEILALLIAALSH | | |
| SGGGSG); | DLDHRGVNNSYIQRSEHPLA | | |
| CD40L; stop) | QLYCHSIMEHHHFDQCLMIL | | |
| | NSPGNQILSGLSIEEYKTTL | | |
| | KIIKQAILATDLALYIKRLG | | |
| | EFFELIRKNQFNLEDPHQKE | | |
| | LFLAMLMTACDLSAITKPWT | | |
| | IQQRIAELVATEFFDQGDRE | | |
| | RKELNIEPTDLMNREKKNKI | | |
| | PSMQVGFIDAICLQLYEALT | | |
| | HVSEDCFPLLDGCRKNRQKW | | |
| | QALAEQQGGSGGGSGGGSGM | | |
| | IETYNQTSPRSAATGLPISM | | |
| | KIFMYLLTVFLITQMIGSAL | | |
| | FAVYLHRRLDKIEDERNLHE | | |
| | DFVFMKTIQRCNTGERSLSL | | |
| | LNCEEIKSQFEGFVKDIMLN | | |
| | KEETKKENSFEMQKGDQNPQ | | |
| | IAAHVISEASSKTTSVLQWA | | |
| | EKGYYTMSNNLVTLENGKQL | | |
| | TVKRQGLYYIYAQVTFCSNR | | |
| | EASSQAPFIASLCLKSPGRF | | |
| | ERILLRAANTHSSAKPCGQQ | | |
| | SIHLGGVFELQPGASVFVNV | | |
| | TDPSQVSHGTGFTSFGLLKL | | |
| | * | | |
| OT-001605 | MALPVTALLLPLALLLHAAR | 6539 | 6540 |
| (CD8a | PDIQMTQTTSSLSASLGDRV | | |
| leader; | TISCRASQDISKYLNWTQQK | | |
| CD19 scFv; | PDGWKLLIYHTSRLHSGVPS | | |
| CD8a Hinge | RFSGSGSGTDYSLTISNLEQ | | |
| and | EDIATYFCQQGNTLPYTFGG | | |
| Transmembrane | GTKLEITGGGGSGGGGSG | | |
| Domain; | GGGSEVKLQESGPGLVAPSQ | | |
| CD28 co- | SLSVTCTVSGVSLPDYGVSW | | |
| stimulatory | IRQPPRKGLEWLGVIWGSET | | |
| domain/4-1BB | TYYNSALKSRLTIIKDNSKS | | |
| intracellular | QVFLKMNSLQTDDTAIYYCA | | |
| domain; | KHYYYGGSYAMDYWGQGTSV | | |
| CD3 zea | TVSSTTTPAPRPPTPAPTIA | | |
| intracellular | SQPLSLRPEACRPAAGGAVH | | |
| domain; | TRGLDFACDIYIWAPLAGTC | | |
| Linker | GVLLLSLVITLYCKRGRKKL | | |
| (GS); P2A | LYIFKQPFMRPVQTTQEEDG | | |
| cleavage site; | CSCRFPEEEEGGCELRVKFS | | |
| Linker | RSADAPAYKQGQNQLYNELN | | |
| (GS); | LGRREEYDVLDKRRGRDPEM | | |
| Linker (MH); | GGKPRRKNPQEGLYNELQKD | | |
| CD40L; stop) | KMAEAYSEIGMKGERRRGKG | | |
| | HDGLYQGLSTATKDTYDALH | | |
| | MQALPPRGSATNFSLLKQAG | | |
| | DVEENPGPGSMHMIETYNQT | | |
| | SPRSAATGLPISMKIFMYLL | | |
| | TVFLITQMIGSALFAVYLHR | | |
| | RLDKIEDERNLHEDFVFMKT | | |
| | IQRCNTGERSLSLLNCEEIK | | |
| | SQFEGFVKDIMLNKEETKKE | | |
| | NSFEMQKGDQNPQIAAHVIS | | |
| | EASSKTTSVLQWAEKGYYTM | | |
| | SNNLVTLENGKQLTVKRQGL | | |
| | YYIYAQVTFCSNREASSQAP | | |
| | FIASLCLKSPGRFERILLRA | | |
| | ANTHSSAKPCGQQSIHLGGV | | |
| | FELQPGASVFVNVTDPSQVS | | |
| | HGTGFTSFGLLKL* | | |
| OT-001607 | — | — | 6541 |
| (Full | | | |
| Construct | | | |
| (CD8a | | | |
| leader; | | | |
| CD19 scFv; | | | |
| CD8a Hinge | | | |
| and | | | |
| Transmembrane | | | |
| Domain; | | | |
| CD28 co- | | | |
| stimulatory | | | |
| domain/4-1BB | | | |
| intracellular | | | |
| domain; | | | |
| CD3 zeta | | | |
| intracellular | | | |
| domain; | | | |
| stop; | | | |
| Spacer; IRES; | | | |
| Met; | | | |
| Linker (GS); | | | |
| His; | | | |
| CD40L; stop) | | | |
| OT-001607 | MALPVTALLLPLALLLHAAR | 6542 | 6543 |
| (Encoded | PDIQMTQTTSSLSASLGDRV | | |
| protein 1 | TISCRASQDISKYLNWYQQK | | |
| (CD8α | PDGTVKLLl | | |
| Leader; | YHTSRLHSGVPSRFSGSGSG | | |
| CD19 scFv; | TDYSLTISNLEQEDIATYFC | | |
| CD8a | QQGNTLPYTFGGGTKLEITG | | |
| Hinge and | GGGSGGGGSGGGGSEVKLQE | | |
| Transmembrane | SGPGLVAPSQSLSVTCTVSG | | |
| Domain; | VSLPDYGVSWIRQPPRKGLE | | |
| CD28 co- | WLGVIWGSETTYYNSALKSR | | |
| stimulatory | LTIIKDNSKSQVFLKMNSLQ | | |
| domain/4-1BB | TDDTAIYYCAKHYYYGGSYA | | |
| intracellular | MDYWGQGTSVTVSSTTTPAP | | |
| domain; | RPPTPAPTIASQPLSLRPEA | | |
| CD3 zeta | CRPAAGGAVHTRGLDFACDI | | |
| intracellular | YIWAPLAGTCGVLLLSLVIT | | |
| domain)) | LYCKRGRKKLLYIFKQPFMR | | |
| | PVQTTQEEDGCSCRFPEEEE | | |
| | GGCELRVKFSRSADAPAYKQ | | |
| | GQNQLYNELNLGRREEYDVL | | |
| | DKRRGRDPEMGGKPRRKNPQ | | |
| | EGLYNELQKDKMAEAYSEIG | | |
| | MKGERRRGKGHDGLYQGLST | | |
| | ATKDTYDALHMQALPPR | | |
| OT-001607 | MIETYNQTSPRSAATGLPIS | 6544 | 6545 |
| (Encoded | MKIFMYLLTVFLITQMIGSA | | |
| protein 2 | LFAVYLHRRLDKIEDERNLH | | |
| (CD40L; | EDFVFMKTIQRCNTGERSLS | | |
| stop)) | LLNCEEIKSQFEGFVKDIML | | |
| | NKEETKKENSFEMQKGDQNP | | |
| | QIAAHVISEASSKTTSVLQW | | |
| | AEKGYYTMSNNLVTLENGKQ | | |
| | LTVKRQGLYYIYAQVTFCSN | | |
| | REASSQAPFIASLCLKSPGR | | |
| | FERILLRAANTHSSAKPCGQ | | |
| | QSIHLGGVFELQPGASVFVN | | |
| | VTDPSQVSHGTGFTSFGLLK | | |
| | L* | | |
| OT-001966 | MSLALSLTADQMVSALLDAE | 6546 | 6547 |
| (Met; ER | PPILYSEYDPTRPFSEASMM | | |
| (aa 305-549 | GLLTNLADRELVHMINWAKR | | |
| of WT, | VPGFVDLTLHDQVHLLECAW | | |
| L384M, N413D, | MEILMIGLVWRSMEHPGKLL | | |
| M421G, | FAPNLLLDRDQGKCVEGGVE | | |
| G521R, | IFDMLLATSSRFRMMNLQGE | | |
| Y537S); | EFVCLKSIILLNSGVYTFLS | | |
| Linker | STLKSLEEKDHIHRVLDKIT | | |
| (GGSGGGS | DTLIHLMAKAGLTLQQQHQR | | |
| GGGSG); | LAQLLLILSHIRHMSNKRME | | |
| CD40L; stop) | HLYSMKCKNVVPLSDLLLEM | | |
| | LDAHRLGGSGGGSGGGSGMI | | |
| | ETYNQTSPRSAATGLPISMK | | |
| | IFMYLLTVFLITQMIGSALF | | |
| | AVYLHRRLDKIEDERNLHED | | |
| | FVFMKTIQRCNTGERSLSLL | | |
| | NCEEIKSQFEGFVKDIMLNK | | |
| | EETKKENSFEMQKGDQNPQI | | |
| | AAHVISEASSKTTSVLQWAE | | |
| | KGYYTMSNNLVTLENGKQLT | | |
| | VKRQGLYYIYAQVTFCSNRE | | |
| | ASSQAPFIASLCLKSPGRFE | | |
| | RILLRAANTHSSAKPCGQ | | |
| | QSIHLGGVFELQPGASVFVN | | |
| | VTDPSQVSHGTGFTSFGLLK | | |
| | L* | | |
| OT-001962 | MVGSLNCIVAVSQNMGIGKN | 6550 | 6551 |
| (hDHFR | GDLPWPPLRNEFRYFERMTT | | |
| (Q36E,Q103H, | TSSVEGKQNLVIMGKKTWFS | | |
| Y1221); | IPEKNRPLKGRINLVLSREL | | |
| Linker (H); | KEPPQGAHFLSRSLDDALKL | | |
| CD40L; stop) | TEHPELANKVDMVWIVGGSS | | |
| | VIKEAMNHPGHLKLFVTRIM | | |
| | QDFESDTFFPEIDLEKYKLL | | |
| | PEYPGVLSDVQEEKGIKYKF | | |
| | EVYEKNDHMIETYNQTSPRS | | |
| | AATGLPISMKIFMYLLTVFL | | |
| | ITQMIGSALFAVYLHRRLDK | | |
| | IEDERNLHEDFVFMKTIQRC | | |
| | NTGERSLSLLNCEEIKSQFE | | |
| | GFVKDIMLNKEETKKENSFE | | |
| | MQKGDQNPQIAAHVISEASS | | |
| | KTTSVLQWAEKGYYTMSNNL | | |
| | VTLENGKQLTVKRQGLYYIY | | |
| | AQVTFCSNREASSQAPFIAS | | |
| | LCLKSPGRFERILLRAANTH | | |
| | SSAKPCGQQSIHLGGVFELQ | | |
| | PGASVFVNVTDPSQVSHGTG | | |
| | FTSFGLLKL* | | |
| OT-002078 | MGGSGGGSGGGSGMIETYNQ | 6610 | 6611 |
| (Met; | TSPRSAATGLPISMKIFMYL | | |
| Linker | LTVFLITQMIGSALFAVYLH | | |
| ((GGSG)3); | RRLDKIEDERNLHEDFVFMK | | |
| CD40L (H224G, | TIQRCNTGERSLSLLNCEEI | | |
| G226F); stop) | KSQFEGFVKDIMLNKEETKK | | |
| | ENSFEMQKGDQNPQIAAHVI | | |
| | SEASSKTTSVLQWAEKGYYT | | |
| | MSNNLVTLENGKQLTVKRQG | | |
| | LYYIYAQVTFCSNREASSQA | | |
| | PFIASLCLKSPGRFERILLR | | |
| | AANTHSSAKPCGQQSIGLFG | | |
| | VFELQPGASVFVNVTDPSQV | | |
| | SHGTGFTSFGLLKL* | | |
| OT-002079 | MGGSGGGSGGGSGMIETYNQ | 6612 | 6613 |
| (Met; | TSPRSAATGLPISMKIFMYL | | |
| Linker | LTVFLITQMIGSALFAVYLH | | |
| ((GGSG)3); | RRLDKIEDERNLHEDFVFMK | | |
| CD40L (H224G, | TIQRCNTGERSLSLLNCEEI | | |
| G226H); | KSQFEGFVKDIMLNKEETKK | | |
| stop) | ENSFEMQKGDQNPQIAAHVI | | |
| | SEASSKTTSVLQWAEKGYYT | | |
| | MSNNLVTLENGKQLTVKRQG | | |
| | LYYIYAQVTFCSNREASSQA | | |
| | PFIASLCLKSPGRFERILLR | | |
| | AANTHSSAKPCGQQSIGLHG | | |
| | VFELQPGASVFVNVTDPSQV | | |
| | SHGTGFTSFGLLKL* | | |
| OT-002080 | MGGSGGGSGGGSGMIETYNQ | 6614 | 6615 |
| (Met; | TSPRSAATGLPISMKIFMYL | | |
| Linker | LTVFLITQMIGSALFAVYLH | | |
| ((GGSG)3); | RRLDKIEDERNLHEDFVFMK | | |
| CD40L | TIQRCNTGERSLSLLNCEEI | | |
| (Y172G, | KSQF | | |
| G226F); | EGFVKDIMLNKEETKKENSF | | |
| stop) | EMQKGDQNPQIAAHVISEAS | | |
| | SKTTSVLQWAEKGYYTMSNN | | |
| | LVTLENGKQLTVKRQGLYYI | | |
| | GAQVTFCSNREASSQAPFIA | | |
| | SLCLKSPGRFERILLRAANT | | |
| | HSSAKPCGQQSIHLFGVFEL | | |
| | QPGASVFVNVTDPSQVSHGT | | |
| | GFTSFGLLKL* | | |
| OT-002082 | MGGSGGGSGGGSGMIETYNQ | 6618 | 6619 |
| (Met; | TSPRSAATGLPISMKIFMYL | | |
| Linker | LTVFLITQMIGSALFAVYLH | | |
| ((GGSG)3); | RRLDKIEDERNLHEDFVFMK | | |
| CD40L | TIQRCNTGERSLSLLNCEEI | | |
| (H125G, | KSQFEGFVKDIMLNKEETKK | | |
| G227F); | ENSFEMQKGDQNPQIAAGVI | | |
| stop) | SEASSKTTSVLQWAEKGYYT | | |
| | MSNNLVTLENGKQLTVKRQG | | |
| | LYYIYAQVTFCSNREASSQA | | |
| | PFIASLCLKSPGRFERILL | | |
| | RAANTHSSAKPCGQQSIHLG | | |
| | FVFELQPGASVFVNVTDPSQ | | |
| | VSHGTGFTSFGLLKL* | | |
| OT-001967 | MSLALSLTADQMVSALLDAE | 6620 | 6621 |
| (Met; ER | PPILYSEYDPTRPFSEASMM | | |
| (aa 305-549 | GLLTNLADRELVHMINWAKR | | |
| of WT, | VPGFVDLTLHDQVHLLECAW | | |
| L384M, | MEILMIGLVWRSMEHPGKLL | | |
| N413D, | FAPNLLLDRDQGKCVEGGVE | | |
| M421G, | IFDMLLATSSRFRMMNLQGE | | |
| G521R, | EFVCLKSIILLNSGVYTFLS | | |
| Y537S); | STLKSLEEKDHIHRVLDKIT | | |
| linker (GS); | DTLIHLMAKAGLTLQQQHQR | | |
| CD40L; stop) | LAQLLLILSHIRHMSNKRME | | |
| | HLYSMKCKNVVPLSDLLLEM | | |
| | LDAHRLGSMIETYNQTSPRS | | |
| | AATGLPISMKIFMYLLTVFL | | |
| | ITQMIGSALFAVYLHRRLDK | | |
| | IEDERNLHEDFVFMKTIQRC | | |
| | NTGERSLSLLNCEEIKSQFE | | |
| | GFVKDIMLNKEETKKENSFE | | |
| | MQKGDQNPQIAAHVISEASS | | |
| | KTTSVLQWAEKGYYTMSNNL | | |
| | VTLENGKQLTVKRQGLYYIY | | |
| | AQVTFCSNREASSQAPFIAS | | |
| | LCLKSPGRFERILLRAANTH | | |
| | SSAKPCGQQSIHLGGVFELQ | | |
| | PGASVFVNVTDPSQVSHGTG | | |
| | FTSFGLLKL* | | |
| OT-001965 | MSLALSLTADQMVSALLDAE | 6622 | 6623 |
| (Met; ER | PPILYSEYDPTRPFSEASMM | | |
| (aa 305-549 | GLLTNLADRELVHMINWAKR | | |
| of WT, | VPGFVDLTLHDQVHLLECAW | | |
| L384M, | MEILMIGLVWRSMEHPGKLL | | |
| N413D, | FAPNLLLDRDQGKCVEGGVE | | |
| M421G, | IFDMLLATSSRFRMMNLQGE | | |
| G521R, | EFVCLKSIILLNSGVYTFLS | | |
| Y537S); | STLKSLEEKDHIHRVLDKIT | | |
| linker | DTLIHLMAKAGLTLQQQHQR | | |
| (GSG); | LAQLLLILSHIRHMSNKRME | | |
| CD40L; stop) | HLYSMKCKNVVPLSDLLLEM | | |
| | LDAHRLGSGMIETYNQTSPR | | |
| | SAATGLPISMKIFMYLLTVF | | |
| | LITQMIGSALFAVYLHRRLD | | |
| | KIEDERNLHEDFVFMKTIQR | | |
| | CNTGERSLSLLNCEEIKSQF | | |
| | EGFVKDIMLNKEETKKENSF | | |
| | EMQKGDQNPQIAAHVISEAS | | |
| | SKTTSVLQWAEKGYYTMSNN | | |
| | LVTLENGKQLTVKRQGLYYI | | |
| | YAQVTFCSNREASSQAPFIA | | |
| | SLCLKSPGRFERILLRAANT | | |
| | HSSAKPCGQQSIHLGGVFEL | | |
| | QPGASVFVNVTDPSQVSHGT | | |
| | GFTSFGLLKL* | | |
| OT-001961 | MVGSLNCIVAVSQNMGIGKN | 6624 | 6625 |
| (Met; | GDLPWPPLRNEFRYFQRMTT | | |
| hDHFR | TSSVEGKQNLVIMGKKTWFS | | |
| (2-187 of | IPEKNRPLKGRINLVLSREL | | |
| WT, Y1221); | KEPPQGAHFLSRSLDDALKL | | |
| Linker | TEQPELANKVDMVWIVGGSS | | |
| (H); CD40L; | VIKEAMNHPGHLKLFVTRIM | | |
| stop) | QDFESDTFFPEIDLEKYKLL | | |
| | PEYPGVLSDVQEEKGIKYKF | | |
| | EVYEKNDHMIETYNQTSPRS | | |
| | AATGLPISMKIFMYLLTVFL | | |
| | ITQMIGSALFAVYLHRRLDK | | |
| | IEDERNLHEDFVFMKTIQRC | | |
| | NTGERSLSLLNCEEIKSQFE | | |
| | GFVKDIMLNKEETKKENSFE | | |
| | MQKGDQNPQIAAHVISEASS | | |
| | KTTSVLQWAEKGYYTMSNNL | | |
| | VTLENGKQLTVKRQGLYYIY | | |
| | AQVTFCSNREASSQAPFIAS | | |
| | LCLKSPGRFERILLRAANTH | | |
| | SSAKPCGQQSIHLGGVFELQ | | |
| | PGASVFVNVTDPSQVSHGTG | | |
| | FTSFGLLKL* | | |
| OT-001963 | MVGSLNCIVAVSQNMGIGKN | 6626 | 6627 |
| (Met; | GDLPWPPLRNEFRYFQRMTT | | |
| hDHFR | TSSVEGKQNLVIMGRKTWFS | | |
| (2-187 of | IPEKKRPLKGRINLVLSREL | | |
| WT, | KEPPQGAHFLSRSLDDALKL | | |
| K55R, N65K, | TEQPELANKVDMVWIVGGSS | | |
| Y122I); | VIKEAMNHPGHLKLFVTRIM | | |
| Linker (H); | QDFESDTFFPEIDLEKYKLL | | |
| CD40L; | PEYPGVLSDVQEEKGIKYKF | | |
| stop) | EVYEKNDHMIETYNQTSPRS | | |
| | AATGLPISMKIFMYLLTVFL | | |
| | ITQMIGSALFAVYLHRRLDK | | |
| | IEDERNLHEDFVFMKTIQRC | | |
| | NTGERSLSLLNCEEIKSQFE | | |
| | GFVKDIMLNKEETKKENSFE | | |
| | MQKGDQNPQIAAHVISEASS | | |
| | KTTSVLQWAEKGYYTMSNNL | | |
| | VTLENGKQLTVKRQGLYYIY | | |
| | AQVTFCSNREASSQAPFIAS | | |
| | LCLKSPGRFERILLRAANTH | | |
| | SSAKPCGQQSIHLGGVFELQ | | |
| | PGASVFVNVTDPSQVSHGTG | | |
| | FTSFGLLKL* | | |
| OT-001671 | MSGISLIAALAVDYVIGMEN | 6628 | 6629 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (aa 2-159 of | DEAIAACGDVPEIMVIGGGR | | |
| WT, R12Y, | VIEQFLPKAQKLYLTHIDAE | | |
| Y100I); | VEGDTHFPDYEPDDWESVFS | | |
| Linker (H); | EFHDADAQNSHSYCFEILER | | |
| CD40L | RHMIETYNQTSPRSAATGLP | | |
| (aa 1-261 of | ISMKIFMYLLTVFLITQMIG | | |
| WT, | SALFAVYLHRRLDKIEDERN | | |
| (S110-G116) | LHEDFVFMKTIQRCNTGERS | | |
| del); | LSLLNCEEIKSQFEGFVKDI | | |
| stop) | MLNKEETKKENDQNPQIAAH | | |
| | VISEASSKTTSVLQWAEKGY | | |
| | YTMSNNLVTLENGKQLTVKR | | |
| | QGLYYIYAQVTFCSNREASS | | |
| | QAPFIASLCLKSPGRFERIL | | |
| | LRAANTHSSAKPCGQQSIHL | | |
| | GGVFELQPGASVFVNVTDPS | | |
| | QVSHGTGFTSFGLLKL* | | |
| OT-002106 | MSLALSLTADQMVSALLDAE | 6630 | 6631 |
| (Met; ER | PPILYSEYDPTRPFSEASMM | | |
| (aa 305-549 | GLLTNLADRELVHMINWAKR | | |
| of WT, | VPGFVDLTLHDQVHLLECAW | | |
| L384M, | MEILMIGLVWRSMEHPGKLL | | |
| N413D, | FAPNLLLDRDQGKCVEGGVE | | |
| M421G, | IFDMLLATSSRFRMMNLQGE | | |
| G521R, | EFVCLKSIILLNSGVYTFLS | | |
| Y537S); | STLKSLEEKDHIHRVLDKIT | | |
| CD8 | DTLIHLMAKAGLTLQQQHQR | | |
| cytoplasmic | LAQLLLILSHIRHMSNKRME | | |
| tail; | HLYSMKCKNVVPLSDLLLEM | | |
| Linker; (GS); | LDAHRLNHRNRRGSMIETYN | | |
| CD40L; stop | QTSPRSAATGLPISMKIFMY | | |
| | LLTVFLITQMIGSALFAVYL | | |
| | HRRLDKIEDERNLHEDFVFM | | |
| | KTIQRCNTGERSLSLLNCEE | | |
| | IKSQFEGFVKDIMLNKEETK | | |
| | KENSFEMQKGDQNPQIAAHV | | |
| | ISEASSKTTSVLQWAEKGYY | | |
| | TMSNNLVTLENGKQLTVKRQ | | |
| | GLYYIYAQVTFCSNREASSQ | | |
| | APFIASLCLKSPGRFERILL | | |
| | RAANTHSSAKPCGQQSIHLG | | |
| | GVFELQPGASVFVNVTDPSQ | | |
| | VSHGTGFTSFGLLKL* | | |
| OT-002107 | MNHRNRRGSSLALSLTADQM | 6632 | 6633 |
| (Met; | VSALLDAEPPILYSEYDPTR | | |
| CD8 | PFSEASMMGLLTNLADRELV | | |
| cytoplasmic | HMINWAKRVPGFVDLTLHDQ | | |
| tail); | VHLLECAWMEILMIGLVWRS | | |
| Linker; | MEHPGKLLFAPNLLLDRDQG | | |
| (GS); | KCVEGGVEIFDMLLATSSRF | | |
| ER | RMMNLQGEEFVCLKSIILLN | | |
| (aa 305-549 | SGVYTFLSSTLKSLEEKDHI | | |
| of | HRVLDKITDTLIHLMAKAGL | | |
| WT, L384M, | TLQQQHQRLAQLLLILSHIR | | |
| N413D, | HMSNKRMEHLYSMKCKNVVP | | |
| M421G, | LSDLLLEMLDAHRLGGSGGG | | |
| G521R, | SGGGSGMIETYNQTSPRSAA | | |
| Y537S); | TGLPISMKIFMYLLTVFLIT | | |
| Linker | QMIGSALFAVYLHRRLDKIE | | |
| ((GGSG)3); | DERNLHEDFVFMKTIQRCNT | | |
| CD40L; | GERSLSLLNCEEIKSQFEGF | | |
| stop) | VKDIMLNKEETKKENSFEMQ | | |
| | KGDQNPQIAAHVISEASSKT | | |
| | TSVLQWAEKGYYTMSNNLVT | | |
| | LENGKQLTVKRQGLYYIYAQ | | |
| | VTFCSNREASSQAPFIASLC | | |
| | LKSPGRFERILLRAANTHSS | | |
| | AKPCGQQSIHLGGVFELQPG | | |
| | ASVFVNVTDPSQVSHGTGFT | | |
| | SFGLLKL* | | |
| OT-002021 | MSGISLIAALAVDRVIGMEN | 6634 | 6635 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100A); | VAEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002022 | MSGISLIAALAVDRVIGMEN | 6636 | 6637 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100C); | VCEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002023 | MSGISLIAALAVDRVIGMEN | 6638 | 6639 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100D); | VDEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002024 | MSGISLIAALAVDRVIGMEN | 6640 | 6641 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100E); | VEEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002025 | MSGISLIAALAVDRVIGMEN | 6642 | 6643 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100F); | VFEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002026 | MSGISLIAALAVDRVIGMEN | 6644 | 6645 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100G); | VGEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002027 | MSGISLIAALAVDRVIGMEN | 6646 | 6647 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100H); | VHEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002028 | MSGISLIAALAVDRVIGMEN | 6648 | 6649 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y1001); | VIEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002029 | MSGISLIAALAVDRVIGMEN | 6650 | 6651 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100K); | VKEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFLASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002030 | MSGISLIAALAVDRVIGMEN | 6652 | 6653 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100L); | VLEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002031 | MSGISLIAALAVDRVIGMEN | 6654 | 6655 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100M); | VMEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002032 | MSGISLIAALAVDRVIGMEN | 6656 | 6657 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100N); | VNEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002033 | MSGISLIAALAVDRVIGMEN | 6658 | 6659 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100P); | VPEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002034 | MSGISLIAALAVDRVIGMEN | 6660 | 6661 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100Q); | VQEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002035 | MSGISLIAALAVDRVIGMEN | 6662 | 6663 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100R); | VREQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002036 | MSGISLIAALAVDRVIGMEN | 6664 | 6665 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100S); | VGEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002037 | MSGISLIAALAVDRVIGMEN | 6666 | 6667 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100T); | VTEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002038 | MSGISLIAALAVDRVIGMEN | 6668 | 6669 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100V); | VVEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002039 | MSGISLIAALAVDRVIGMEN | 6670 | 6671 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT, Y100W); | VWEQFLPKAQKLYLTHIDAE | | |
| Linker | VEGDTHFPDYEPDDWESVFS | | |
| (H); CD40L; | EFHDADAQNSHSYCFEILER | | |
| stop) | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |
| OT-002040 | MSGISLIAALAVDRVIGMEN | 6672 | 6673 |
| (Met; | AMPWNLPADLAWFKRNTLNK | | |
| Linker (SG); | PVIMGRHTWESIGRPLPGRK | | |
| ecDHFR | NIILSSQPGTDDRVTWVKSV | | |
| (a 2-159 of | DEAIAACGDVPEIMVIGGGR | | |
| WT); | VYEQFLPKAQKLYLTHIDAE | | |
| Linker (H); | VEGDTHFPDYEPDDWESVFS | | |
| CD40L; stop) | EFHDADAQNSHSYCFEILER | | |
| | RHMIETYNQTSPRSAATGLP | | |
| | ISMKIFMYLLTVFLITQMIG | | |
| | SALFAVYLHRRLDKIEDERN | | |
| | LHEDFVFMKTIQRCNTGERS | | |
| | LSLLNCEEIKSQFEGFVKDI | | |
| | MLNKEETKKENSFEMQKGDQ | | |
| | NPQIAAHVISEASSKTTSVL | | |
| | QWAEKGYYTMSNNLVTLENG | | |
| | KQLTVKRQGLYYIYAQVTFC | | |
| | SNREASSQAPFIASLCLKSP | | |
| | GRFERILLRAANTHSSAKPC | | |
| | GQQSIHLGGVFELQPGASVF | | |
| | VNVTDPSQVSHGTGFTSFGL | | |
| | LKL* | | |

### B. Pharmaceutical Compositions and Formulations

The present teachings further comprise pharmaceutical compositions comprising one or more of the tunable protein expression systems, nucleic acids, polynucleotides, modified cells or payloads of the present disclosure, and optionally at least one pharmaceutically acceptable excipient or inert ingredient.

As used herein the term “pharmaceutical composition” refers to a preparation of one or more of the tunable protein expression systems, nucleic acids, polynucleotides, payloads or components described herein, or pharmaceutically acceptable salts thereof, optionally with other chemical components such as physiologically suitable carriers and excipients.

The term “excipient” or “inactive ingredient” refers to an inert or inactive substance added to a pharmaceutical composition to further facilitate administration of a compound.

In some embodiments, compositions are administered to humans, human patients or subjects. For the purposes of the present disclosure, the phrase “active ingredient” generally refers to any one or more tunable protein expression system components to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient or inert ingredient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. A healthcare practitioner skilled in the art may monitor efficacy of treatment or prevention by measuring any one of such parameters, or any combination of parameters. In connection with the administration of compositions of the present disclosure, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present disclosure can also be judged using an experimental animal model for the given disease as known in the art. When using an experimental animal model, efficacy of treatment is evidenced when a statistically significant change is observed.

1. Formulations

The compositions for example, polypeptides, proteins, polynucleotide and vector compositions of the present disclosure may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, the polynucleotide and vector formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the disclosure, the formulation may be selected from any of those taught, for example, in International Application PCT/US2012/069610.

2. Inactive Ingredients

In some embodiments, pharmaceutical or other formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present disclosure may be approved by the US Food and Drug Administration (FDA). Suitable inactive ingredients for formulations of the present disclosure can be found in Applicant's PCT International Publication Nos. WO2018/161000; WO2018/231759; WO2019/241315; and WO2018/237323.

3. Dosing, Delivery and Administration

The compositions of the disclosure may be delivered to a cell or a subject through one or more routes and modalities. The viral vectors containing one or more tunable protein expression systems, nucleic acids, polynucleotides, payloads, and other components described herein may be used to deliver them to a cell and/or a subject. Other modalities may also be used such as mRNAs, plasmids, and as recombinant proteins.

4. Naked Delivery

Pharmaceutical compositions, tunable protein expression systems, nucleic acids, polynucleotides, or payloads of the present disclosure may be delivered to cells, tissues, organs and/or organisms in naked form. As used herein in, the term “naked” refers to pharmaceutical compositions, tunable protein expression systems, nucleic acids, polynucleotides, or payloads delivered free from agents or modifications which promote transfection or permeability. The naked pharmaceutical compositions, tunable protein expression systems, nucleic acids, polynucleotides, or payloads may be delivered to the cells, tissues, organs and/or organisms using routes of administration known in the art and described herein. In some embodiments, naked delivery may include formulation in a simple buffer such as saline or PBS.

5. Formulated Delivery

In some embodiments, pharmaceutical compositions, tunable protein expression systems, nucleic acids, polynucleotides, or payloads of the present disclosure may be formulated, using methods described herein. Formulations may comprise pharmaceutical compositions, tunable protein expression systems, nucleic acids, polynucleotides, or payloads which may be modified and/or unmodified. Formulations may further include, but are not limited to, cell penetration agents, pharmaceutically acceptable carriers, delivery agents, bioerodible or biocompatible polymers, solvents, and/or sustained-release delivery depots. Formulations of the present disclosure may be delivered to cells using routes of administration known in the art and described herein.

Pharmaceutical compositions, tunable protein expression systems, nucleic acids, polynucleotides, or payloads may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

6. Delivery to Cells

In another aspect of the disclosure, polynucleotides encoding a tunable protein expression system, DRD, or payload of interest and compositions of the disclosure and vectors comprising said polynucleotides may be introduced into cells such as immune effector cells.

In one aspect of the disclosure, polynucleotides encoding a tunable protein expression system, DRD, or payload of interest and compositions of the disclosure, may be packaged into plasmids, viral vectors or integrated into viral genomes allowing transient or stable expression of the polynucleotides. Preferable viral vectors are retroviral vectors including lentiviral vectors and gamma retroviral vectors. In order to construct a retroviral vector, a polynucleotide molecule encoding a tunable protein expression system, DRD, or payload of interest is inserted into the viral genome in the place of certain viral sequences to produce a virus that is replication-defective. The recombinant viral vector is then introduced into a packaging cell line containing the gag, pol, and env genes, but without the LTR and packaging components. The recombinant retroviral particles are secreted into the culture media, then collected, optionally concentrated, and used for gene transfer. Lentiviral vectors are especially preferred as they are capable of infecting both dividing and non-dividing cells.

Vectors may also be transferred to cells by non-viral methods by physical methods such as needles, electroporation, sonoporation, hydroporation; chemical carriers such as inorganic particles (e.g. calcium phosphate, silica, gold) and/or chemical methods. In some embodiments, synthetic or natural biodegradable agents may be used for delivery such as cationic lipids, lipid nano emulsions, nanoparticles, peptide based vectors, or polymer based vectors. In some embodiments, vectors may be transferred to cells by temporary membrane disruption, for example, by high speed cell deformation.

In some embodiments, the polypeptides of the disclosure may be delivered to the cell directly. In one embodiment, the polypeptides of the disclosure may be delivered using synthetic peptides comprising an endosomal leakage domain (ELD) fused to a cell penetration domain (CLD). The polypeptides of the disclosure are co introduced into the cell with the ELD-CLD-synthetic peptide. ELDs facilitate the escape of proteins that are trapped in the endosome, into the cytosol. Such domains are derived proteins of microbial and viral origin and have been described in the art. CPDs allow the transport of proteins across the plasma membrane and have also been described in the art. The ELD-CLD fusion proteins synergistically increase the transduction efficiency when compared to the co-transduction with either domain alone. In some embodiments, a histidine rich domain may optionally be added to the shuttle construct as an additional method of allowing the escape of the cargo from the endosome into the cytosol. The shuttle may also include a cysteine residue at the N or C terminus to generate multimers of the fusion peptide. Multimers of the ELD-CLD fusion peptides generated by the addition of cysteine residue to the terminus of the peptide show even greater transduction efficiency when compared to the single fusion peptide constructs. The polypeptides of the disclosure may also be appended to appropriate localization signals to direct the cargo to the appropriate sub-cellular location e.g. nucleus. In some embodiments any of the ELDs, CLDs or the fusion ELD-CLD synthetic peptides taught in the International Patent Publication, WO2016161516 and WO2017175072 may be useful in the present disclosure (the contents of each of which are herein incorporated by reference in their entirety).

7. Delivery Modalities and/or Vectors

The tunable protein expression systems, DRDs, or payloads of interest of the present disclosure may be delivered using one or more modalities. The present disclosure also provides vectors that package polynucleotides of the disclosure encoding tunable protein expression systems, DRDs, or payload constructs, and combinations thereof. Vectors of the present disclosure may also be used to deliver the packaged polynucleotides to a cell, a local tissue site or a subject. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to an adenovirus, adeno-associated virus (AAV), alphavirus, flavivirus, herpes virus, measles virus, rhabdovirus, retrovirus, lentivirus, Newcastle disease virus (NDV), poxvirus, and picornavirus. In some embodiments, the virus is selected from a lentivirus vector, a gamma retrovirus vector, adeno-associated virus (AAV) vector, adenovirus vector, and a herpes virus vector.

In general, vectors contain an origin of replication functional in at least one organism, a promoter sequence and convenient restriction endonuclease site, and one or more selectable markers e.g. a drug resistance gene.

In some embodiments, the recombinant expression vector may comprise regulatory sequences, such as transcription and translation initiation and termination codons, which are specific to the type of host cell into which the vector is to be introduced.

In some embodiments, the vector of the disclosure may comprise one or more payloads taught herein, wherein the two or more payloads may be included in one ligand response. In this case, the two or more payloads are tuned by the same ligand or responsive agent simultaneously.

8. Lentiviral Vehicles/Particles

In some embodiments, lentiviral vehicles/particles may be used as delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope. Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. As used herein, the term “recombinant” refers to a vector or other nucleic acid containing both lentiviral sequences and non-lentiviral retroviral sequences.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the tunable protein expression system, DRD, and payload of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), FreeStyle™ 293 Expression System (ThermoFisher, Waltham, Mass.), and other HEK293T-based producer cell lines (e.g., Stewart et al., Hum Gene Ther. 2011, 22(3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelope proteins from other viruses, such as the G protein of vesicular stomatitis virus (VSV G) or baculoviral gp64 envelope proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof.

Methods for generating recombinant lentiviral particles are discussed in the art, for example, U.S. Pat. Nos. 8,846,385; 7,745,179; 7,629,153; 7,575,924; 7,179,903; and 6, 808, 905.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII.

9. Adeno-Associated Viral Particles

Delivery of polynucleotides of any of the tunable protein expression systems, DRDs, or payload constructs of the present disclosure may be achieved using recombinant adeno-associated viral (rAAV) vectors. Such vectors or viral particles may be designed to utilize any of the known serotype capsids or combinations of serotype capsids.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The tunable protein expression systems, DRDs, or payload constructs may be encoded in one or more viral genomes to be packaged in the AAV capsids taught herein.

Such vector or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

The tunable protein expression systems, DRDs, or payload constructs of the disclosure may be administered in one or more or separate AAV particles.

In some embodiments, the tunable protein expression systems may be administered in one or more AAV particles. In some embodiments, more than one tunable protein expression system, DRD or payload may be encoded in a viral genome.

10. Retroviral Vehicles/Particles (γ-Retroviral Vectors)

In some embodiments, retroviral vehicles/particles may be used to deliver the tunable protein expression systems, DRDs, or payload constructs of the present disclosure. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xprl) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present disclosure that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism.

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose of the disclosure.

In some embodiments, polynucleotides encoding the tunable protein expression systems, DRDs, or payload constructs are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

11. Oncolytic Viral Vector

In some embodiments, polynucleotides of present disclosure may be packaged into oncolytic viruses. As used herein, the term “oncolytic virus” refers to a virus that preferentially infects and kills cancer cells such as vaccine viruses. An oncolytic virus can occur naturally or can be a genetically modified virus such as oncolytic adenovirus, and oncolytic herpes virus.

In some embodiments, oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor; See e.g., U.S. Pat. No. 9,226,977.

12. Messenger RNA (mRNA)

In some embodiments, the tunable protein expression systems, DRD, or payloads of the disclosure may be designed as a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Application number PCT/US2013/030062.

In some embodiments, the effector modules may be designed as self-amplifying RNA. “Self-amplifying RNA” as used herein refers to RNA molecules that can replicate in the host resulting in the increase in the amount of the RNA and the protein encoded by the RNA. Such self-amplifying RNA may have structural features or components of any of those taught in International Patent Application Publication No. WO2011005799.

13. Dosing

The present disclosure provides methods comprising administering any one or more or component or composition of a tunable protein expression system to a subject in need thereof. These may be administered to a subject using any amount and any route of administration effective for preventing or treating or imaging a disease, disorder, and/or condition (e.g., a disease, disorder, and/or condition relating to cancer or an autoimmune disease). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions of the disclosure may be used for cancer immunotherapy in varying doses to avoid T cell exhaustion, prevent cytokine release syndrome and minimize toxicity associated with immunotherapy. For example, low doses of the compositions of the present disclosure may be used to initially treat patients with high tumor burden, while patients with low tumor burden may be treated with high and repeated doses of the compositions of the disclosure to ensure recognition of a minimal tumor antigen load. In another instance, the compositions of the present disclosure may be delivered in a pulsatile fashion to reduce tonic T cell signaling and enhance persistence in vivo. In some aspects, toxicity may be minimized by initially using low doses of the compositions of the disclosure, prior to administering high doses. Dosing may be modified if serum markers such as ferritin, serum C-reactive protein, IL6, IFN-γ, and TNF-α are elevated.

In some embodiments, the neurotoxicity may be associated with CAR or TIL therapy. Such neurotoxicity may be associated CD19-CARs. Toxicity may be due to excessive T cell infiltration into the brain. In some embodiments, neurotoxicity may be alleviated by preventing the passage of T cells through the blood brain barrier. This can be achieved by the targeted gene deletion of the endogenous alpha-4 integrin inhibitors such as tysabri/natalizumab may also be useful in the present disclosure.

Also provided herein are methods of administering ligands or DRD ligands in accordance with the disclosure to a subject in need thereof. The ligand may be administered to a subject or to cells, using any amount and any route of administration effective for tuning the tunable protein expression system, DRD, or payloads of the disclosure. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. In certain embodiments, the ligands in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 10 mg/kg to about 100 mg/kg, from about 50 mg/kg to about 500 mg/kg, from about 100 mg/kg to about 1000 mg/kg, of subject body weight per day, one or more times a day, to obtain the desired effect. In some embodiments, the dosage levels may be 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg or mg/kg of subject body weight per day, or more times a day, to obtain the desired effect.

The present disclosure provides methods for delivering to a cell or tissue any of the ligands described herein, comprising contacting the cell or tissue with said ligand and can be accomplished in vitro, ex vivo, or in vivo. In certain embodiments, the ligands in accordance with the present disclosure may be administered to cells at dosage levels sufficient to deliver from about 1 nM to about 10 nM, from about 5 nM to about 50 nM, from about 10 nM to about 100 nM, from about 50 nM to about 500 nM, from about 100 nM to about 1000 nM, from about 1 μM to about 10 μM from about 5 μM to about 50 μM from about 10 μM to about 100 μM from about 25 μM to about 250 μM from about 50 μM to about 500 μM. In some embodiments, the ligand may be administered to cells at doses selected from but not limited to 0.00064 μM, 0.0032 μM, 0.016 μM, 0.08 μM, 0.4 μM, 1 μM 2 μM, 10 μM, 50 μM, 75, μM, 100 μM, 150 μM, 175 μM, 200 μM, 250 μM.

The desired dosage of the ligands of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event. The desired dosage of the ligand of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

14. Administration

In some embodiments, the compositions for cancer immunotherapy or treatment of autoimmune disease may be administered to cells ex vivo and subsequently administered to the subject. Immune cells can be isolated and expanded ex vivo using a variety of methods known in the art. For example, methods of isolating cytotoxic T cells are described in U.S. Pat. Nos. 6,805,861 and 6,531,451. Isolation of NK cells is described in U.S. Pat. No. 7,435,596.

In some embodiments, depending upon the nature of the cells, the cells may be introduced into a host organism e.g. a mammal, in a wide variety of ways including by injection, transfusion, infusion, local instillation or implantation. In some aspects, the cells of the disclosure may be introduced at the site of the tumor. The number of cells that are employed will depend upon a number of circumstances, the purpose for the introduction, the lifetime of the cells, the protocol to be used, for example, the number of administrations, the ability of the cells to multiply, or the like. The cells may be in a physiologically-acceptable medium.

In some embodiments, the cells of the disclosure may be administered in multiple doses to subjects having a disease or condition. The administrations generally effect an improvement in one or more symptoms of cancer or a clinical condition and/or treat or prevent cancer or clinical condition or symptom thereof.

15. Routes of Delivery

The pharmaceutical compositions, tunable protein expression systems, nucleic acids, polynucleotides, payloads, vectors and cells of the present disclosure may be administered by any route to achieve a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

16. Parenteral and Injectable Administration

In some embodiments, pharmaceutical compositions, tunable protein expression systems, nucleic acids, polynucleotides, payloads, vectors and cells of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

17. Detectable Agents and Labels

The tunable protein expression systems, nucleic acids, polynucleotides, payloads, vectors and cells of the present disclosure may be associated with or bound to one or more radioactive agents or detectable agents.

These agents include various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., 18F, 67Ga, 81mKr, 82Rb, 111In, 123I, 133Xe, 201T1, 125I, 35S, 14C, 3H, or 99mTc (e.g., as pertechnetate (technetate(VII), TcO4-)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultra small superparamagnetic iron oxide (USPIO)), manganese chelates (e.g., Mn-DPDP), barium sulfate, iodinated contrast media (iohexol), microbubbles, or perfluorocarbons).

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

18. Applications and Uses

The tunable protein expression systems, constructs, ligands, or compositions of the present disclosure may be utilized in a large variety of applications including, but not limited to, therapeutics, diagnosis and prognosis, bioengineering, bioprocessing, biomanufacturing, research agents, metabolomics, gene expression, enzyme replacement, etc.

The present disclosure provides methods comprising administering a composition, for example, a pharmaceutical composition comprising one or more components of a tunable protein expression system to a subject in need thereof.

While there may be several uses that do not involve a medical treatment, for example, to generate cell lines and reagents for scientific research, one use involves the administration of the compositions of the present disclosure to generate in vivo gene therapy or modified cells for adoptive cell therapy, for example, the treatment of cancer, autoimmune diseases and other diseases. In an illustrative method of medical treatment or prevention of a disease, condition or disorder in a subject in need thereof, can include the following steps: (a) providing a population of cells (either human, animal, primary or cell culture, including autologous, allogenic or syngeneic); (b) introducing at least one nucleic acid molecule into at least one cell in the population of cells, wherein the at least one nucleic acid molecule comprises: (i) a first polynucleotide comprising a first nucleic acid sequence that encodes a protein of interest that treats the disease; a second nucleic acid sequence that encodes a drug responsive domain (DRD), wherein the payload nucleic acid sequence is operably linked to the DRD nucleic acid sequence that encodes a protein of interest that treats the disease; (c) delivering the cell into the subject; and (d) administering a ligand to the subject that stabilizes the DRD sufficiently to enable expression of the protein of interest in the cell; wherein expression of the protein of interest is regulated by the presence of ligand in the subject, and the amount and/or duration of ligand administration is sufficient to produce a therapeutically effective amount of the protein of interest, to treat the disease.

In the above method, the protein of interest can be used to ameliorate, cure, prevent or reduce one or more symptoms of the disease, condition or disorder.

The compositions of the present disclosure may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Also provided herein, are methods of administering one or more stabilizing ligands (as used herein, the ligand that stabilizes the DRD, may be called a stabilizing ligand or simply a ligand, with the understanding that the ligand is effective in stabilizing the DRD used in the tunable protein expression systems in accordance with the disclosure) to a subject in need thereof. The ligand may be administered to a subject or to cells, using any amount and any route of administration effective for tuning the amount of the protein of interest of the present disclosure in a cell transformed with the tunable protein expression system. The exact amount of stabilizing ligand required will vary from subject to subject, depending on the species, age, and general condition of the subject, the severity of the disease, the particular composition, its mode of administration, its mode of activity, and the like. The subject may be a human, a mammal, or an animal.

### C. Therapeutic Uses

1. Cancer Immunotherapy

Cancer immunotherapy aims at the induction or restoration of the reactivity of the immune system towards cancer. Significant advances in immunotherapy research have led to the development of various strategies which may broadly be classified into active immunotherapy and passive immunotherapy. In general, these strategies may be utilized to directly kill cancer cells or to counter the immunosuppressive tumor microenvironment. Active immunotherapy aims at induction of an endogenous, long-lasting tumor-antigen specific immune response. The response can further be enhanced by non-specific stimulation of immune response modifiers such as cytokines. In contrast, passive immunotherapy includes approaches where effector immune molecules such as tumor-antigen specific cytotoxic T cells or antibodies are administered to the host. This approach is short lived and requires multiple applications.

Despite significant advances, the efficacy of current immunotherapy strategies is limited by associated toxicities. These are often related to the narrow therapeutic window associated with immunotherapy, which in part, emerges from the need to push therapy dose to the edge of potentially fatal toxicity to get a clinically meaningful treatment effect. Further, dose expands in vivo since adoptively transferred immune cells continue to proliferate within the patient, often unpredictably.

A major risk involved in immunotherapy is the on-target but off tumor side effects resulting from T-cell activation in response to normal tissue expression of the tumor associated antigen (TAA). Clinical trials utilizing T cells expressing T-cell receptor against specific TAA reported skin rash, colitis and hearing loss in response to immunotherapy.

Immunotherapy may also produce on target, on-tumor toxicities that emerge when tumor cells are killed in response to the immunotherapy. The adverse effects include tumor lysis syndrome, cytokine release syndrome and the related macrophage activation syndrome. Importantly, these adverse effects may occur during the destruction of tumors, and thus even a successful on-tumor immunotherapy might result in toxicity. Approaches to control immunotherapy via immunotherapeutic agent regulation are thus highly desirable since they have the potential to reduce toxicity and maximize efficacy.

The present disclosure provides systems, compositions, immunotherapeutic agents and methods for cancer immunotherapy. These compositions provide tunable regulation of gene expression and function in immunotherapy. In one aspect, the systems, compositions, immunotherapeutic agents and other components of the disclosure can be controlled by a separately added stabilizing ligand, which provides a significant flexibility to regulate cancer immunotherapy. Further, the systems, compositions and the methods of the present disclosure may also be combined with therapeutic agents such as chemotherapeutic agents, small molecules, gene therapy, and antibodies.

The tunable nature of the systems and compositions of the disclosure has the potential to improve the potency and duration of the efficacy of immunotherapies. Reversibly silencing the biological activity of adoptively transferred cells using compositions of the present disclosure allows maximizing the potential of cell therapy without irretrievably killing and terminating the therapy.

The present disclosure provides methods for fine tuning of immunotherapy after administration to patients. This in turn improves the safety and efficacy of immunotherapy and increases the subject population that may benefit from immunotherapy.

In some embodiments, immune cells of the disclosure may be T cells, NK cells, antigen presenting cells, for example, a dendritic cell or a tumor infiltrating tumor cell, wherein the immune cell is modified to express CD40L in addition to a second payload, for example, an antigen-specific T cell receptor (TCR), or an antigen specific chimeric antigen receptor (CAR) taught herein (known as CAR T cells). Accordingly, at least one polynucleotide encoding both CD40L and a CAR system (or a TCR), or a first polynucleotide encoding a CD40L payload operably linked to a DRD and a second polynucleotide encoding a different payload, for example, an antigen-specific T cell receptor (TCR), or an antigen specific chimeric antigen receptor (CAR) described herein may be linked to the same or different DRD as the DRD linked to the CD40L. In some embodiments, the tunable protein expression system of the present disclosure may comprise a first polynucleotide encoding a CD40L linked to a first DRD, and a second polynucleotide encoding a second payload, for example, an antigen-specific T cell receptor (TCR), or an antigen specific chimeric antigen receptor (CAR) operably linked to the first DRD or optionally a second, different DRD. The second payload may not be linked to a DRD and may be expressed in the transformed or transfected cell. In various embodiments, the first and second polynucleotide may be present in a single vector, or the two polynucleotides may each be separately present in two different vectors. In some embodiments, when CD40L and the second payload are encoded by the same polynucleotide, the second payload may be operably linked to a DRD or it may be expressed independently of the CD40L and not linked to any DRD, and may be separated from the CD40L and/or DRD by an IRES or some other transcription termination signal, such that the translation and expression of the second payload is independent of the translation and expression of the CD40L payload operably linked to a DRD. The one or more vectors may then be introduced into an immune cell, for example, a T cell, an NK cell, a dendritic cell or a tumor infiltrating tumor cell.

In related embodiments, the T cell expressing the CAR or TCR binds to a specific antigen via the extracellular targeting moiety of the CAR or TCR, thereby a signal via the intracellular signaling domain (s) is transmitted into the T cell, and as a result, the T cell is activated. The activated CAR T cell changes its behavior including release of a cytotoxic cytokine (e.g., a tumor necrosis factor, and lymphotoxin, etc.), improvement of a cell proliferation rate, change in a cell surface molecule, or the like. Such changes cause destruction of a target cell expressing the antigen recognized by the CAR or TCR. In addition, release of a cytokine or change in a cell surface molecule stimulates other immune cells, for example, a B cell, a dendritic cell, a NK cell, and a macrophage.

In related illustrative embodiments, the CAR introduced into a T cell may be a first-generation CAR including only the intracellular signaling domain from TCR CD3zeta, or a second-generation CAR including the intracellular signaling domain from TCR CD3zeta and a costimulatory signaling domain, or a third-generation CAR including the intracellular signaling domain from TCR CD3zeta and two or more costimulatory signaling domains, or a split CAR system, or an on/off switch CAR system. In one example, the expression of the CD40L, CAR or TCR is controlled by the stabilization of an operably linked DRD, which in the absence of a stabilizing ligand will result in the little to no accumulation of payload, i.e. CAR or TCR. In these examples, the two or more payloads may be linked to the same DRD or different DRDs, or one of the two payloads may be unregulated by a DRD.

The payload of interest is operably linked to a DRD, and therefore, without the stabilizing ligand, little to no protein of interest is produced. When stabilizing ligand is administered to the cell transformed with the tunable protein expression system, the DRD-linked payload is stabilized, permitting accumulation of the protein of interest in the cell. In some exemplary embodiments, the presence or absence of the DRD stabilizing ligand is used to tune the CAR or TCR expression in transduced T cells or NK cells. In various embodiments, the payload may be optionally linked to a signal sequence, a leader sequence, a cleavage site or some other peptide or polypeptide sequence or sequences that permits the protein of interest to be separated from the DRD in the cell after its accumulation.

In some embodiments, CAR T cells of the disclosure may be further modified to express another one, two, three or more immunotherapeutic agents. The immunotherapeutic agents may be another CAR or TCR specific to a different target molecule; a cytokine such as IL2, IL12, IL15 and IL18, or a cytokine receptor such as IL15Ra; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed. These molecules may be included in the same effector module or in separate effector modules.

In one embodiment, the CAR T cell (including TCR T cell) of the disclosure may be an “armed” CAR T cell which is transformed with one or more components of the tunable protein expression system comprising a CAR payload and either the same or a different polynucleotide sequence encoding a CD40L operably linked to the same or different DRD. The inducible or constitutively secreted active cytokines further arm CAR T cells to improve efficacy and persistence. In this context, such CAR T cell is also referred to as “armored CAR T cell”. The “armor” molecule may be selected based on the tumor microenvironment and other elements of the innate and adaptive immune systems. In some embodiments, the molecule may be a stimulatory factor such as IL2, IL12, IL15, IL18, type I IFN, CD40L and 4-1BBL which have been shown to further enhance CAR T cell efficacy and persistence in the face of a hostile tumor microenvironment via different mechanisms.

In one embodiment, the tunable protein expression system, and components thereof that tune expression levels and activities of any described payloads or proteins of interest (used interchangeably) may be used for immunotherapy. As non-limiting examples, an immunotherapeutic agent may be an antibody and fragments and variants thereof, a cancer specific T cell receptor (TCR) and variants thereof, an anti-tumor specific chimeric antigen receptor (CAR), a chimeric switch receptor, an inhibitor of a co-inhibitory receptor or ligand, an agonist of a co-stimulatory receptor and ligand, a cytokine, chemokine, a cytokine receptor, a chemokine receptor, a soluble growth factor, a metabolic factor, a suicide gene, a homing receptor, or any agent that induces an immune response in a cell and a subject.

In some embodiments, the composition for inducing or suppressing an immune response may comprise one or more components of a tunable protein expression system, or one or more polypeptides encoded by a tunable protein expression system. In some embodiments, the tunable protein expression system may comprise a first polynucleotide comprising a first nucleic acid sequence that encodes a payload; a second nucleic acid sequence that encodes a drug responsive domain (DRD).

In some embodiments, a tunable protein expression system, and compositions of the present disclosure relate to tunable protein expression (protein of interest or payload) function, including for example, anti-tumor immune responses of immunotherapeutic agents. In some embodiments, the immunotherapeutic agents may include cytokines, chemokines, antibodies, integrins, integral proteins, membrane proteins, extracellular proteins, for example, CD40L, that may be used to upregulate, or improve the function of one or more immune cell types, or down regulate the activity of one or more immune cell types. In various embodiments, the immunotherapeutic agents useful in the treatment of a disease, condition or disorder can include CD40L, alone or in combination with other cytokines, chemokines, antibodies, integrins, integral proteins, membrane proteins, extracellular proteins. In various embodiments, the tunable protein expression system provides a protein of interest or payload that includes CD40L that promotes or upregulates the longevity and activity of one or more immune cell types useful to treat a disease, condition or disorder or a symptom associate with any of these.

2. Adoptive Cell Transfer (Adoptive Immunotherapy)

In some embodiments, cells which are genetically modified to encode and express at least one payload, for example, CD40L operably linked to a DRD, the regulated expression of which may be used for adoptive cell therapy (ACT). As used herein, adoptive cell transfer refers to the administration of immune cells (from autologous, allogenic or genetically modified hosts) with direct anticancer activity. ACT has shown promise in clinical application against malignant and infectious disease.

According to the present disclosure, the one or more components of a tunable protein expression system may be used in the development and implementation of cell therapies such as adoptive cell therapy. In some embodiments, one or more components of a tunable protein expression system, may be used in cell therapies to effect CAR therapies, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

Provided herein are methods for use in adoptive cell therapy. The methods involve preconditioning a subject in need thereof; modulating immune cells with one or more components of a tunable protein expression system, and/or compositions of the present disclosure; administering to a subject engineered immune cells expressing compositions of the disclosure and the successful engraftment of engineered cells within the subject.

In some embodiments, regulatable protein expression systems and compositions of the present disclosure may be used to minimize preconditioning regimens associated with adoptive cell therapy. As used herein “preconditioning” refers to any therapeutic regimen administered to a subject to improve the outcome of adoptive cell therapy. Preconditioning strategies include but are not limited to total body irradiation and/or lymph depleting chemotherapy. Adoptive therapy clinical trials without preconditioning have failed to demonstrate any clinical benefit, indicating its importance in ACT. Yet, preconditioning is associated with significant toxicity and limits the subject cohort that is suitable for ACT. In some instances, immune cells for ACT may be engineered to express CD40 L alone or with a cytokine, such as IL-2, IL-6, IL12 and IL15 as payload using the tunable protein expression constructs described herein to permit selective expression of the protein of interest which may be tuned using a stabilizing ligand of the present disclosure to reduce the need for preconditioning.

In some embodiments, immune cells for ACT may be dendritic cells, T cells such as CD8+ T cells and CD4+ T cells, natural killer (NK) cells, NK T cells, Cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. In other embodiments, immune stimulatory cells for ACT may be generated from embryonic stem cell (ESC) and induced pluripotent stem cell (iPSC). In some embodiments, autologous or allogeneic immune cells are used for ACT.

In some embodiments, cells used for ACT may be antigen presenting cells, for example, dendritic cells and T cells engineered to express CD40L alone or in combination with CARs comprising an antigen-binding domain specific to an antigen on tumor cells of interest. In other embodiments, cells used for ACT may be NK cells engineered to express CD40L alone or in combination with cytokines or CARs which may be used for adoptive immunotherapy. In one example, a mixture of dendritic cells, T cells and/or NK cells may be used for ACT. The expression level of CD40L in antigen presenting cells, T cells and/or NK cells, according to the present disclosure, is tuned and controlled by a small molecule that binds to the DRD(s) operably linked to the payload, for example, CD40L, which enables selective expression of the CD40L in the transformed antigen presenting cells, T cells and NK cells either alone or coupled with other payloads, for example, CARs or cytokines, for example, IL-2, IL-6, IL12 and IL15 as payload.

In some embodiments, NK cells engineered to express one or more components of a tunable protein expression system may be used for ACT. NK cell activation induces perforin/granzyme-dependent apoptosis in target cells. NK cell activation also induces cytokine secretion such as IFN γ, TNF-α and GM-CSF. These cytokines enhance the phagocytic function of macrophages and their antimicrobial activity and augment the adaptive immune response via up-regulation of antigen presentation by antigen presenting cells such as dendritic cells (DCs).

Other examples of genetic modification may include the introduction of chimeric antigen receptors (CARs) and the down-regulation of inhibitory NK cell receptors such as NKG2A.

NK cells may also be genetically reprogrammed to circumvent NK cell inhibitory signals upon interaction with tumor cells. For example, using CRISPR, ZFN, or TALEN to genetically modify NK cells to silence their inhibitory receptors may enhance the anti-tumor capacity of NK cells.

Immune cells can be isolated and expanded ex vivo using a variety of methods known in the art. For example, methods of isolating and expanding cytotoxic T cells are described in U.S. Pat. Nos. 6,805,861 and 6,531,451; US Patent Publication No. US20160348072A1 and International Patent Publication No. WO2016168595A1; the contents of each of which are incorporated herein by reference in their entirety. Isolation and expansion of NK cells is described in US Patent Publication No. US20150152387A1, U.S. Pat. No. 7,435,596; and Oyer, J. L. (2016). Cytotherapy 18(5):653-63; the contents of each of which are incorporated by reference herein in its entirety. Specifically, human primary NK cells may be expanded in the presence of feeder cells e.g. a myeloid cell line that has been genetically modified to express membrane bound IL15, IL21, IL12 and 4-1BBL.

In some instances, sub populations of immune cells may be enriched for ACT. Methods for immune cell enrichment are taught in International Patent Publication No. WO2015039100A1. In another example, T cells positive for B and T lymphocyte attenuator marker BTLA) may be used to enrich for T cells that are anti-cancer reactive as described in U.S. Pat. No. 9,512,401 (the content of each of which are incorporated herein by reference in their entirety).

In some embodiments, immune cells for ACT may be depleted of select sub populations to enhance T cell expansion. For example, immune cells may be depleted of Foxp3+T lymphocytes to minimize the anti-tumor immune response using methods taught in US Patent Publication No. US 20160298081A1; the contents of which are incorporated by reference herein in their entirety.

In some embodiments, activation and expansion of T cells for ACT is achieved antigenic stimulation of a transiently expressed Chimeric Antigen Receptor (CAR) on the cell surface. Such activation methods are taught in International Patent NO. WO2017015427, the content of which are incorporated herein by reference in their entirety.

In some embodiments, immune cells may be activated by antigens associated with antigen presenting cells (APCs). In some embodiments, the APCs may be dendritic cells, macrophages or B cells that are antigen specific or nonspecific. The APCs may autologous or homologous in their organ. In some embodiments, the APCs may be artificial antigen presenting cells (aAPCs) such as cell based aAPCs or acellular aAPCs. Cell based aAPCs may be selected from either genetically modified allogeneic cells such as human erythroleukemia cells or xenogeneic cells such as murine fibroblasts and Drosophila cells. Alternatively, the APCs maybe be acellular wherein the antigens or costimulatory domains are presented on synthetic surfaces such as latex beads, polystyrene beads, lipid vesicles or exosomes.

In some embodiments, cells of the disclosure, specifically T cells may be expanded using artificial cell platforms. In one embodiment, the mature T cells may be generated using artificial thymic organoids (ATOS) described by Seet C S et al. 2017. Nat Methods 14, 521-530 (the contents of which are incorporated herein by reference in their entirety). ATOs are based on a stromal cell line expressing delta like canonical notch ligand (DLL1). In this method, stromal cells are aggregated with hematopoietic stem and progenitor cells by centrifugation and deployed on a cell culture insert at the air-fluid interface to generate organoid cultures. ATO-derived T cells exhibit naive phenotypes, a diverse T cell receptor (TCR) repertoire and TCR-dependent function.

In some embodiments, adoptive cell therapy is carried out by autologous transfer, wherein the cells are derived from a subject in need of a treatment and the cells, following isolation and processing are administered to the same subject. In other instances, ACT may involve allogenic transfer wherein the cells are isolated and/or prepared from a donor subject other than the recipient subject who ultimately receives cell therapy. The donor and recipient subject may be genetically identical, or similar or may express the same HLA class or subtype.

In some embodiments, the multiple immunotherapeutic agents introduced into the immune cells for ACT (e.g., dendritic cells, T cells and NK cells) may be controlled by the same or different tunable protein expression systems. In one example, each of the two payloads, for example, a CD40L payload and a CAR construct such as CD19 CAR payload are regulated by one or more DRDs on the same or different tunable protein expression systems. In some related embodiments, the payloads are linked to the same or different DRDs. In some embodiments, the CD40L is operably linked to a DRD, and the CAR construct such as CD19 CAR is positioned upstream or down stream from the CD40L and not linked to any DRD, or the CAR construct such as CD19 CAR is introduced into the cell encoded by a separate nucleotide sequence as the nucleotide sequence encoding the first payload, and the second payload may be linked to a DRD which is the same or different from the CD40L linked DRD or may be free of any linkage to any DRD. The payloads are transcribed, translated and expressed when the DRD(s) is/are stabilized with a stabilizing ligand specific for the DRD(s). The expression of CD40L and optionally a second payload, for example, IL12 and/or CD19 CAR may be tuned using one or more stabilizing ligands. In other embodiments, the multiple immunotherapeutic agents introduced into the immune cells for ACT (e.g., T cells and NK cells) may be controlled by different tunable protein expression systems. In one example, CD40L and a CAR construct such as CD19 CAR, each may be operably linked to different DRDs, and thereby can be tuned separately using different stimuli.

Following genetic modulation using one or more components of a tunable protein expression system and compositions of the disclosure, cells are administered to the subject in need thereof. Methods for administration of cells for adoptive cell therapy are known and may be used in connection with the provided methods and compositions.

In some embodiments, immune cells for ACT may be modified to express one or more immunotherapeutic agents (proteins of interest) which facilitate immune cells activation, infiltration, expansion, survival and anti-tumor functions. The immunotherapeutic agents may be a second CAR or TCR specific to a different target molecule; a cytokine or a cytokine receptor; a chimeric switch receptor that converts an inhibitory signal to a stimulatory signal; a homing receptor that guides adoptively transferred cells to a target site such as the tumor tissue; an agent that optimizes the metabolism of the immune cell; or a safety switch gene (e.g., a suicide gene) that kills activated T cells when a severe event is observed after adoptive cell transfer or when the transferred immune cells are no-longer needed.

In some embodiments, immune cells used for adoptive cell transfer can be genetically manipulated to improve their persistence, cytotoxicity, tumor targeting capacity, and ability to home to disease sites in vivo, with the overall aim of further improving upon their capacity to kill tumors in cancer patients. One example is to introduce one or more components of a tunable protein expression system of the disclosure encoding a cytokine, such as a gamma-cytokine (e.g. IL2 and IL15) into immune cells to promote immune cell proliferation and survival. Transduction of cytokine genes (e.g., gamma-cytokines IL2 and IL15) encoded by a tunable protein expression system into immune cells will enable the immune cells, e.g. NK cells to propagate without addition of exogenous cytokines such that the cytokine expressing NK cells have enhanced tumor cytotoxicity.

In some embodiments, one or more components of a tunable protein expression system may be utilized to prevent T cell exhaustion. As used herein, “T cell exhaustion” refers to the stepwise and progressive loss of T cell function caused by chronic T cell activation. T cell exhaustion is a major factor limiting the efficacy of antiviral and antitumor immunotherapies. Exhausted T cells have low proliferative and cytokine producing capabilities concurrent with high rates of apoptosis and high surface expression of multiple inhibitory receptors. T cell activation leading to exhaustion may occur either in the presence or absence of the antigen.

In some embodiments, the tunable protein expression system and their components may be utilized to prevent T cell exhaustion in the context of Chimeric Antigen Receptor-T cell therapy (CAR-T). In this context, exhaustion in some instances, may be caused by the oligomerization of the scFvs of the CAR on the cell surface which leads to continuous activation of the intracellular domains of the CAR. As a non-limiting example, CARs of the present disclosure may include scFvs that are unable to oligomerize. As another non-limiting example, CARs that are rapidly internalized and re-expressed following antigen exposure may also be selected to prevent chronic scFv oligomerization on cell surface. In one embodiment, the framework region of the scFvs may be modified to prevent constitutive CAR signaling (Long et al. 2014. Cancer Research. 74(19) S1; the contents of which are incorporated by reference in their entirety). One or more components of a tunable protein expression system of the present disclosure may also be used to regulate the surface expression of the CAR on the T cell surface to prevent chronic T cell activation. The CARs of the disclosure may also be engineered to minimize exhaustion. As a non-limiting example, the 41-BB signaling domain may be incorporated into CAR design to ameliorate T cell exhaustion. In some embodiments, any of the strategies disclosed by Long H A et al. may be utilized to prevent exhaustion (Long A H et al. (2015) Nature Medicine 21, 581-590; the contents of which are incorporated herein by reference in their entirety).

In some embodiments, the tunable nature of the tunable protein expression system of the present disclosure may be utilized to reverse human T cell exhaustion observed with tonic CAR signaling. Reversibly silencing the biological activity of adoptively transferred cells using compositions of the present disclosure may be used to reverse tonic signaling which, in turn, may reinvigorate the T cells. Reversal of exhaustion may be measured by the downregulation of multiple inhibitory receptors associated with exhaustion.

In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion (Long et al. Journal for Immunotherapy of Cancer 2013, 1 (Suppl 1): P21; the contents of which are incorporated by reference in their entirety). In one embodiment, T cells of the present disclosure may be used in combination with inhibitors of glycolysis such as 2-deoxyglucose, and rapamycin.

In some embodiments, payloads or proteins of interest of the disclosure may be used in conjunction with antibodies or fragments that target T cell surface markers associated with T cell exhaustion. T-cell surface markers associated with T cell exhaustion that may be used include, but are not limited to, CTLA-1, PD-1, TGIT, LAG-3, 2B4, BTLA, TIM3, VISTA, and CD96. In some embodiments, one or more components of a tunable protein expression system may be utilized to prevent T cell exhaustion. As used herein, “T cell exhaustion” refers to the stepwise and progressive loss of T cell function caused by chronic T cell activation. T cell exhaustion is a major factor limiting the efficacy of antiviral and antitumor immunotherapies. Exhausted T cells have low proliferative and cytokine producing capabilities concurrent with high rates of apoptosis and high surface expression of multiple inhibitory receptors. T cell activation leading to exhaustion may occur either in the presence or absence of the antigen.

In some embodiments, one or more components of a tunable protein expression system, and their components may be utilized to prevent T cell exhaustion in the context of Chimeric Antigen Receptor-T cell therapy (CAR-T). In this context, exhaustion in some instances, may be caused by the oligomerization of the scFvs of the CAR on the cell surface which leads to continuous activation of the intracellular domains of the CAR. As a non-limiting example, CARs of the present disclosure may include scFvs that are unable to oligomerize. As another non-limiting example, CARs that are rapidly internalized and re-expressed following antigen exposure may also be selected to prevent chronic scFv oligomerization on cell surface. In one embodiment, the framework region of the scFvs may be modified to prevent constitutive CAR signaling (Long et al. 2014. Cancer Research. 74(19) S1; the contents of which are incorporated by reference in their entirety). One or more components of a tunable protein expression system of the present disclosure may be also used to regulate the surface expression of the CAR on the T cell surface to prevent chronic T cell activation. The CARs of the disclosure may also be engineered to minimize exhaustion. As a non-limiting example, the 41-BB signaling domain may be incorporated into CAR design to ameliorate T cell exhaustion.

In some embodiments, the compositions of the present disclosure may be utilized to alter TIL (tumor infiltrating lymphocyte) populations in a subject. In one embodiment, any of the payloads described herein may be utilized to change the ratio of CD4 positive cells to CD8 positive populations. In some embodiments, TILs may be sorted ex vivo and engineered to express any of the cytokines described herein. Payloads of the disclosure may be used to expand CD4 and/or CD8 populations of TILs to enhance TIL mediated immune response.

Parameters for improving CAR-T therapy outcome are described in Finney et al. JCI. 2019; 129(5):2123-2132 (the contents of which are herein incorporated by reference in their entirety). The levels of biomarker LAG3 (high)/TNF-α (low) in peripheral blood CD8+ T cells at the time of apheresis may also predict a subsequent dysfunctional response in subjects with high antigen load who do not achieve complete response that is durable for more than a few weeks. T cell-intrinsic features that are a consequence of the starting T cell repertoire and the effects of the manufacturing process converge with CD19 antigen-induced activation following adoptive transfer may also play a role in the outcome of CAR-T therapy. The starting T cell repertoire may in part be affected by the timing of the apheresis. In one embodiment, the apheresis may be performed prior to chemotherapy. Cumulative burden of CD19 expressing leukemic and normal B cells, as evaluated in the bone marrow prior to lymph depleting chemotherapy may be important for determining CAR-T therapy outcome. According to Finney et al., increase antigen burden improves CAR-T therapy outcome. To increase CD19 antigen burden in vivo, subjects may also be infused with expanded subject derived T cells genetically modified to express CD19 (also referred to as T-APCs).

3. Cancer Vaccines

In some embodiments, tunable protein expression system constructs, payloads of interest (e.g., immunotherapeutic agents), vectors, cells and compositions of the present disclosure may be used in conjunction with cancer vaccines.

In some embodiments, cancer vaccine may comprise peptides and/or proteins derived from tumor associated antigen (TAA). Such strategies may be utilized to evoke an immune response in a subject, which in some instances may be a cytotoxic T lymphocyte (CTL) response. Peptides used for cancer vaccines may also modified to match the mutation profile of a subject. For example, EGFR derived peptides with mutations matched to the mutations found in the subject in need of therapy have been successfully used in patients with lung cancer.

In one embodiment, cancer vaccines of the present disclosure may include superagonist altered peptide ligands (APL) derived from TAAs. These are mutant peptide ligands deviate from the native peptide sequence by one or more amino acids, which activate specific CTL clones more effectively than native epitopes. These alterations may allow the peptide to bind better to the restricting Class I MHC molecule or interact more favorably with the TCR of a given tumor-specific CTL subset. APLs may be selected using methods known in the art.

In some embodiments, effector immune cells genetically modified to encode the components of the tunable protein expression system, and payloads of the disclosure may be combined with the biological adjuvants described herein. Dual regulation of CAR and cytokines and ligands to segregate the kinetic control of target-mediated activation from intrinsic cell T cell expansion. Such dual regulation also minimizes the need for pre-conditioning regimens in patients. As a non-limiting example, a DRD regulated payload, for example, a CD40L, in combination with a CAR e.g. CD19 CAR may be combined with cytokines e.g. IL12 to enhance the anti-tumor efficacy of the CAR. As another non-limiting example, dendritic cell-based vaccinations combined with recombinant human IL7 to improve outcome in high-risk pediatric sarcomas patients may be employed in the methods described herein.

In some embodiments, effector immune cells modified to express one or more antigen-specific TCRs or CARs may be combined with compositions of the disclosure comprising immunotherapeutic agents, for example, CD40L that convert the immunosuppressive tumor microenvironment.

In one aspect, effector immune cells modified to express CARs specific to different target molecules on the same cell may be combined. In another aspect, different immune cells modified to express the same CAR construct such as NK cells and T cells may be used in combination for a tumor treatment, for instance, a T cell modified to express a CD40L in combination with a CD19 CAR may be combined with a NK cell modified to express the same CD19 CAR to treat B cell malignancy.

In other embodiments, immune cells modified to express CARs may be combined with checkpoint blockade agents.

In some embodiments, effector immune cells genetically modified to express one or more components of the tunable protein expression system, for example a payload of the disclosure, may be combined with cancer vaccines and other immunotherapeutics and adjuvant treatments of the disclosure.

In some embodiments, methods of the disclosure may include combination of the compositions of the disclosure with other agents effective in the treatment of cancers, infection diseases and other immunodeficient disorders, such as anti-cancer agents. As used herein, the term “anti-cancer agent” refers to any agent which is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In some embodiments, anti-cancer agent or therapy may be a chemotherapeutic agent, or radiotherapy, immunotherapeutic agent, surgery, or any other therapeutic agent which, in combination with the present disclosure, improves the therapeutic efficacy of treatment.

In one embodiment, one or more components of a tunable protein expression system comprising a CD19 CAR may be used in combination with amino pyrimidine derivatives such as the Burkit's tyrosine receptor kinase (BTK) inhibitor.

In some embodiments, compositions of the present disclosure may be used in combination with immunotherapeutics other than the inventive therapy described herein, such as antibodies specific to some target molecules on the surface of a tumor cell.

Exemplary chemotherapies include, without limitation, Acivicin; Aclarubicin; Acodazole hydrochloride; Acronine; Adozelesin; Aldesleukin; Altretamine; Ambomycin; Ametantrone acetate; Amsacrine; Anastrozole; Anthramycin; Asparaginase; Asperrin, Sulindac, Curcumin, alkylating agents including: Nitrogen mustards such as mechlor-ethamine, cyclophosphamide, ifosfamide, melphalan and chlorambucil; nitrosoureas such as carmustine (BC U), lomustine (CCNU), and semustine (methyl-CC U); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrrolidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIFf) and procarbazine, adrenocortical suppressants such as mitotane (o,ρ′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; anti-virals such as oseltamivir phosphate, Amphotericin B, and palivizumab; Sdi 1 mimetics; Semustine; Senescence derived inhibitor 1; Sparfosic acid; Spicamycin D; Spiromustine; Splenopentin; Spongistatin 1; Squalamine; Stipiamide; Stromelysin inhibitors; Sulfinosine; Superactive vasoactive intestinal peptide antagonist; Velaresol; Veramine; Verdins; Verteporfin; Vinorelbine; Vinxaltine; Vitaxin; Vorozole; Zanoterone; Zeniplatin; Zilascorb; and Zinostatin stimalamer; PI3Kβ small-molecule inhibitor, GSK2636771; pan-PI3K inhibitor (BKM120); BRAF inhibitors. Vemurafenib (Zelboraf) and dabrafenib (Tafinlar); or any analog or derivative and variant of the foregoing.

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the chemotherapeutic agent may be an immunomodulatory agent such as lenalidomide (LEN). Recent studies have demonstrated that lenalidomide can enhance antitumor functions of CAR modified T cells. Some examples of anti-tumor antibodies include tocilizumab, siltuximab.

Other agents may be used in combination with compositions of the disclosure may also include, but not limited to, agents that affect the upregulation of cell surface receptors and their ligands such as Fas/Fas ligand, DR4 or DR5/TRAIL and GAP junctions, cytostatic and differentiation agents, inhibitors of cell adhesion such as focal adhesion kinase (FAKs) inhibitors and Lovastatin, or agents that increase the sensitivity of the hyper proliferative cells to apoptotic inducers such as the antibody C225.

The combinations may include administering the compositions of the disclosure and other agents at the same time or separately. Alternatively, the present immunotherapy may precede or follow the other agent/therapy by intervals ranging from minutes, days, weeks to months.

4. Diseases

Provided in the present disclosure is a method of reducing a tumor volume or burden in a subject in need, the method comprising introducing into the subject a composition of the disclosure.

The present disclosure also provides methods for treating a cancer in a subject, comprising administering to the subject an effective amount of effector immune cells genetically modified to comprise a tunable protein expression system of the present disclosure.

5. Cancer

Various cancers may be treated with pharmaceutical compositions, tunable protein expression system components, and constructs including their DRDs and payloads of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of sarcomas which may be treated with the compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

6. Infectious Diseases

In some embodiment, tunable protein expression system of the disclosure may be used for the treatment of infectious diseases. Tunable protein expression systems of the disclosure may be introduced in cells suitable for adoptive cell transfer such as macrophages, dendritic cells, natural killer cells, and or T cells. Infectious diseases treated by the tunable protein expression system of the disclosure may include diseases caused by viruses, bacteria, fungi, and/or parasites. IL15-IL15Ra payloads of the disclosure may be used to increase immune cell proliferation and/or persistence of the immune cells useful in treating infectious diseases.

“Infectious diseases” herein refer to diseases caused by any pathogen or agent that infects mammalian cells, preferably human cells and causes a disease condition. Examples thereof include bacteria, yeast, fungi, protozoans, mycoplasma, viruses, prions, and parasites. Examples include those involved in (a) viral diseases such as, for example, diseases resulting from infection by an adenovirus, a herpesvirus (e.g., HSV-I, HSV-II, CMV, or VZV), a poxvirus (e-g-, an orthopoxvirus such as variola or vaccinia, or molluscum contagiosum), a picornavirus (e.g., rhinovirus or enterovirus), an orthomyxovirus (e.g., influenza virus), a paramyxovirus (e.g., parainfluenza virus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such chlamydia, fungal diseases including but not limited to candidiasis, aspergillosis, histoplasmosis, cryptococcal meningitis, parasitic diseases including but not limited to malaria, Pneumocystis carnii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Straüssler-Scheinker syndrome, Fatal Familial Insomnia and kuru.

7. Immuno-Oncology and Cell Therapies

Recent progress in the field of cancer immunology has allowed the development of several approaches to help the immune system keep the cancer at bay. Such immunotherapy approaches include the targeting of cancer antigens through monoclonal antibodies or through adoptive transfer of ex vivo engineered T cells (e.g., which contain chimeric antigen receptors or engineered T cell receptors).

In some embodiments, pharmaceutical compositions, tunable protein expression systems of the present disclosure may be used in the modulation or alteration or exploitation of the immune system to target one or more cancers. This approach may also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the pharmaceutical compositions, tunable protein expression systems, including their payloads of the present disclosure.

Cancer immunotherapy refers to a diverse set of therapeutic strategies designed to induce the patient's own immune system to fight the cancer. In some embodiments, pharmaceutical compositions, pharmaceutical compositions, tunable protein expression systems, including their payloads of the present disclosure are designed as immune-oncology therapeutics.

8. Cell Therapies

There are several types of cellular immunotherapies, including tumor infiltrating lymphocyte (TIL) therapy, genetically engineered T cells bearing chimeric antigen receptors (CARs), and recombinant TCR technology.

According to the present disclosure, the tunable protein expression system may be used in the development and implementation of cell therapies such as adoptive cell therapy. The tunable protein expression systems, and their payloads may be used in cell therapies to effect TCR removal-TCR gene disruption, TCR engineering, to regulate epitope tagged receptors, in APC platforms for stimulating T cells, as a tool to enhance ex vivo APC stimulation, to improve methods of T cell expansion, in ex vivo stimulation with antigen, in TCR/CAR combinations, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

In some embodiments, improved response rates are obtained in support of cell therapies.

Expansion and persistence of cell populations may be achieved through regulation or fine tuning of the payloads, e.g., the receptors or pathway components in T cells, NK cells or other immune-related cells. In some embodiments, tunable protein expression systems of the present disclosure are designed to spatially and/or temporally control the expression of proteins which enhance T-cell or NK cell responses. In some embodiments, tunable protein expression systems are designed to spatially and/or temporally control the expression of proteins which inhibit T-cell or NK cell response.

The immune system can be harnessed for the treatment of diseases beyond cancer. Tunable protein expression systems, their components may be utilized in immunotherapy for the treatment of diseases including, but not limited to, autoimmune diseases, allergies, graft versus host disease, and diseases and disorders that may result in immunodeficiency such as acquired immune deficiency syndrome (AIDS).

The present disclosure provides compositions for immunotherapy. Such compositions may include effector modules with CD40L as the payload. The compositions may further include a chimeric antigen receptor as an additional payload. The compositions may be expressed in immune cells suitable for adoptive cell therapy. Cells expressing the effector modules may be delivered to a subject directly. In some aspects, the cells expressing the effector modules may be co-administered with immune cells expressing CD40. CD40 expression in these cells may be ectopic or endogenous. CD40 expression may also be induced by co-culture with other immune cells expressing the effector modules of the invention disclosure. Some non-limiting examples of CD40 positive cell include cell such as dendritic cells, macrophages, myeloid cell, B cells, platelets, endothelial cells, epithelial cells, and fibroblasts.

In some embodiments, the payloads described herein may be used for dendritic cell activation. In some embodiments, the dendritic cell may be a myeloid dendritic cell, a plasmacytoid dendritic cell, a CD14+ dendritic cell, a Langerhans cell, or a microglia. In some embodiments, myeloid DCs (mDCs) express typical myeloid antigens CD11c, CD13, CD33 and CD11b, corresponding to mouse CD11c+“classical” or “conventional” DCs. In humans both monocytes and mDCs express CD11c, but DCs lack CD14 or CD16 and may be split into CD1c+ and CD141+ fractions. These two fractions share homology with mouse classical DCs expressing either CD11b (CD1c+ DCs) or CD8/CD103 (CD141+ DCs). In some embodiments, dendritic cells may be plasmacytoid dendritic cells (pDCs) plasmacytoid DCs (pDCs) typically lack myeloid antigens and may be distinguished by expression of CD123, CD303 and CD304. In one embodiment, the dendritic cells may be CD14+. Such cells are found in tissues and lymph nodes are a third subset of CD11c+ myeloid cells originally described as ‘interstitial DCs’. They are more monocyte-like or macrophage-like than CD1c+ and CD141+ mDCs and may arise from classical monocytes. Equivalent cells have recently been found in mice as a new monocyte-derived subset of CD11b classical DCs that expresses or ESAM. In one embodiment, the dendritic cells may be Langerhans cells or microglia. Langerhans cells (LCs) and microglia are two specialized self-renewing DC populations found in stratified squamous epithelium and parenchyma of the brain, respectively. The LCs may be capable of differentiating into migratory DCs whereas microglia are considered as a type of macrophage.

In some embodiments, payloads of the present disclosure may be a chimeric antigen receptor (CAR), which when transduced into immune cells (e.g., T cells and NK cells), can re-direct the immune cells against the target (e.g., a tumor cell) which expresses a molecule recognized by the extracellular target moiety of the CAR.

In some embodiments, pharmaceutical compositions comprising a tunable protein expression system, including their payloads or protein of interest may be used in the modulation or alteration or exploitation of the immune system to target one or more self-reactive immune components such as auto antibodies and self-reactive immune cells to attenuate autoimmune diseases.

In some embodiments, tunable protein expression systems may be utilized in immunotherapy-based treatments to attenuate or mitigate Graft vs. Host disease (GVHD). GVHD refers to a condition following stem cell or bone marrow transplant where in the allogeneic donor immune cells react against host tissue. In some embodiments, a tunable protein expression system may be designed to encode a cytokine or immunological agent designed to modulate Tregs for the treatment of GVHD.

In some embodiments, tunable protein expression systems may be significantly less immunogenic than other biocircuits or switches in the art due to the expression of human native proteins of interest.

Various autoimmune diseases and autoimmune-related diseases may be treated with pharmaceutical compositions comprising a tunable protein expression systems of the present disclosure. As used herein, the term “autoimmune disease” refers to a disease in which the body produces antibodies that attack its own tissues.

Various blood diseases may be treated with pharmaceutical compositions comprising one or more components of a tunable protein expression system of the present disclosure.

9. Central Nervous System (CNS)

In some embodiments, pharmaceutical compositions comprising one or more components of a tunable protein expression system of the present disclosure may be used in the modulation or alteration or exploitation of proteins in the central nervous system including cerebrospinal (CSF) proteins.

10. Stem Cell Applications

The tunable protein expression system of the present disclosure and/or their components may be utilized in the regulated reprogramming of cells, stem cell engraftment or other application where controlled or tunable expression of such reprogramming factors are useful.

The tunable protein expression system constructs of the present disclosure may be used in reprogramming cells including stem cells or induced stem cells. Induction of induced pluripotent stem cells (iPSC) was first achieved by Takahashi and Yamanaka (Cell, 2006. 126(4):663-76; herein incorporated by reference in its entirety) using viral vectors to express KLF4, c-MYC, OCT4 and SOX2 otherwise collectively known as KMOS.

Excisable lentiviral and transposon vectors, repeated application of transient plasmid, episomal and adenovirus vectors have also been used to try to derive iPSC.

DNA-free methods to generate human iPSC has also been derived using serial protein transduction with recombinant proteins incorporating cell-penetrating peptide moieties, and infectious transgene delivery using the Sendai virus.

The tunable protein expression system of the present disclosure may include a payload comprising any of the genes including, but not limited to, OCT such as OCT4, SOX such as SOX1, SOX2, SOX3, SOX15 and SOX18, NANOG, KLF such as KLF1, KLF2, KLF4 and KLF5, MYC such as c-MYC and n-MYC, REM2, TERT and LIN28 and variants thereof in support of reprogramming cells. Sequences of such reprogramming factors are taught in for example International Application PCT/US2013/074560, the contents of which are incorporated herein by reference in their entirety.

The tunable protein expression system of the present disclosure may include a payload comprising any of factors that contribute stem cell mobilization. In autologous stem cell therapy, sources of stem cells for transplantation may include the bone marrow, peripheral blood mononuclear cells and cord blood. Stem cells are stimulated out of these sources (e.g., the bone marrow) into the blood stream. So sufficient stem cells are available for collection for future reinfusion. One or a combination of cytokines strategies may be used to mobilize the stem cells including but not limited to G-CSF (filgrastim), GM-CSF, and chemotherapy preceding with cytokines (chemomobilization).

11. Tools and Agents for Making Therapeutics

Provided in the present disclosure are tools and agents that may be used in generating therapeutics such as, but not limited to, immunotherapeutics for reducing a tumor volume or burden in a subject in need. A considerable number of variables are involved in producing a therapeutic agent, such as structure of the payload, type of cells, method of gene transfers, method and time of ex vivo expansion, pre-conditioning and the amount and type of tumor burden in the subject. Such parameters may be optimized using tools and agents described herein.

12. Cell Lines

The present disclosure provides a mammalian cell that has been genetically modified with the compositions of the disclosure. Suitable mammalian cells include primary cells and immortalized cell lines. Suitable mammalian cell lines include but are not limited to Human embryonic kidney cell line 293, fibroblast cell line NIH 3T3, human colorectal carcinoma cell line HCT116, ovarian carcinoma cell line SKOV-3, immortalized T cell lines (e.g. Jurkat cells and SupT1 cells), lymphoma cell line Raji cells, NALM-6 cells, K562 cells, HeLa cells, PC12 cells, HL-60 cells, NK cell lines (e.g. NKL, NK92, NK962, and YTS), and the like. In some instances, the cell is not an immortalized cell line, but instead a cell obtained from an individual and is herein referred to as a primary cell. For example, the cell is a T lymphocyte obtained from an individual. Other examples include, but are not limited to cytotoxic cells, stem cells, peripheral blood mononuclear cells or progenitor cells obtained from an individual.

13. Cellular Assays

In some embodiments, the effectiveness of the compositions of the disclosures as immunotherapeutic agents may be evaluated using cellular assays. Levels of expression and/or identity of the compositions of the disclosure may be determined according to any methods known in the art for identifying proteins and/or quantitating proteins levels. In some embodiments, such methods may include Western Blotting, flow cytometry, and immunoassays.

Provided herein are methods for functionally characterizing cells transformed or transduced with a tunable protein expression system construct of the present disclosure and compositions of the disclosure. In some embodiments, functional characterization is carried out in primary immune cells or immortalized immune cell lines and may be determined by expression of cell surface markers. Examples of cell surface markers for T cells include, but are not limited to, CD3, CD4, CD8, CD 14, CD20, CD11b, CD16, CD45 and HLA-DR, CD 69, CD28, CD44, IFNgamma. Markers for T cell exhaustion include PD1, TIM3, BTLA, CD160, 2B4, CD39, and LAG3. Examples of cell surface markers for antigen presenting cells include, but are not limited to, MHC class I, MHC Class II, CD40, CD45, B7-1, B7-2, IFN γ receptor and IL2 receptor, ICAM-1 and/or Fcγ receptor. Examples of cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or Dectin-1 and the like; while in some cases also having the absence of CD2, CD3, CD4, CD8, CD14, CD15, CD16, CD 19, CD20, CD56, and/or CD57. Examples of cell surface markers for NK cells include, but are not limited to, CCL3, CCL4, CCL5, CCR4, CXCR4, CXCR3, NKG2D, CD71, CD69, CCR5, Phospho JAK/STAT, phospho ERK, phospho p38/MAPK, phospho AKT, phospho STAT3, Granulysin, Granzyme B, Granzyme K, IL10, IL22, IFNg, LAP, Perforin, and TNFa.

In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion. In one embodiment, T cells of the present disclosure may be used in combination with inhibitors of glycolysis such as 2-deoxyglucose, and rapamycin.

In some embodiments, tunable protein expression system constructs of the present disclosure, useful for immunotherapy may be placed under the transcriptional control of the T cell receptor alpha locus constant (TRAC) locus in the T cells. Eyquem et al. have shown that expression of the CAR from the TRAC locus prevents T cell exhaustion and the accelerated differentiation of T cells caused by excessive T cell activation.

In some embodiments, payloads of the disclosure may include, antibodies or fragments that target T cell surface markers associated with T cell exhaustion. T-cell surface markers associated with T cell exhaustion that may be used as payloads include, but are not limited to, CTLA-1, PD-1, TGIT, LAG-3, 2B4, BTLA, TIM3, VISTA, and CD96.

In one embodiment, the payload of the disclosure may be a CD276 CAR (with CD28, 4-IBB, and CD3 zeta intracellular domains), that does not show an upregulation of the markers associated with early T cell exhaustion.

14. Cells

In accordance with the present disclosure, cells genetically modified to express at least one protein of interest or payload under the regulation of the encoded DRD ligand of the disclosure are provided. Cells of the disclosure may include, without limitation, immune cells, stem cells and tumor cells. In some embodiments, immune cells are effector immune cells, including, but not limiting to, T cells such as CD8+ T cells and CD4+ T cells (e.g., Th1, Th2, Th17, Foxp3+ cells), memory T cells such as T memory stem cells, central T memory cells, and effector memory T cells, terminally differentiated effector T cells, natural killer (NK) cells, NK T cells, tumor infiltrating lymphocytes (TILs), cytotoxic T lymphocytes (CTLs), regulatory T cells (Tregs), and dendritic cells (DCs, for example, a myeloid dendritic cell, a plasmacytoid dendritic cell, a CD14+ dendritic cell, a Langerhans cell, or a microglia), other immune cells that can elicit an effector function, or the mixture thereof. T cells may be Tαβ cells and Tγδ cells. In some embodiments, stem cells may be from human embryonic stem cells, mesenchymal stem cells, and neural stem cells. In some embodiments, T cells may be depleted endogenous T cell receptors.

In some embodiments, cells of the disclosure may be autologous, allogeneic, syngeneic, or xenogeneic in relation to a particular individual subject.

In some embodiments, cells of the disclosure may be mammalian cells, particularly human cells. Cells of the disclosure may be primary cells or immortalized cell lines.

Engineered immune cells can be accomplished by method comprising introducing into a cell a nucleic acid molecule comprising: a first nucleic acid sequence that encodes at least one payload operably linked to second nucleic acid sequence that encodes a drug responsive domain (DRD).

The vector may be a viral vector such as a lentiviral vector, a gamma-retroviral vector, a recombinant AAV, an adenoviral vector and an oncolytic viral vector. In other aspects, non-viral vectors for example, nanoparticles and liposomes may also be used. In some embodiments, immune cells of the disclosure are genetically modified to express at least one immunotherapeutic agent of the disclosure which is tunable using a stabilizing ligand. In some examples, two, three or more immunotherapeutic agents constructed in the same tunable protein expression system constructs are introduced into a cell. In other examples, two, three, or more tunable protein expression system constructs may be introduced into a cell.

In some embodiments, immune cells of the disclosure may be T cells and/or NK cells modified to express a CD40L and optionally, in combination with an antigen-specific T cell receptor (TCR), or an antigen specific chimeric antigen receptor (CAR) taught herein.

15. Polynucleotides

Tunable protein expression system components including effector modules, their SREs and payloads, may be nucleic acid-based. The term “nucleic acid,” in its broadest sense, includes any compound and/or substance that comprise a polymer of nucleotides, e.g., linked nucleosides. These polymers are often referred to as polynucleotides. Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In some embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Polynucleotides of the disclosure may be mRNA or any nucleic acid molecule and may or may not be chemically modified.

Traditionally, the basic components of an mRNA molecule include at least a coding region, a 5′UTR, a 3′UTR, a 5′ cap and a poly-A tail. Building on this wild type modular structure, the present disclosure expands the scope of functionality of traditional mRNA molecules by providing payload constructs which maintain a modular organization, but which comprise one or more structural and/or chemical modifications or alterations which impart useful properties to the polynucleotide, for example tunability of function. As used herein, a “structural” feature or modification is one in which two or more linked nucleosides are inserted, deleted, duplicated, inverted or randomized in a polynucleotide without significant chemical modification to the nucleosides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, polynucleotides of the present disclosure may harbor 5′UTR sequences which play a role in translation initiation. 5′UTR sequences may include features such as Kozak sequences which are commonly known to be involved in the process by which the ribosome initiates translation of genes, Kozak sequences have the consensus XCCR(A/G) CCAUG, where R is a purine (adenine or guanine) three bases upstream of the start codon (AUG) and X is any nucleotide. In one embodiment, the Kozak sequence is ACCGCC. By engineering the features that are typically found in abundantly expressed genes of target cells or tissues, the stability and protein production of the polynucleotides of the disclosure can be enhanced.

Further provided are polynucleotides, which may contain an internal ribosome entry site (IRES) which play an important role in initiating protein synthesis in the absence of 5′ cap structure in the polynucleotide. An IRES may act as the sole ribosome binding site, or may serve as one of the multiple binding sites. Polynucleotides of the disclosure containing more than one functional ribosome binding site may encode several peptides or polypeptides that are translated independently by the ribosomes giving rise to bicistronic and/or multicistronic nucleic acid molecules.

In one embodiment, polynucleotides of the present disclosure may encode variant polypeptides which have a certain identity with a reference polypeptide sequence. As used herein, a “reference polypeptide sequence” refers to a starting polypeptide sequence. Reference sequences may be wild type sequences or any sequence to which reference is made in the design of another sequence.

The term “identity” as known in the art, refers to a relationship between two or more sequences, as determined by comparing the sequences. In the art, identity also means the degree of sequence relatedness between sequences, as determined by the number of matches between strings of two or more residues (amino acid or nucleic acid). Identity measures the percent of identical matches between two or more sequences with gap alignments (if any) addressed by a particular mathematical model or computer program (i.e., “algorithms”). Identity of related sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the variant sequence may have the same or a similar activity as the reference sequence. Alternatively, the variant may have an altered activity (e.g., increased or decreased) relative to a reference sequence. Generally, variants of a particular polynucleotide or polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schïffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.)

16. Codon Selection

In some embodiments, one or more codons of the polynucleotides of the present disclosure may be replaced with other codons encoding the native amino acid sequence to tune the expression of the SREs, through a process referred to as codon selection. Since mRNA codon, and tRNA anticodon pools tend to vary among organisms, cell types, sub cellular locations and over time, the codon selection described herein is a spatiotemporal (ST) codon selection.

In some embodiments of the disclosure, certain polynucleotide features may be codon optimized. Codon optimization refers to a process of modifying a nucleic acid sequence for enhanced expression in the host cell by replacing at least 1, 2, 3, 4, 5, 10, 15, 20, 25, 50 or more codons of the native sequence with codons that are most frequently used in the genes of that host cell while maintaining the native amino acid sequence. Codon usage may be measured using the Codon Adaptation Index (CAI) which measures the deviation of a coding polynucleotide sequence from a reference gene set. Codon usage tables are available at the Codon Usage Database (http://www.kazusa.or.jp/codon/) and the CAI can be calculated by EMBOSS CAI program (http://emboss.sourceforge.net/). Codon optimization methods are known in the art and may be useful in efforts to achieve one or more of several goals.

The stop codon of the polynucleotides of the present disclosure may be modified to include sequences and motifs to alter the expression levels of the SREs, payloads and effector modules of the present disclosure. Such sequences may be incorporated to induce stop codon read through, wherein the stop codon may specify amino acids e.g. selenocysteine or pyrrolysine. In other instances, stop codons may be skipped altogether to resume translation through an alternate open reading frame. Stop codon read through may be utilized to tune the expression of components of the effector modules at a specific ratio (e.g. as dictated by the stop codon context). Examples of preferred stop codon motifs include UGAN, UAAN, and UAGN, where N is either C or U.

Suppression of termination occurs during translation of many viral mRNAs as a means of generating a second protein with extended carboxy terminus. In retroviruses, gag and pol genes are encoded by a single mRNA and separated by an amber termination codon UAG. Translational suppression of the amber codon allows synthesis of the gag pol precursor. Translation suppression is mediated by suppressor tRNAs that can recognize termination codons and insert a specific amino acid. In some embodiments, effector modules described herein may incorporate amber termination codons. Such codons may be used in lieu of or in addition to IRES and p2A sequences in bicistronic constructs. Stop codon read through may be combined with P2A to obtain low level expression of downstream gene (e.g. IL12). In some embodiments, the amber stop codons may be combined with tRNA expression or amino-acyl tRNA synthetase for further control. In one aspect, the payload may be a regulated tRNA synthetase.

17. Subject Site

In some embodiments, the stimulus is a subject site. The subject site may a location in the subject such as, but not limited to, the blood, plasma, an organ selected from liver, kidney, brain, heart, lung, bone, and bone marrow.

18. Promoters

In some embodiments, compositions of the disclosure comprise a promoter.

As used herein a promoter is defined as a DNA sequence recognized by transcription machinery of the cell, required to initiate specific transcription of the polynucleotide sequence of the present disclosure. Vectors can comprise native or non-native promoters operably linked to the polynucleotides of the disclosure. The promoters selected may be strong, weak, constitutive, inducible, tissue specific, development stage-specific, and/or organism specific. One example of a suitable promoter is the immediate early cytomegalovirus (CMV) promoter such as, but not limited to SEQ ID NO: 6384-6386. This promoter sequence is a strong constitutive promoter sequence capable of driving high levels of expression of polynucleotide sequence that is operatively linked to it. Another example of a promoter is Elongation Growth Factor-1 Alpha (EF-1 alpha) such as, but not limited to, SEQ ID NO: 6387-6391. Other constitutive promoters may also be used, including, but not limited to simian virus 40 (SV40), mouse mammary tumor virus (MMTV), human immunodeficiency virus (HIV), long terminal repeat (LTR), promoter, an avian leukemia virus promoter, an Epstein-Barr virus immediate early promoter, a Rous sarcoma virus promoter as well as human gene promoters including, but not limited to the phosphoglycerate kinase (PGK) promoter (non-limiting examples include SEQ ID NO: 6392-6399), actin promoter, the myosin promoter, the hemoglobin promoter, the Ubiquitin C (Ubc) promoter, the human U6 small nuclear protein promoter and the creatine kinase promoter. In some instances, inducible promoters such as but not limited to metallothionine promoter, glucocorticoid promoter, a progesterone promoter, and a tetracycline promoter may be used.

In some embodiments, the optimal promoter may be selected based on its ability to achieve minimal expression of the SREs and payloads of the disclosure in the absence of the ligand and detectable expression in the presence of the ligand.

Additional promoter elements e.g. enhancers may be used to regulate the frequency of transcriptional initiation. Such regions may be located 10-100 base pairs upstream or downstream of the start site. In some instances, two or more promoter elements may be used to cooperatively or independently activate transcription.

19. Other Regulatory Features

In some embodiments, compositions of the disclosure may include optional proteasome adaptors. As used herein, the term “proteasome adaptor” refers to any nucleotide/amino acid sequence that targets the appended payload for degradation. In some aspects, the adaptors target the payload for degradation directly thereby circumventing the need for ubiquitination reactions. Proteasome adaptors may be used in conjunction with drug responsive domains to reduce the basal expression of the payload. Exemplary proteasome adaptors include the UbL domain of Rad23 or hHR23b, HPV E7 which binds to both the target protein Rb and the S4 subunit of the proteasome with high affinity, which allows direct proteasome targeting, bypassing the ubiquitination machinery; the protein gankyrin which binds to Rb and the proteasome subunit S6.

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference and understanding, and the inclusion of such definitions herein should not necessarily be construed to mean a substantial difference over what is generally understood in the art. Commonly understood definitions of molecular biology terms and/or methods and/or protocols can be found in Rieger et al., Glossary of Genetics: Classical and Molecular, 5th edition, Springer-Verlag: New York, 1991; Lewin, Genes V, Oxford University Press: New York, 1994; Sambrook et al., Molecular Cloning, A Laboratory Manual (3d ed. 2001) and Ausubel et al., Current Protocols in Molecular Biology (1994), Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929.

As appropriate, procedures involving the use of commercially available kits and/or reagents are generally carried out in accordance with manufacturer's guidance and/or protocols and/or parameters unless otherwise noted.

“Affinity” refers to the strength of binding: increased binding affinity being correlated with a lower Kd.

Adoptive cell therapy (ACT): The terms “Adoptive cell therapy” or “Adoptive cell transfer”, as used herein, refer to a cell therapy involving in the transfer of cells into a patient, wherein cells may have originated from the patient, or from another individual, and are engineered (altered) before being transferred back into the patient. The therapeutic cells may be derived from the immune system, such as effector immune cells: CD4+ T cell; CD8+ T cell, Natural Killer cell (NK cell); and B cells and tumor infiltrating lymphocytes (TILs) derived from the resected tumors. Most commonly transferred cells are autologous anti-tumor T cells after ex vivo expansion or manipulation. For example, autologous peripheral blood lymphocytes can be genetically engineered to recognize specific tumor antigens by expressing T-cell receptors (TCR) or chimeric antigen receptor (CAR).

Agent: As used herein, the term “agent” refers to a biological, pharmaceutical, or chemical compound. Non-limiting examples include simple or complex organic or inorganic molecule, a peptide, a protein, an oligonucleotide, an antibody, an antibody derivative, antibody fragment, a receptor, and soluble factor.

Agonist: the term “agonist” as used herein, refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors, e.g., agonist of a co-stimulatory receptor.

Antagonist: the term “antagonist” as used herein refers to any agent that inhibits or reduces the biological activity of the target(s) it binds.

Approximately: As used herein, the term “approximately” or “about,” as applied to one or more values of interest, refers to a value that is similar to a stated reference value. In certain embodiments, the term “approximately” or “about” refers to a range of values that fall within 25, 20, 19, 18, 17, 16, 15, 14, 13, 12, 11, 10, 9, 8, 7, 6, 5, 4, 3, 2, 1, or less in either direction (greater than or less than) of the stated reference value unless otherwise stated or otherwise evident from the context (except where such number would exceed 100 of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Autologous: the term “autologous” as used herein is meant to refer to any material derived from the same individual to which it is later to be re-introduced into the individual.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (Kd) of 10-6 M-1 or lower.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked. Constructs may can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

Cytokines: the term “cytokines”, as used herein, refers to a family of small soluble factors with pleiotropic functions that are produced by many cell types that can influence and regulate the function of the immune system.

Delivery: the term “delivery” as used herein refers to the act or manner of delivering a compound, substance, entity, moiety, cargo or payload. A “delivery agent” refers to any agent which facilitates, at least in part, the in vivo delivery of one or more substances (including, but not limited to a compound and/or composition of the present disclosure) to a cell, subject or other biological system cells.

As used herein, the phrase “derived from” as it relates to DRDs or payloads means that the DRD or payload originates at least in part from the stated parent molecule or sequence. For example, a DRD may be derived from an epitope or region of a naturally occurring protein and is modified in any of the ways taught herein to optimize DRD function.

Destabilized: As used herein, the term “destable,” “destabilize,” “destabilizing region” or “destabilizing domain” means a region or molecule that is less stable than a starting, reference, wild-type or native form of the same region or molecule.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

Engineered: As used herein, embodiments of the disclosure are “engineered” when they are designed to have a feature or property, whether structural or chemical, that varies from a starting point, wild type or native molecule.

An “exogenous” molecule is a molecule that is not normally present in a cell but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylates, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogeneous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, or other organelle, or a naturally occurring episomal nucleic acid. Additional endogenous molecules can include proteins, for example, transcription factors and enzymes.

An “episome” is a replicating nucleic acid, nucleoprotein complex or other structure comprising a nucleic acid that is not part of the chromosomal karyotype of a cell. Examples of episomes include plasmids and certain viral genomes.

“Eukaryotic” cells include, but are not limited to, fungal cells (such as yeast), plant cells, animal cells, mammalian cells and human cells (e.g., T-cells).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid.cndot.in, for example, a host cell. The expression vector can be part of a plasmid, virus, or nucleic acid fragment. Typically, the expression vector can include a nucleic acid to be transcribed operably linked to a promoter.

The term “fragment,” as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000, or more consecutive nucleotides of a nucleic acid according to the invention.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well known in the art. Similarly, methods for determining protein function are well known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

As used herein, a “functional” biological molecule is a biological entity with a structure and in a form in which it exhibits a property and/or activity by which it is characterized.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins, for example, a fusion between a DNA-binding domain (e.g., ZFP, TALE and/or meganuclease DNA-binding domains) and a nuclease (cleavage) domain (e.g., endonuclease, meganuclease, etc. and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene” refers to a polynucleotide comprising nucleotides that encode a functional molecule including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. For example, the interleukin-12 (IL-12) gene encodes the IL-12 protein. IL-12 is a heterodimer of a 35-kD subunit (p35) and a 40-kD subunit (p40) linked through a disulfide linkage to make fully functional IL-12p70. The IL-12 gene encodes both the p35 and p40 subunits.

The transcribed polynucleotide can have a sequence encoding a polypeptide, such as a functional protein, which can be translated into the encoded polypeptide when placed under the control of an appropriate regulatory region. A gene may comprise several operably linked fragments, such as a promoter, a 5′ leader sequence, a coding sequence and a 3′ nontranslated sequence, such as a polyadenylation site, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A chimeric or recombinant gene is a gene not normally found in nature, such as a gene in which, for example, the promoter is not associated in nature with part or all of the transcribed DNA region. “Expression of a gene” refers to the process wherein a gene is transcribed into an RNA and/or translated into a functional protein.

“Gene delivery” or “gene transfer” refers to methods for introduction of recombinant or foreign DNA into host cells. The transferred DNA can remain non-integrated or preferably integrates into the genome of the host cell. Gene delivery can take place for example by transduction, using viral vectors, or by transformation of cells, using known methods, such as electroporation, cell bombardment.

The term “genome” includes chromosomal as well as mitochondrial, chloroplast and viral DNA or RNA.

The term “head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head” may be abbreviated (5′)-to-(5′).

The term “tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′).

The term “head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5′)-to-(3′).

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Heterologous DNA” refers to DNA not naturally located in the cell, or in a chromosomal site of the cell. The heterologous DNA may include a gene foreign to the cell.

“Homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Immune cells: the term “an immune cell”, as used herein, refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a T γδ cell, a Tαβ cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

Immunotherapy: the term “immunotherapy” as used herein, refers to a type of treatment of a disease by the induction or restoration of the reactivity of the immune system towards the disease.

Immunotherapeutic agent: the term “immunotherapeutic agent” as used herein, refers to the treatment of disease by the induction or restoration of the reactivity of the immune system towards the disease with a biological, pharmaceutical, or chemical compound.

The term “isolated” for the purposes of the invention designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present is considered “isolated.”

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE or CRISPR/Cas system as described herein. Thus, gene inactivation may be partial or complete.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids e.g., polynucleotides). In some embodiments, wherein mutations comprise the addition and/or substitution of amino acids and/or nucleotides, such additions and/or substitutions may comprise one or more amino acid and/or nucleotide residues and may include modified amino acids and/or nucleotides. The resulting construct, molecule or sequence of a mutation, change or alteration may be referred to herein as a mutant.

“Nucleic acid,” “nucleic acid molecule,” “oligonucleotide,” “nucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA.

As used herein, an “isolated nucleic acid fragment” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

Operably linked: As used herein, the phrase “operably linked” refers to a functional connection between two or more molecules, constructs, transcripts, entities, moieties or the like.

“Operably-linked” or “functionally linked” as it refers to nucleic acid sequences and polynucleotides refers to the association of nucleic acid sequences on a single nucleic acid fragment so that the function of one is affected by the other, while the nucleic acid sequences need not necessarily be adjacent or contiguous to each other, but may have intervening sequences between them. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. A transcriptional regulatory sequence is generally operably linked in cis with a coding sequence, but need not be directly adjacent to it. For example, an enhancer is a transcriptional regulatory sequence that is operably linked to a coding sequence, even though they are not contiguous, or, a promoter is operably linked to a gene of interest if the promoter regulates or mediates transcription of the gene of interest in a cell.

Typically, it refers to the functional relationship of a transcriptional regulatory sequence to a transcribed sequence. For example, an EF-1 promoter or enhancer sequence, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to the AP-1 promoter. Polylinkers are polynucleotide sequences that comprise a series of three or more closely spaced restriction endonuclease recognition sequences.

In an association between two or more polypeptides or domains thereof to create a fusion polypeptide, the term “operably linked” means that the state or function of one polypeptide in the fusion protein is affected by the other polypeptide in the fusion protein. For example, with respect to a fusion protein comprising a DRD and a payload, the DRD and the payload are operably linked if stabilization of the DRD with a ligand results in stabilization of the payload, while destabilization of the DRD in the absence of a ligand results in destabilization of the payload. While the term operably linked may certainly include embodiments in which the DRD is adjacent or directly fused with a payload, other embodiments such as when a DRD is separated from the payload by other nucleotide sequences or peptide or polypeptide sequences is also “operably” linked to a payload, if stabilization of the DRD with a ligand results in stabilization of the payload, while destabilization of the DRD in the absence of a ligand results in destabilization of the payload.

“Open reading frame” is abbreviated ORF and refers to a length of nucleic acid sequence, either DNA, cDNA or RNA, that comprises a translation start signal or initiation codon, such as an ATG or AUG, and a termination codon and can be potentially translated into a polypeptide sequence.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally occurring amino acid.

The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Starting plasmids disclosed herein are either commercially available, publicly available on an unrestricted basis, or can be constructed from available plasmids by routine application of well-known published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

“Promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity. The promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease S1), as well as protein binding domains (consensus sequences) responsible for the binding of RNA polymerase.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

The term “purified,” as applied to biological materials does not require the material to be present in a form exhibiting absolute purity, exclusive of the presence of other compounds. It is rather a relative definition.

Payload or payload of interest (POI): the terms “payload” and “payload of interest (POI)”, as used herein, are used interchangeable. A payload of interest (POI) refers to any protein or compound whose function is to be altered. In the context of the present disclosure, the POI is a component in the immune system, including both innate and adaptive immune systems. Payloads of interest may be a protein, a fusion construct encoding a fusion protein, or non-coding gene, or variant and fragment thereof. Payload of interest or payload, may, when amino acid based, may be referred to as a protein of interest.

As used herein, the term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” refers to a variant which contains one or more amino acids which would mimic an activated sequence. For example, glutamate may serve as a mimic for phospho-threonine and/or phospho-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the pharmaceutical compositions, biocircuits, biocircuit components, effector modules including their SREs or payloads of the disclosure may comprise naturally occurring amino acids and as such may be considered to be proteins, peptides, polypeptides, or fragments thereof. Alternatively, the pharmaceutical compositions, biocircuits, biocircuit components, effector modules including their SREs or payloads may comprise both naturally and non-naturally occurring amino acids.

As used herein, the term “amino acid sequence variant” refers to molecules with some differences in their amino acid sequences as compared to a native or starting sequence. The amino acid sequence variants may possess substitutions, deletions, and/or insertions at certain positions within the amino acid sequence. As used herein, the terms “native” or “starting” when referring to sequences are relative terms referring to an original molecule against which a comparison may be made. Native or starting sequences should not be confused with wild type sequences. Native sequences or molecules may represent the wild-type (that sequence found in nature) but do not have to be identical to the wild-type sequence.

Ordinarily, variants will possess at least about 70% homology to a native sequence, and preferably, they will be at least about 80%, more preferably at least about 90% homologous to a native sequence.

As used herein, the term “homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

As used herein, the term “homolog” as it applies to amino acid sequences is meant the corresponding sequence of other species having substantial identity to a second sequence of a second species.

As used herein, the term “analog” is meant to include polypeptide variants which differ by one or more amino acid alterations, e.g., substitutions, additions or deletions of amino acid residues that still maintain the properties of the parent polypeptide.

As used herein, the term “derivative” is used synonymously with the term “variant” and refers to a molecule that has been modified or changed in any way relative to a reference molecule or starting molecule.

Pharmaceutically acceptable excipients: the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N, N′-dimethylformamide (DMF), N, N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e, the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide. In any of the methods described herein, additional pairs of gene editing nucleases can be used for additional double-stranded cleavage of additional target sites within the cell.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), beta.-galactosidase (LacZ), .beta.-glucuronidase (Gus), and the like. Selectable marker genes may also be considered reporter genes.

The term “response element” refers to one or more cis-acting DNA elements which confer responsiveness on a promoter mediated through interaction with the DNA-binding domains of a transcription factor. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds, in the presence or absence of a ligand, to the DNA sequence of a response element to initiate or suppress transcription of downstream gene(s) under the regulation of this response element.

The term “sequence” refers to a nucleotide sequence of any length, which can be DNA or RNA; can be linear, circular or branched and can be either single-stranded or double stranded.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

Stable: As used herein “stable” refers to a compound or entity that is sufficiently robust to survive isolation to a useful degree of purity from a reaction mixture, and preferably capable of formulation into an efficacious therapeutic agent.

As used herein, the term “stabilize”, “stabilized,” “stabilized region” means to make or become stable. In some embodiments, stability is measured relative to an absolute value. In some embodiments, stability is measured relative to a secondary status or state or to a reference compound or entity.

As used herein, the term “standard CAR” refers to the standard design of a chimeric antigen receptor. The components of a CAR fusion protein including the extracellular scFv fragment, transmembrane domain and one or more intracellular domains are linearly constructed as a single fusion protein.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any patient or subject (e.g. mammalian) to which the cells or stem cells of the invention can be administered.

A T cell is an immune cell that produces T cell receptors (TCRs). T cells can be naïve (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cell and effector memory T cells (TEM, decreased expression of CD62L, CCR7, CD28, CD45RA, and increased expression of CD127 as compared to naïve T cells or TCM). Effector T cells (TE) refers to antigen-experienced CD8+ cytotoxic T lymphocytes that have decreased expression of CD62L, CCR7, CD28, and are positive for granzyme and perforin as compared to TCM. Other exemplary T cells include regulatory T cells, such as CD4+CD25+(Foxp3+) regulatory T cells and Treg17 cells, as well as Tr1, Th3, CD8+CD28−, and Qa-1 restricted T cells.

T cell receptor: T cell receptor (TCR) refers to an immunoglobulin superfamily member having a variable antigen binding domain, a constant domain, a transmembrane region, and a short cytoplasmic tail, which is capable of specifically binding to an antigen peptide bound to a MEW receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, β-chain) contains two immunoglobulin domains, a variable domain (e.g., α-chain variable domain or Vα, β-chain variable domain or Vβ) at the N terminus, and one constant domain (e.g., α-chain constant domain or Cα and β-chain constant domain or Cβ) adjacent to the cell membrane. Similar to immunoglobulin, the variable domains contain complementary determining regions (CDRs) separated by framework regions (FRs). A TCR is usually associated with the CD3 complex to form a TCR complex. As used herein, the term “TCR complex” refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCR chain. A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Treatment or treating: As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

Tune: As used herein, the term “tune” means to adjust, balance or adapt one thing in response to a stimulus or toward a particular outcome. In one non-limiting example, the DRDs of the present disclosure adjust, balance or adapt the function or structure of compositions to which they are appended, attached or associated with in response to particular stimuli and/or environments.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

A “target site” or “target sequence” is a nucleic acid sequence that defines a portion of a nucleic acid to which a binding molecule will bind, provided sufficient conditions for binding exist. An “intended” target site is one that the DNA-binding molecule is designed and/or selected to bind to.

Transcription refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest.

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Transgene” refers to a gene that has been introduced into a host cell. The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell.

“Transduction” refers to the delivery of a nucleic acid molecule into a recipient host cell, such as by a gene delivery vector, such as a lentiviral vector, or a rAAV. For example, transduction of a target cell by a rAAV virion leads to transfer of the rAAV vector contained in that virion into the transduced cell. “Host cell” or “target cell” refers to the cell into which the nucleic acid delivery takes place.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

The term “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

“Transcriptional and translational control sequences” refer to DNA regulatory sequences, such as promoters, enhancers, terminators, and the like, that provide for the expression of a coding sequence in a host cell. In eukaryotic cells, polyadenylation signals are control sequences.

A “variant” of a molecule such as a modulator of AP-1 is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

A “cloning vector” refers to a “replicon,” which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

The term “expression vector” refers to a vector, plasmid or vehicle designed to enable the expression of an inserted nucleic acid sequence. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TP1, alkaline phosphatase promoters (useful for expression in Saccharomyces); AOX1 promoter (useful for expression in Pichia); beta-lactamase, lac, ara, tet, trp, 1PL, 1PR, T7, tac, and trc promoters (useful for expression in Escherichia coli); light regulated-, seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus 1E1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, .alpha.-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell .alpha.-actin, and the like. In addition, these expression sequences may be modified by addition of enhancer or regulatory sequences and the like.

Vectors may be introduced into the desired host cells, by methods known in the art, e.g., transfection, electroporation, microinjection, transduction, cell fusion, DEAF dextran, calcium phosphate precipitation, lipofection (lysosome fusion), use of a gene gun, or a DNA vector transporter (see, e.g., Wu et al., J. Biol. Chem. 267:963 (1992); Wu et al., J. Biol. Chem. 263:14621 (1988); and Hartmut et al., Canadian Patent Application No. 2,012,311).

Viral vectors, and particularly lentiviral and retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

Several methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as lentiviruses, vaccinia virus or AAV, or adenovirus; insect viruses such as baculovirus; yeast vectors; bacteriophage vectors (e.g., lambda), and plasmid and cosmid DNA vectors, to name but a few. A vector of the invention may also be administered to a subject by any route of administration, including, but not limited to, intramuscular administration.

A polynucleotide according to the disclosure can also be introduced in vivo by lipofection. There has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker. The use of cationic lipids may promote encapsulation of negatively charged nucleic acids, and also promote fusion with negatively charged cell membranes. Particularly useful lipid compounds and compositions for transfer of nucleic acids are described in WO 95/18863, WO 96/17823 and U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting. Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., WO 95/21931), peptides derived from DNA binding proteins (e.g., WO 96/25508), or a cationic polymer (e.g., WO 95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used.

In addition, the recombinant vector comprising a polynucleotide according to the invention may include one or more origins for replication in the cellular hosts in which their amplification or their expression is sought, markers or selectable markers.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein, the term “insertional variants” when referring to proteins are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid.

As used herein, the term “deletional variants” when referring to proteins, are those with one or more amino acids in the native or starting amino acid sequence removed. Ordinarily, deletional variants will have one or more amino acids deleted in a particular region of the molecule.

As used herein, the term “derivatives,” as referred to herein includes variants of a native or starting protein comprising one or more modifications with organic proteinaceous or non-proteinaceous derivatizing agents, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Features of the proteins of the present disclosure include surface manifestations, local conformational shape, folds, loops, half-loops, domains, half-domains, sites, termini or any combination thereof. As used herein, the term “features” when referring to proteins are defined as distinct amino acid sequence-based components of a molecule.

As used herein, the term “surface manifestation” when referring to proteins refers to a polypeptide based component of a protein appearing on an outermost surface.

As used herein, the term “local conformational shape” when referring to proteins refers to a polypeptide based structural manifestation of a protein which is located within a definable space of the protein.

As used herein, the term “fold,” when referring to proteins, refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein, the term “turn” as it relates to protein conformation, refers to a bend which alters the direction of the backbone of a peptide or polypeptide and may involve one, two, three or more amino acid residues.

As used herein, the term “loop,” when referring to proteins, refers to a structural feature of a peptide or polypeptide which reverses the direction of the backbone of a peptide or polypeptide and comprises four or more amino acid residues. Oliva et al. have identified at least 5 classes of protein loops (Oliva, B. et al., An automated classification of the structure of protein loops. J Mol Biol. 1997. 266(4):814-30.)

As used herein, the term “half-loop,” when referring to proteins, refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein, the term “domain,” when referring to proteins, refers to a motif of a polypeptide having one or more identifiable structural or functional characteristics or properties (e.g., binding capacity, serving as a site for protein-protein interactions.)

As used herein, the term “half-domain,” when referring to proteins, refers to a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, the terms “site,” as it pertains to amino acid based embodiments is used synonymously with “amino acid residue” and “amino acid side chain”. A site represents a position within a peptide or polypeptide that may be modified, manipulated, altered, derivatized or varied within the polypeptide based molecules of the present disclosure.

As used herein, the terms “termini” or “terminus,” when referring to proteins refers to an extremity of a peptide or polypeptide. Such extremity is not limited only to the first or final site of the peptide or polypeptide but may include additional amino acids in the terminal regions. The polypeptide based molecules of the present disclosure may be characterized as having both an N-terminus (terminated by an amino acid with a free amino group (NH2)) and a C-terminus (terminated by an amino acid with a free carboxyl group (COOH)). “Wild-type” refers to a virus or organism found in nature without any known mutation.

Zinc finger and TALE DNA binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,586,526; 6,140,081; 6,453,242; 6,534,261 and 8,586,526; see also WO 98/53058; WO 98/53059; WO 98/53060; WO 02/016536 and WO 03/016496.

The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

As an example of disease-specific promoters, useful promoters for treating cancer include the promoters of oncogenes, including promoters for treating anemia. Examples of classes of oncogenes include, but are not limited to, growth factors, growth factor receptors, protein kinases, programmed cell death regulators and transcription factors.

Examples of promoter sequences and other regulatory elements (e.g., enhancers) that are known in the art and are useful as therapeutic switch promoters in the present invention are disclosed in U.S. Pat. No. 9,402,919, Ser. No. 14/001,943, filed on Mar. 2, 2012.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure. The present disclosure is further illustrated by the following nonlimiting examples.

### EXAMPLES

### Example 1. Generation of Novel Ligand Responsive SREs or DDs by Mutagenesis Screening

Methods for making DRDs useful in the compositions and methods of the present disclosure are described and exemplified in the following published applications, WO 2018/161000; WO 2018/231759; WO 2019/241315; WO 2018/160993; WO 2018/237323; and WO 2018/161038, the disclosures of the aforementioned applications relating to the methods for identifying, screening and isolating the exemplified DRDs are incorporated herein by reference in their entireties.

### Example 2. In Vitro Regulation of Membrane Bound CD40L Transduced Jurkat Cells and Primary T Cells

50,000 Jurkat cells were transduced with lentiviruses corresponding to the constructs. OT-001661, OT-001685, OT-001662, or OT-001666 and cultured for 48h. Cells were split and incubated with ligands for 24h before analysis by flow cytometry.

Jurkat cells were treated with one of the following: 1 μM Shield-1, 50 μM TMP, 1 μM Bazedoxifene (BZD) or vehicle control. Surface expression of CD40L was measured using FACS and the results are show in Table 9. All values were normalized to mode.

TABLE 9
Surface expression of CD40L

| | | | | Geometric |
|---|---|---|---|---|
| | | | CD40L | Mean of |
| | | | positive | CD40L |
| Construct | | | percent of | fluorescent |
| Name | DRD | Ligand | live cells | intensity |
| Untransduced | — | — | 0.78 | 22.9 |
| OT-001661 | — | — | 99.4 | 10075 |
| OT-001685 | FKBP (M1del, | Vehicle | 0.056 | 37.1 |
| | F37V, L107P) | (Ethanol 0.2%) | | |
| | | 1 μM Shield-1 | 97.2 | 1996 |
| OT-001662 | ecDHFR | Vehicle | 0.023 | 27.9 |
| | (M1del, R12Y, | (DMSO 0.5%) | | |
| | Y100I) | 50 μM TMP | 98.4 | 6068 |
| OT-001666 | ER (305-549 of | Vehicle | 0.024 | 21.5 |
| | WT, L384M, | (DMSO | | |
| | N413D, M421G, | 0.01%) | | |
| | G521R, Y537S) | 1 μM | 38.4 | 421 |
| | | Bazedoxifene. | | |

Very low baseline activity was observed with all constructs tested. In the presence of their corresponding ligand, the FKBP and ecDHFR DRD based constructs showed strong ligand dependent regulation. Modest ligand dependent regulation was observed with the ER DRD based construct. As expected, Jurkat cells transduced with the constitutively expressed construct OT-001661 showed CD40L expression at levels higher than untransduced cells.

Jurkat cells transduced with OT-001661, OT-001685, OT-001662, OT-001666, or OT-001667 were co-cultured with HEK-Blue™ CD40 cells (Invivogen, San Diego, Calif.). Cocultures were treated with ligands (1 μM Shield-1, 50 μM TMP, 1 μM Bazedoxifene (BZD) or vehicle control) for 24 hours and the secreted embryonic alkaline phosphatase (SEAP) reporter levels were measured in the media. The pg/mL levels of soluble CD40L in ligand treated samples is shown in Table 10. All vehicle treated samples had background SEAP activity. Recombinant soluble CD40L was used as the standard in the experiments.

TABLE 10
CD40L levels in the presence of ligand

| | Soluble CD40L |
|---|---|
| Construct Name | (pg/ml) |
| OT-001661 | 9341.40 |
| OT-001685 | 4833.64 |
| OT-001662 | 8657.75 |
| OT-001666 | 1824.7 |
| OT-001667 | 2565.91 |

The detection of soluble CD40L in the presence of ligand and its virtual absence in the vehicle controls suggests that all constructs are functional in Jurkat cells and are associated with detectable levels of CD40L.

Regulation of ecDHFR (R12Y, Y100I) DRD based construct OT-001662 and ER (N413D) DRD based construct OT-001666 were tested in CD8 positive T cells. For OT-001662, expressing cells, ligand was added 6 days after viral transduction at a dose of 50 μM TMP. OT-001666 expressing cells were treated with 0.5 μM Bazedoxifene or vehicle control, 5 days after transduction. Approximately 15-30k cells were analyzed per sample by FACS. All samples were compared to empty vector control as well as the constitutively expressed construct, OT-001661. Table 11 provides the mean fluorescence intensities (MFIs) obtained with each sample.

TABLE 11
CD40L MFIs

| Construct | Vehicle | +Ligand |
|---|---|---|
| Empty | 283 | — |
| Vector | | |
| OT-001662 | 29.0 | 283 |
| OT-001666 | 35.4 | 33.1 |

Very low baseline activity was observed with both the constructs tested. Expression of CD40L in the ecDHFR DRD based construct OT-001662, in the presence of ligand was higher than the ER DRD based construct, OT-001666.

Ligand dose response curve experiments were performed with increasing doses of ligand using OT-001662 and OT-001666 constructs in both CD4+ as well as CD8+ T cells.

The experiments were conducted using three different volumes of virus for transduction i.e. 0.5, 2 and 10 μL. The results are shown in Table 12 and Table 13.

TABLE 12
Dose response with OT-001662

| TMP | CD4+ | | | CD8+ | | |
|---|---|---|---|---|---|---|
| (nM) | 0.5 μL | 2 μL | 10 μL | 0.5 μL | 2 μL | 10 μL |
| 50000 | 68.9 | 76.8 | 78.8 | 25.2 | 51 | 59.4 |
| 5000 | 67 | 76.8 | 79.1 | 9.21 | 40.6 | 47.4 |
| 500 | 48.2 | 68.4 | 58.5 | 0.66 | 6.84 | 5.53 |
| 50 | 35.7 | 37.4 | 30.7 | 0.19 | 0.64 | 1.07 |
| 5 | 33.9 | 27.6 | 24.6 | 0.12 | 0.3 | 1.02 |
| 0.5 | 32 | 24.4 | 24.2 | 0.17 | 0.34 | 1.08 |
| 0.05 | 31.6 | 25.1 | 23.8 | 0.16 | 0.2 | 0.95 |

TABLE 13
Dose response with OT-001666

| BZD | CD4+ | | CD8+ | |
|---|---|---|---|---|
| (nM) | 2 μL | 10 μL | 2 μL | 10 μL |
| 500 | 71.8 | 76.5 | 60 | 68.5 |
| 50 | 36 | 34 | 3.32 | 17.7 |
| 5 | 11.1 | 6.42 | 0.71 | 0.23 |
| 0.5 | 9.25 | 4.86 | 0.52 | 0.2 |
| 0.05 | 8.57 | 4.16 | 0.69 | 0.5 |

The results in Table 12 and Table 13 show that dose responsive ligand mediated stabilization is evident in the CD4+ T cells at lower concentrations of ligand for both constructs. Additionally, higher levels of % CD40L positive cells at the higher doses were obtained with the CD4+ T cells. These results may be influenced by the expression of endogenous CD40L in CD4+ T cells. Comparatively, CD8+ T cells showed lower basal expression and lower levels of % CD40 positive cells at higher ligand doses. Since CD8+ cells do not express CD40L from their endogenous locus, they may likely exhibit lower basal expression levels.

OT-001663 and OT-001664 demonstrated ligand dependent stabilization at the protein level as measured via western blot.

### Example 3. In Vitro Regulation of Soluble CD40L Transduced Jurkat Cells and Primary T Cells

Soluble CD40L (sCD40L) constructs OT-001672, OT-001686, OT-001673, OT-001674, OT-001677, and OT-001684 were transiently transfected into HEK293T cells. Cells were treated with one of the following ligands 1 μM Shield-1, 50 μM TMP, 1 μM Bazedoxifene (BZD) or vehicle control.

The pg/mL levels of soluble CD40L were measured by ELISA (Table 14). Recombinant soluble CD40L was used as the standard in the experiments.

TABLE 14
Soluble CD40L levels

| | Soluble CD40L | | |
|---|---|---|---|
| | (μg/ml) | | Stabilization |
| Name | Vehicle | +Ligand | Ratio |
| OT-001686 | — | 53475 | — |
| OT-001672 | 9583.76 | 31686.1 | 3.3 |
| OT-001673 | 1 | 48196.8 | 48196.8 |
| OT-001674 | 215.96 | 1645.44 | 7.6 |
| OT-001684 | 3 | 3 | 1.0 |

As shown in Table 14, ligand dependent regulation of soluble CD40L levels was observed with OT-001686, OT-001673, and OT-001674. In an independent experiment with similar setup, OT-001677 showed a stabilization ratio of 7.2 with very low basal expression in the absence of ligand. HEK-Blue CD40 assay performed with the sCD40L constructs shows that all constructs are functional. Constructs OT-001672, OT-001686, and OT-001673 showed higher functionality than the other constructs tested. No detectable activity was observed with the cells treated with the corresponding vehicle controls.

### Example 4. Engineering CD40L to Reduce Shedding

Sheddases e.g. ADAM10/17 present in the tumor microenvironment can cleave CD40L thereby preventing the successful activation of CD40 by CD40L. Analysis of the sequence of CD40L reveals an ADAM10/17 proteolytic cleavage site. OT-001669 which has a deletion of amino acids 1-13 of CD40L was designed to reduce internalization and OT-001668 which has a deletion of amino acids 110-116 of CD40L was designed to remove the ADAM10/17 sites. Jurkat cells stably expressing the full length construct OT-001661, OT-001669, or OT-001668 were cultured for 6 hours with 1.25 μg/ml recombinant human (h) or mouse (m) CD40-Fc receptor or left untreated and tested using an ELISA assay (Table 15).

TABLE 15
CD40L levels in supernatant

| | | hCD40- | mCD40- |
|---|---|---|---|
| Construct | Untreated | Fc | Fc |
| OT-001661 | 1382.06 | 3599.72 | 2589.21 |
| OT-001669 | 2137.69 | 8444.30 | 4335.76 |
| OT-001668 | 10 | 10 | 10 |

As seen in Table 15, shedding was observed even in the absence of treatment with CD40-Fc indicating that there is some constitutive shedding. Human CD40-Fc appeared to enhance the shedding in OT-001669 expressing cells, however no shedding was observed with OT-001668, which lacks the ADAM10/17 site. Murine CD40-Fc also showed activity with human CD40L suggesting inter-species cross reactivity.

### Example 5. Functional Analysis of Regulated CD40L

In order to engage dendritic cells in an immune response, dendritic cells must be converted to a functional state by an antigen-specific CD4+ helper T cell. Activation of the dendritic cells may be followed by the activation of CD8+ T cells by the dendritic cells, a process referred to as dendritic cell licensing. Engagement of the CD40L expressed on the CD4+ cells with the CD40 on the dendritic cells can result in (i) dendritic cell stimulation-measured by expression of co-stimulatory and MHC molecules (ii) proinflammatory cytokine release (e.g. IL12, TNFα and INFγ) (iii) epitope spreading. To measure the effect of CD40L expression that is tuned by the biocircuits of the present disclosure, CD4+ T cells or Jurkat cells expressing any of the CD40L constructs described herein were co-cultured with dendritic cells.

Dendritic cell function in response to co-culture with T cells expressing CD40L was evaluated in the presence of ligand or corresponding vehicle control. Monocyte derived dendritic cells (mDC) (CD14+) were isolated from fresh blood and cultured for 5 days with IL-4 (7.5 ng/mL) and GM-CSF (20 ng/mL). The mDCs were frozen down after the 5 day culture. T cells were transduced with OT-001662 and expanded for 9 days before being frozen down. mDCs and T cells were thawed and co-cultured 1:10 DC: T cell ratio (i.e. 5×10⁴ DCs: 5×10⁵ T cells). mDC and T cells were co-cultured for 2 days before cells were collected for flow cytometry and supernatants were collected for cytokine analysis. For evaluation of regulated constructs, ligand (various doses of TMP) was added at the time initiating the co-culture and was kept in the co-culture for 2 days. Cytokines were analyzed by MSD after a single freeze thaw cycle. All cells were cultured in complete RPMI with 10% FBS during the course of the experiment. The percentage of cells in the mDC-T co-culture expressing CD40L and their MFI are shown in Table 16. SR indicates stabilization ratio.

TABLE 16
CD40L levels in T cells with mDC co-culture

| | | | | | | CD8+ | | | |
|---|---|---|---|---|---|---|---|---|---|
| | | CD4+ | | | | | | | SR |
| | | | SR | | SR | | SR | | (based |
| | | % | (based | CD40L | (based | % | (based | CD40L | on |
| Construct | Ligand | CD40L+ | on %) | MFI | on MFI | CD40L+ | on %) | MFI | MFI |
| Empty | DMSO | 11.5 | — | 125 | | 1.1 | | 62.5 | |
| Vector | TMP | 11.1 | — | 122 | | 1.02 | | 61.2 | |
| OT-001661 | DMSO | 68.1 | — | 579 | | 62.2 | | 495 | |
| | TMP | 68.3 | — | 580 | | 62.3 | | 499 | |
| OT-001662 | TMP | 84.1 | 22.67 | 1379 | 21.61 | 74.9 | 159.36 | 951 | 16.45 |
| | (10 μM) | | | | | | | | |
| | TMP | 78 | 21.02 | 691 | 10.83 | 57.8 | 122.98 | 419 | 7.25 |
| | (2 μM) | | | | | | | | |
| | TMP (0.5 | 3.34 | 0.90 | 61.1 | 0.96 | 0.57 | 1.21 | 54.5 | 0.94 |
| | μM) | | | | | | | | |
| | TMP (0.1 | 4.32 | 1.16 | 69.8 | 1.09 | 0.69 | 1.47 | 61.5 | 1.06 |
| | μM) | | | | | | | | |
| | DMSO | 3.71 | — | 63.8 | — | 0.47 | | 57.8 | |

Ligand dependent regulation of CD40L was evident in both the CD4 and the CD8+ cells, especially at higher doses of TMP in the cocultures comprising OT-001662 as evidenced by the stabilization ratios. Compared to CD8+ cells, higher MFIs were observed in CD4+ cells but only at higher doses of TMP. Regulated CD40L also induced changes in cytokine production the DC co-culture assay (See Table 17).

TABLE 17
Cytokine Production (pg/ml)

| Construct | Ligand | IFNg | IL12 | TNFa |
|---|---|---|---|---|
| Empty Vector | DMSO | 6647.38 | 0.4868 | 156.7256 |
| | TMP | 8043.85 | 0.6782 | 153.6723 |
| OT-001661 | DMSO | 12973 | 58.935 | 456.3242 |
| | TMP | 10939.9 | 86.229 | 440.0804 |
| OT-001662 | TMP (10 μM) | 13539.7 | 59.254 | 251.2844 |
| | TMP (2 μM) | 16375.2 | 19.372 | 248.7836 |
| | TMP (0.5 μM) | 3587.14 | — | 83.86263 |
| | TMP (0.1 μM) | 7432.13 | 0.0705 | 130.9627 |
| | DMSO | 3791.2 | 0.2535 | 82.2712 |

The levels of IFNγ obtained with 10 μM TMP in OT-001662 was comparable to the levels observed with the constitutive construct OT-001661, while the lower doses of TMP showed a dose responsive increase in IFNγ. TMP dose responsive increases in IL12 and TNFα were also observed.

### Example 6. PDE5 Regulated CD40L

Construct OT-001892 with GGSGGGSGGGSG linker was tested in HEK293T cells. 1 μg DNA corresponding to OT-001892 was transfected into 300,000 cells. 24 hours after transfection cells were treated with ligand (1 μM Vardenafil) or vehicle control for additional 24 hours. Surface expression of CD40L was analyzed by FACS. 30% of the OT-001892 transfected HEK293T cells treated with 1 μM Vardenafil were positive for CD40L whereas only 9% of the OT-001892 transfected HEK293T treated with vehicle control were positive for CD40L expression. Thus, ligand dependent increase in the percentage of CD40L positive cells was observed.

Construct OT-001892 expression was also measured in the context of transduced Jurkat and T cells. Jurkat cells were plated at 100,00 per dish and transduced with 10 μL of lentivirus corresponding to OT-001892. 24 hours after transfection cells were treated with ligand (1 μM Vardenafil) or vehicle control for additional 24 hours. Surface expression of CD40L was analyzed by FACS. 37.6% of the OT-001892 transduced Jurkat cells treated with 1 μM Vardenafil were positive for CD40L whereas only 2.7% of the OT-001892 transduced Jurkat cells treated with vehicle control were positive for CD40L expression. A median fluorescence intensity of 233 was observed in the 1 μM Vardenafil treated cells as compared to an MFI of −187 in the vehicle control treated group.

T cells were transduced with 10 μL of lentivirus corresponding to OT-001892. 4 days after transduction, cells were treated with increasing concentrations of ligand (Vardenafil) or vehicle control for 24 hours. Surface expression of CD40L in T cells as well as the CD4+ and CD8+ subsets was analyzed by FACS. The results are shown in Table 18. The stabilization ratio (SR) may be defined as the ratio of expression, CD40L in response to vardenafil to the expression of CD40L in the absence of vardenafil.

TABLE 18
CD40L levels in PDE5 regulated CD40L construct

| | All Cells | | CD4+ Cells | | CD8+ Cells | |
|---|---|---|---|---|---|---|
| Vardenafil | MFI | SR | MFI | SR | MFI | SR |
| DMSO | 209 | | 227 | | 144 | |
| 0.0001 μM | 274 | 1.31 | 313 | 1.38 | 166 | 1.15 |
| 0.001 μM | 260 | 1.24 | 291 | 1.28 | 166 | 1.15 |
| 0.01 μM | 265 | 1.27 | 304 | 1.34 | 162 | 1.13 |
| 0.1 μM | 311 | 1.49 | 359 | 1.58 | 171 | 1.19 |
| 1 μM | 405 | 1.94 | 482 | 2.12 | 186 | 1.29 |
| 10 μM | 747 | 3.57 | 930 | 4.10 | 340 | 2.36 |
| 100 μM | 1168 | 5.59 | 1367 | 6.02 | 620 | 4.31 |

As shown in Table 18, the higher stabilization ratios were obtained in the CD4+ population compared to the CD8+ cells indicating higher expression levels. A ligand dose dependent response was observed in all the cell populations measured and as little as 0.0001 μM vardenafil resulted in a Stabilization ratio greater than 1. Comparison of the MFIs obtained with the empty vector and DMSO treated OT-001892 transduced CD4+ cells showed that the CD40L expression is lower in the DMSO treated cells compared to empty vector expressing cells. The destabilization of the endogenous CD40L in DMSO treated cells may result in the observed reduction in CD40L expression in DMSO treated cells.

### Example 7. Efficacy of CD40L Co-Expression with CD19 CAR in Tumor Model

In Vitro Study

In order to determine if transduced T cells co-express CD19-CAR and CD40L, activated T cells were lentivirally transduced with empty vectors (EV), constitutive CD40L (OT-001661), CD19 CAR (OT-001407), and CD40L with CD19 CAR (0T-001605), grown for 2 days and then the CD40L and CD19 surface levels were measured where increased expression was seen for the CD19 CAR and CD40L and CD19 CAR constructs in T cells. The results for CD4+ and CD8+ cells and total cells are shown below.

TABLE 19
Percent of Cells co-expressing CD40L and CD19 CAR

| Construct | CD4+ | CD8+ | All Cells |
|---|---|---|---|
| Empty Vector | 0% | 0% | 0% |
| OT-001661 | 0% | 0% | 0% |
| OT-001407 | 19.9% | 1.2% | 15.7% |
| (0.1 uL) | | | |
| OT-001407 | 13.8% | 0.6% | 10.9% |
| (0.05 uL) | | | |
| OT-001605 | 20.9% | 10.9% | 18.9% |

In Vivo Study

Increased expression of CD40L on CD19 positive CART cells may increase the efficacy of CAR+ T cells in vivo. To test this, 8-week old mice were injected with Nalm6-luc (1 million per mouse). On day 6, Nalm6 tumor burden was measured and mice were grouped to ensure that all groups had similar tumor sizes. On day 7, T cells transduced with constructs were injected into mice and the groups shown in Table 20 were established. Body weight and Bioluminescence Intensity (BLI) were measured twice a week. Average BLI values are shown in Table 21. Blood and plasma were also collected for cytokine analysis, CD40L and IL12.

TABLE 20
Tumor study cohorts

| | | CAR + | |
|---|---|---|---|
| | No. | T cell | |
| | of | # | |
| Group | Mice | ×106 | Construct |
| 1 | 8 | — | Empty Vector |
| 2 | 8 | — | CD40L only (OT-001661) |
| 3 | 4 | 3 | CD19CAR only (OT-001407) |
| 4 | 8 | 1 | CD19CAR only (OT-001407) |
| 5 | 8 | 0.3 | CD19CAR only (OT-001407) |
| 6 | 5 | 3 | CD19CAR and CD40L (OT-001605) |
| 7 | | 1 | CD19CAR and CD40L (OT-001605) |
| 8 | 8 | 0.3 | CD19CAR and CD40L (OT-001605) |
| 10 | 8 | 1 | Control CD19CAR only (OT-001662) |

TABLE 21
Average BLI values

| Construct | | | | | | | | |
|---|---|---|---|---|---|---|---|---|
| (below)/Day | | | | | | | | |
| (Across) | 6 | 13 | 20 | 25 | 28 | 32 | 36 | 39 |
| Empty | 2.04E+06 | 1.97E+08 | 2.21E+09 | 5.35E+09 | 1.41E+10 | — | — | — |
| Vector | | | | | | | | |
| OT-001661 | 2.04E+06 | 2.69E+08 | 2.52E+09 | 5.99E+09 | 1.04E+10 | — | — | — |
| 3M CAR + | 2.02E+06 | 4.24E+06 | 7.47E+05 | 8.06E+05 | 8.39E+05 | 8.87E+05 | 1.35E+06 | 4.72E+06 |
| OT- | | | | | | | | |
| 001407(n = 4) | | | | | | | | |
| 1M CAR + | 2.02E+06 | 4.88E+07 | 3.99E+07 | 1.04E+08 | 3.15E+08 | 5.87E+09 | 3.33E+09 | 9.80E+09 |
| OT-001407 | | | | | | | | |
| (n = 5 of 8) | | | | | | | | |
| 0.3M CAR + | 2.01E+06 | 1.09E+08 | 5.34E+08 | 1.25E+09 | 1.34E+09 | 2.20E+09 | 7.92E+09 | 7.26E+09 |
| OT- | | | | | | | | |
| 001407(n = 4 | | | | | | | | |
| of 8) | | | | | | | | |
| 3M CAR + | 1.98E+06 | 3.09E+07 | 1.00E+06 | 7.54E+05 | 7.39E+05 | 8.05E+05 | 7.23E+05 | 6.97E+05 |
| OT-001605 | | | | | | | | |
| (n = 5) | | | | | | | | |
| 1M CAR + | 2.02E+06 | 2.82E+07 | 8.19E+05 | 8.93E+05 | 1.11E+06 | 1.07E+06 | 9.11E+05 | 7.12E+05 |
| OT-001605 | | | | | | | | |
| 0.3M CAR + | 2.03E+06 | 1.27E+08 | 2.44E+08 | 2.27E+07 | 5.81E+06 | 2.91E+06 | 2.52E+06 | 2.66E+06 |
| OT-001605 | | | | | | | | |
| 1M CAR + | 2.05E+06 | 6.32E+06 | 1.26E+06 | 3.46E+06 | 6.89E+06 | 6.14E+07 | 4.49E+08 | 3.53E+09 |
| OT-001607 | | | | | | | | |
| (IRES) | | | | | | | | |
| 1M CAR + | 2.06E+06 | 2.12E+08 | 1.23E+09 | 4.55E+09 | 8.13E+09 | 3.62E+09 | 8.85E+09 | 3.50E+09 |
| OT-001607: | | | | | | | | |
| Control | | | | | | | | |
| Batch (n = 2 | | | | | | | | |
| of 8) | | | | | | | | |

As shown in Table 21, BLI values decreased when CD19CAR-2A-CD40L T cells were injected into NALM6 mice indicating reduction in tumor burden. While CD19CAR only cells showed some reduction in BLI, it was not as significant as significant as the CD19CAR-2A-CD40L group.

Human cells were analyzed by FACS (hCD45+) from the blood 14 days after T-cell infusion, which equals 21 days after tumor implant. The results are shown in Table 22 where M indicates million.

TABLE 22
Percentage hCD45+ cells

| Construct | Individual values | | | | Average |
|---|---|---|---|---|---|
| Empty vector | 0.027 | 0.57 | 0.27 | 0.062 | 0.232 |
| OT-001661 | 0.00249 | 0.00331 | 0.00198 | 0 | 0.002 |
| OT-001407 (3M) | 0.1 | 1.06 | 0.72 | 1.65 | 0.883 |
| OT-001407 (1M) | 0.4 | 0.081 | 0.12 | 0.028 | 0.157 |
| OT-001407 (0.3M) | 0.012 | 0.019 | 0.00803 | 0.00702 | 0.012 |
| OT-001605 (3M) | 26.9 | 12.8 | 13.2 | 14.9 | 16.950 |
| OT-001605 (1M) | 37 | 5.29 | 27.8 | 4.23 | 18.580 |
| OT-001605 (0.3M) | 0.032 | 8.35 | 2.46 | 6.23 | 4.268 |
| OT-001607 (1M) | 0.45 | 0.86 | 0.67 | 0.46 | 0.610 |
| OT-001607 | 0.014 | 0.011 | 0.5 | 0.012 | 0.134 |
| (Control) (1M) | | | | | |

As shown in Table 22, OT-001605 (1M) showed the greatest expansion of T cells with all 3 cell number groups. These results show that CD40L expression increases CAR dependent T-cell expansion in vivo.

### Example 8. CD40L Kinetics in Jurkat Cells

Kinetics were tested using stably transduced Jurkat cells expressing OT-001662. To study the kinetics of turning on the expression of CD40L, cells were treated with TMP (5 or 50 μM) and fixed for FACS after: 0, 3, 6, 9, 24, 48 hours. At the end of the experiment all fixed were stained and analyzed via FACS in parallel. To study the kinetics of turning off expression, expression was first induced in Jurkat cells with 50 μM TMP. The cells were then washed to remove TMP and fixed after 0, 3, 6, 9, 24 hours, stained and analyzed via FACS in parallel. Table 23 and Table 24 shown the kinetics of turning expression on and off respectively.

TABLE 23
On kinetics

| | TMP | 5 μM | |
|---|---|---|---|
| Hours | (50 μM) | TMP | Vehicle |
| 0 | 743 | 743 | 740 |
| 3 | 890 | 886 | 765 |
| 6 | 1180 | 1096 | 783 |
| 9 | 1466 | 1392 | 772 |
| 24 | 3952 | 2435 | 778 |

Off kinetics

| | TMP | |
|---|---|---|
| Hours | (50 μM) | Vehicle |
| 0 | 3952 | 778 |
| 3 | 1631 | 829 |
| 6 | 1620 | 823 |
| 9 | 1037 | 823 |
| 24 | 1287 | 923 |

As shown in Table 23 and Table 24, surface expression gradually increases over 24 hours possibly due to the need for CD40L to trimerize for trafficking. However, the off kinetics appear fast indicating that half-life of surface CD40L is short. Alternatively, it also suggests that there may be active destabilization/degradation from the cell surface when ligand is removed.

### Example 9. CD40L Regulation in T Cells

T cells were transduced with OT-001662 and then treated with 10 μM TMP on day 4. CD40L expression was analyzed by FACS both in the CD4 and CD8 positive T cell populations. The results are shown in Table 25.

TABLE 25
CD40L MFI in vitro

| Construct | CD4+ | CD8+ |
|---|---|---|
| Empty Vector | 781 | 190 |
| OT-001661 | 24489 | 14352 |
| OT-001662 (Vehicle) | 348 | 182 |
| OT-001662 (TMP) | 10159 | 4893 |

Ligand dependent regulation was observed with TMP treatment with CD40L levels less than what was observed with the constitutive CD40L construct namely OT-001661.

Based on the in vitro data, regulatability of CD40L constructs in vivo was tested. 10-week old NSG female mice were injected with T cells transduced with one of the following constructs empty vector, OT-001661, and OT-001662 (day 0). On day 1, mice were pre-bled prior to dose with ligand. On day 2, mice were dosed every 4 hours with TMP at 500 mg/kg body weight of the mouse or vehicle control. Two hours after every dose, blood was collected from the mice to measure CD40L levels. On day 3, i.e. 24 hours after the first dose, mice were terminally bled for analysis. The CD40L levels are shown in Table 26. The values in bold represent the average values for each bleed.

TABLE 26
% CD40L positive cells in vivo

| Construct | | | | | |
|---|---|---|---|---|---|
| (below)/Hours | | | | | |
| (Across) | 0 | 2 | 6 | 10 | 24 |
| EV | 2.2 | 5.1 | | | 5.2 |
| | 3.0 | 2.3 | | | 1.5 |
| | 3.9 | 3.2 | | | 0.7 |
| | 3.7 | 0.0 | | | 1.6 |
| | 3.2 | 2.6 | | | 2.2 |
| OT-001661 | 80.9 | 85.6 | | | 53.5 |
| | 87.1 | 67.4 | | | 66.7 |
| | 84.4 | 33.3 | | | 61.0 |
| | 107.8 | 62.7 | | | 41.9 |
| | 90.1 | 62.3 | | | 55.8 |
| OT-001662 | 0.8 | 0.6 | 0.3 | 0.0 | 0.0 |
| Vehicle | 0.9 | 1.0 | 0.4 | 0.0 | 0.3 |
| | 0.1 | 0.0 | 0.5 | 0.0 | 1.4 |
| | 1.8 | 1.9 | 0.2 | 0.3 | 0.7 |
| | 0.9 | 0.9 | 0.3 | 0.1 | 0.6 |
| OT-001662 | 0.9 | 11.8 | 22.1 | 26.0 | 1.2 |
| TMP | 0.4 | 13.0 | 25.7 | 21.4 | 0.8 |
| | 0.9 | 9.6 | 13.8 | 0.0 | 18.9 |
| | 1.8 | 0.0 | 18.0 | 33.1 | 27.2 |
| | 1.0 | 8.6 | 19.9 | 20.1 | 12.0 |

Peak CD40L expression of 20% was observed at 10 hours post initial dose. It was also noted in all instances that regulated CD40L expression was lower than the constitutively expressed construct after ligand exposure.

### Example 10. Dendritic Cell Activation

To test if CD40L is functionally able to activate dendritic cells in vivo, sequential intraperitoneal (IP) or intravenous (IV) injections of allogeneic moDCs and CD40L+/−T-cells into NSG mice were performed. The various groups utilized in the study are provided in Table 27. Construct OT-001661 was utilized for these experiments and results are shown in Table 28.

TABLE 27
Study Groups

| | T | | | |
|---|---|---|---|---|
| | Cell | DC | | |
| | # | # | Injection | |
| Gr. | ×106 | ×106 | Material | Timing of injection |
| 1 | — | — | Naïve | — |
| 2 | 6.9 | — | CD40L alone | Same day (Day 0) |
| 3 | 6.9 | 1 | CD40L (IV) + | Same day (Day 0) |
| | | | DC (IV) | |
| 4 | 6.9 | 5 | CD40L (IV) + | Same day (Day 0) |
| | | | DC (IV) | |
| 5 | 11.9 | 1 | CD40L (IV) + | 5 × 106 T Cells (Day |
| | | | DC (IP) | 0); 5 × 106 T Cells |
| | | | | (Day 1) |
| 6 | 6.9 | 1 | CD40L (IP) + | Same day (Day 0) |
| | | | DC (IP) | |
| 7 | 6.9 | 1 | CD40L (IV) + | Same day (Day 0) |
| | | | GMCSF/IL4 | |
| | | | pretreated DC | |
| | | | (IV) | |
| 8 | 6.9 | 1 | CD40L (IP) + | Same day (Day 0) |
| | | | GMCSF/IL4 | |
| | | | pretreated DC | |
| | | | (IP) | |

TABLE 28
Plasma IL12 levels (pg/ml)

| Days | 16 | 24 | 40 |
|---|---|---|---|
| Naive | — | 0.04 | — |
| T cell EV (IP) + DC | 0.73 | — | — |
| (IP) | 1.97 | — | — |
| | 2.67 | — | — |
| | 1.13 | — | — |
| T cell CD40L (IP) + | 237.07 | 152.53 | 59.04 |
| DC (IP) | 45.57 | 30.23 | 8.53 |
| | 121.73 | 68.25 | 27.33 |
| | 308.61 | 336.05 | 142.68 |
| T cell CD40L (IP) + | 3.26 | 3.75 | 1.31 |
| DC-stim (IP) | | | |

Results in Table 28 show that CD40L expressing T-cells can stimulate moDCs in-vivo to secrete detectable levels of IL12 suggesting that CD40L expressing T cells are able to activate dendritic cells leading to IL12 secretion.

### Example 11. Regulation of CD40L in T Cells by Different Drug Responsive Domains

To test regulation, activated T cells were lentivirally transduced with CD40L regulated by different drug responsive domains, activated T cells were lentivirally transduced with ecDHFR regulated CD40L (OT-001662), ER regulated CD40L (OT-001966), PDE5 regulated CD40L (OT-001892) and a control of CD40L (OT-001661). Two days later, cells were treated with vehicle or 100 mM ligand for 24h as described in Table 29 after which they were analyzed for CD40L surface expression. The results for CD4+ and CD8+ cells and total cells are shown below. In the table, “TMP” is trimethoprim, “Baz” is Bazedoxifene, “Vard” is Vardenafil and “DMSO” is dimethyl sulfoxide.

TABLE 29
Percent of cells expressing CD40L

| | | CD4+ | | CD8+ | | All Cells | |
|---|---|---|---|---|---|---|---|
| | | CD40L | | CD40L | | CD40L | |
| Construct (DD) | Ligand | positive | MFI | positive | MFI | positive | MFI |
| OT-001661 | N/A | 98.7% | 24489 | 97.8% | 14352 | 98% | 20451 |
| (CD40L | | | | | | | |
| control) | | | | | | | |
| OT-001662 | DMSO | 8.8% | 152 | 91.8% | 2.1 | 7.6% | 140 |
| (ecDHFR) | | | | | | | |
| OT-001662 | 10 uM | 94.4% | 2608 | 74.9% | 1070 | 89.7% | 2122 |
| (ecDHFR) | TMP | | | | | | |
| OT-001962 | DMSO | 58.3% | 548 | 4.1% | 157 | 46.5% | 434 |
| (hDHFR) | | | | | | | |
| OT-001962 | 100 uM | 83.5% | 1468 | 32.1% | 455 | 71.6% | 1109 |
| (hDHFR) | TMP | | | | | | |
| OT-001966 | DMSO | 17% | 197 | 2.2% | 95.6 | 14% | 174 |
| (ER) | | | | | | | |
| OT-001966 | 1 uM | 62.8% | 543 | 42.3% | 339 | 58.1% | 490 |
| (ER) | Baz | | | | | | |
| OT-001892 | DMSO | 11.% | 172 | 3.1% | 121 | 9.7% | 160 |
| (PDE5) | | | | | | | |
| OT-001892 | 100 uM | 53.7% | 494 | 75.2% | 745 | 70.6% | 682 |
| (PDE5) | Vard | | | | | | |

Regulated expression with all drug responsive domains significantly enhanced CD40L expression beyond endogenous levels. ecDHFR drug responsive domains show levels close to constitutive expression with ligand doses that are near clinically relevant levels.

### Example 12. CD40L Multimerization Mutants

Mutations were engineered within CD40L payload to reduce the binding affinity of CD40L payload for the CD40L endogenously expressed by cells. Constructs OT-002078, OT-002079, OT-002080, OT-002081, OT-002082 were generated with the CD40L trimerization mutants. HEK293T cells were transiently transfected with the constructs and CD40L expression was measured by FACS. Percentage of HEK293T cells that were positive for cell surface expression of CD40L are provided in Table 30.

TABLE 30
Percent of cells expressing CD40L

| Construct | % CD40L positive |
|---|---|
| OT-002078 | 49.7 |
| OT-002079 | 64.7 |
| OT-002080 | 0.11 |
| OT-002081 | 8.84 |
| OT-002082 | 0.028 |
| OT-001661 | 43.3 |
| Untransfected | 0.073 |
| Unstained | 0.070 |

Constructs OT-002078 and OT-002079 showed greater than 50% expression on cell surface. Since surface expression of CD40L relies on trimerization, these data suggest that the trimerization mutant proteins are able to still interact with each other resulting in their surface expression.

### Example 13. ER Regulated CD40L Expression

T cells were transduced with OT-001662 or OT-001666. On day 4, cells were treated with increasing doses of TMP for T cells expressing OT-001662. Cells expressing OT-001666 were treated either with increasing concentrations of Bazedoxifene or Raloxifene. The data are shown in Table 30 as the percentage of CD40L positive T cells. As shown in Table 31 and Table 32, both TMP and Bazedoxifene induced dose dependent increase in % CD40L positive T cells. Raloxifene dependent expression of OT-001666 was observed only at the 1 μM dose.

TABLE 31
Percent of cells expressing CD40L

| TMP Dose (μM) | OT-001662 |
|---|---|
| 100 | 73.3 |
| 10 | 71 |
| 1 | 38.8 |
| 0.1 | 13.3 |
| 0.01 | 8.74 |

TABLE 32
Percent of cells expressing CD40L

| Dose (μM) | Bazidoxefene | Raloxifene |
|---|---|---|
| 1 | 13.5 | 7.1 |
| 0.1 | 7.67 | 5.18 |
| 0.01 | 5.42 | 5 |
| 0.001 | 5.17 | 5.01 |
| 0.0001 | 4.88 | 4.67 |

To explore the effect of linker length in the DD mediated regulation of CD40L, constructs with varying linker lengths were generated. OT-001966, OT-001965, OT-001967, OT-001666 constructs were generated and are presented here in decreasing linker length order. HEK293T cells were transiently transfected with these constructs and treated with 1 μM Bazedoxifene for 24 hours. Median fluorescence intensity, an indicator of surface expression of CD40L, was analyzed by flow cytometry. All constructs tested showed Bazedoxifene dependent regulation of CD40L expression indicating that all linkers tested allow for regulation. The longest linker-based construct OT-001966, resulted in the highest percentage of cells with high CD40L surface expression.

Jurkat cells were lentivirally transduced and 24 hours after transduction were treated with DMSO or 1 μM Bazedoxifene for another 24 hours and analyzed by flow cytometry for surface expression of CD40L. Table 33 shows the percentage of CD40L positive cells. In Table 33, SR indicates stabilization ratio.

TABLE 33
Percent of Jurkat cells expressing CD40L

| | | 1 μM | |
|---|---|---|---|
| Construct | DMSO | Bazedoxifene | SR |
| OT-001966 | 3 | 46 | 15.3 |
| OT-001965 | 3 | 27 | 9 |
| OT-001967 | 2 | 30 | 15 |
| OT-001666 | 10 | 30 | 3 |

As shown in Table 33, the longest linker-based construct OT-001966, resulted in the highest percentage of cells with high CD40L surface expression. This trend was also observed in the stabilization ratio calculations.

T cells were transduced with OT-001966, OT-001967, OT-001666. On day 4, cells were analyzed by flow cytometry. Similar to the experiments performed in HEK293T and Jurkat cells, longer linker enhanced surface expression with ER regulated CD40L constructs.

Dose response with Bazedoxifene in T cells were conducted for OT-001966, OT-001967, OT-001666. Expression of CD40L was measured at (a) day 4 after transduction, (b) after freezing and thawing the cells or (c) after freezing and thawing the cells followed by restimulation with CD3/CD28 beads. The CD40L MFI (median fluorescence intensity) within CD4 and CD8 positive subpopulations is shown in Table 34 and Table 35 respectively.

TABLE 34
CD40L expression in CD4+ cells

| | OT-001666 | | | OT-001967 | | | OT-001966 | | |
|---|---|---|---|---|---|---|---|---|---|
| Dose | Post | | Day | Post | | Day | Post | | Day |
| (nM) | Thaw | Restim | 4 | Thaw | Restim | 5 | Thaw | Restim | 4 |
| 0.01 | 134 | 592 | 372 | 156 | 913 | 473 | 183 | 340 | 415 |
| 0.1 | 138 | 658 | 420 | 160 | 922 | 474 | 185 | 340 | 499 |
| 1 | 145 | 682 | 427 | 161 | 913 | 483 | 193 | 364 | 545 |
| 10 | 162 | 658 | 476 | 172 | 1131 | 497 | 213 | 472 | 616 |
| 100 | 208 | 953 | 732 | 200 | 1549 | 630 | 258 | 561 | 1028 |
| 1000 | 241 | 1688 | 1397 | 223 | 1232 | 998 | 292 | 741 | 1551 |
| 10000 | 537 | 1577 | 1274 | 457 | 291 | 1000 | 577 | 447 | 1095 |

TABLE 35
CD40L expression in CD8+ cells

| | OT-001666 | | | OT-001967 | | | OT-001966 | | |
|---|---|---|---|---|---|---|---|---|---|
| Dose | Post | | Day | Post | | Day | Post | | Day |
| (nM) | Thaw | Restim | 4 | Thaw | Restim | 5 | Thaw | Restim | 4 |
| 0.01 | 103 | 238 | 165 | 108 | | 153 | 113 | 340 | 168 |
| 0.1 | 105 | 244 | 171 | 105 | 269 | 153 | 117 | 340 | 176 |
| 1 | 107 | 263 | 172 | 108 | 274 | 157 | 121 | 364 | 190 |
| 10 | 117 | 253 | 176 | 114 | 265 | 158 | 148 | 472 | 212 |
| 100 | 170 | 354 | 313 | 149 | 326 | 226 | 204 | 561 | 442 |
| 1000 | 213 | 567 | 601 | 178 | 445 | 424 | 230 | 741 | 669 |
| 10000 | 326 | 458 | 404 | 263 | 372 | 392 | 375 | 447 | 438 |

As shown in Table 34 and Table 35, Bazedoxifene affected cell health at highest dose. Restimulation of T cells was necessary for higher expression after freeze thaw. Within the CD8 positive cells, highest ER regulated CD40L expression was observed with long linker OT-001966 and restimulation.

### Example 14. Human and E. coli DHFR Regulated CD40L Expression

HEK 293T cells were transiently transduced with hDHFR regulated constructs namely OT-001962, OT-001961, OT-001963 for 24 hours. Following transfection, cells were treated with 50 μM TMP for 24 hours and CD40L cell surface expression was measured using flow cytometry. Table 36 shows % CD40L positive cells with TMP treatment. CD40L expression in the absence of ligand was virtually undetectable.

TABLE 36
% CD40L expression with TMP treatment

| | % |
|---|---|
| Construct | CD40L |
| OT-001962 | 14.3 |
| OT-001961 | 14.6 |
| OT-001963 | 13.3 |

The data in Table 36 show that CD40L can be regulated by hDHFR DDs and TMP.

Jurkat cells were transduced with lentiviruses corresponding to OT-001662, OT-001962, OT-001961, OT-001963. 24 hours after transduction, cells were split and treated with DMSO or 50 μM TMP for another 24 hours until flow cytometry-based analysis. The results are shown in Table 37 where SR indicates stabilization ration.

TABLE 37
% CD40L expression with TMP treatment

| Construct | Vehicle | TMP | SR |
|---|---|---|---|
| OT-001662 | 1 | 90 | 90 |
| OT-001962 | 3 | 50 | 16.7 |
| OT-001961 | 2 | 3.5 | 1.8 |
| OT-001963 | 2 | 20 | 10 |

Among the hDHFR constructs tested, OT-001962 showed strong TMP dependent regulation and the highest stabilization ratio.

Dose response with TMP in T cells were conducted for OT-001962. Expression of CD40L was measured at (a) day 4 after transduction, (b) after freezing and thawing the cells or (c) after freezing and thawing the cells followed by restimulation with CD3/CD28 beads. The CD40L MFI (median fluorescence intensity) within CD4 and CD8 positive subpopulations is shown in Table 38.

TABLE 38
CD40L expression with OT-001962

| | CD4+ | | | CD8+ | | |
|---|---|---|---|---|---|---|
| Dose | Post | | Day | Post | | Day |
| (nM) | Thaw | Restim | 4 | Thaw | Restim | 4 |
| 0.1 | 153 | 631 | 388 | 111 | 254 | 181 |
| 1 | 156 | 647 | 396 | 116 | 257 | 178 |
| 10 | 158 | 645 | 405 | 114 | 258 | 182 |
| 100 | 165 | 644 | 388 | 117 | 255 | 182 |
| 1000 | 166 | 737 | 480 | 118 | 258 | 204 |
| 10000 | 186 | 1382 | 1067 | 129 | 382 | 393 |
| 100000 | 395 | 6275 | 5917 | 236 | 1185 | 1941 |

As shown in Table 38, restimulation after freeze and thaw was required to achieve CD40L expression that was comparable to the expression obtained 4 days after transduction.

HEK293T cells were transiently transfected with the constructs in Table 39 (1 μg of DNA). 24 hours after transfection, the media was removed and replaced with fresh medium containing 50 μM TMP or DMSO. Another 24 hours later cells were analyzed by flow cytometry for CD40L surface expression. Table 39 provides CD40L expression as both % CD40L positive cells and the Median fluorescence intensity (MFI) values.

TABLE 39
CD40L expression with CD40L-ecDHFR constructs

| | | | | SR (for | SR |
|---|---|---|---|---|---|
| | | % | MFI | % | (for |
| Construct | Ligand | CD40L | (Median) | CD40L) | MET) |
| OT-002021 | TMP | 70.7 | 18589 | 2.98 | 3.31 |
| | Vehicle | 23.7 | 5616 | | |
| OT-002022 | TMP | 70.5 | 19941 | 2.96 | 2.89 |
| | Vehicle | 23.8 | 6896 | | |
| OT-002023 | TMP | 62.4 | 16902 | 3.55 | 2.84 |
| | Vehicle | 17.6 | 5956 | | |
| OT-002024 | TMP | 53.9 | 12938 | 3.05 | 1.93 |
| | Vehicle | 17.7 | 6718 | | |
| OT-002025 | TMP | 61.7 | 16230 | 1.41 | 2.24 |
| | Vehicle | 43.7 | 7241 | | |
| OT-002026 | TMP | 63.4 | 17721 | 3.66 | 2.45 |
| | Vehicle | 17.3 | 7241 | | |
| OT-002027 | TMP | 70.7 | 19679 | 2.84 | 2.58 |
| | Vehicle | 24.9 | 7619 | | |
| OT-002028 | TMP | 69.1 | 16196 | 2.81 | 2.42 |
| | Vehicle | 24.6 | 6705 | | |
| OT-002029 | TMP | 45 | 7720 | 2.07 | 1.03 |
| | Vehicle | 21.7 | 7519 | | |
| OT-002030 | TMP | 64.9 | 14855 | 1.95 | 1.92 |
| | Vehicle | 33.3 | 7734 | | |
| OT-002031 | TMP | 64.8 | 15856 | 2.44 | 2.16 |
| | Vehicle | 26.6 | 7351 | | |
| OT-002032 | TMP | 68.7 | 21363 | 3.37 | 2.88 |
| | Vehicle | 20.4 | 7407 | | |
| OT-002033 | TMP | 17.3 | 5521 | 1.63 | 0.93 |
| | Vehicle | 10.6 | 5933 | | |
| OT-002034 | TMP | 71.1 | 20569 | 2.44 | 2.59 |
| | Vehicle | 29.1 | 7957 | | |
| OT-002035 | TMP | 23.8 | 8311 | 0.89 | 0.97 |
| | Vehicle | 26.7 | 8535 | | |
| OT-002036 | TMP | 69.5 | 17914 | 2.37 | 2.66 |
| | Vehicle | 29.3 | 6730 | | |
| OT-002037 | TMP | 63.5 | 13928 | 2.08 | 2.00 |
| | Vehicle | 30.6 | 6961 | | |
| OT-002038 | TMP | 67.9 | 20029 | 1.86 | 2.89 |
| | Vehicle | 36.6 | 6922 | | |
| OT-002039 | TMP | 64.2 | 16404 | 1.83 | 2.10 |
| | Vehicle | 35.1 | 7793 | | |
| OT-002040 | TMP | 65.8 | 16938 | 1.65 | 2.18 |
| | Vehicle | 40 | 7778 | | |

With the exception of OT-002035, all constructs tested, showed TMP mediated expression of CD40L.

### Example 15. Regulation of Cleavage Resistant CD40L

T cells were transduced with OT-001668 and on day 4, the percentage of CD40L expressing T cells (within the both CD4 and CD8) subpopulations was calculated. The results are shown in Table 40.

TABLE 40
CD40L expression

| Construct | CD4+ | CD8+ |
|---|---|---|
| Empty Vector | 20.6 | 0.51 |
| OT-001668 | 95.5 | 86.7 |

High expression of CD40L was observed with the cleavage resistant construct OT-001668. Some expression was observed with the T cells transduced with empty vector, which is likely the expression of endogenous CD40L expressed by the CD4+ T cells. T cells were transduced with ecDHFR regulated construct OT-001671 that include a CD40L protein that was resistant to cleavage. On day 4 cells were treated with 10 μM TMP for 24 hours. TMP-mediated Regulation of CD40L expression was observed in both the CD4 and the CD8 T cell populations. In an independent experiment, T cells transduced with varying volumes of virus corresponding to OT-001671 were treated with increasing doses of TMP for 24 hours. The percentage of CD40L positive cells is shown in Table 41. In Table 41, 0.5 μL, 2 μL, and 10 μL indicate the different volumes of virus that were used.

TABLE 41
TMP dose response

| TMP dose | CD4+ | | | CD8+ | | |
|---|---|---|---|---|---|---|
| (μM) | 0.5 μL | 2 μL | 10 μL | 0.5 μL | 2 μL | 10 μL |
| 0.001 | 17.9 | 11.8 | 4.74 | 0.59 | 0.6 | 1.18 |
| 0.01 | 18.8 | 12.2 | 6.79 | 0.56 | 0.59 | 0.89 |
| 0.1 | 20.4 | 14.9 | 7.65 | 0.45 | 0.88 | 0.95 |
| 1 | 20.3 | 17.6 | 17.2 | 0.43 | 1.29 | 2.87 |
| 10 | 27.5 | 30 | 43.7 | 2.44 | 5.57 | 12.4 |

ecDHFR regulated cleavage resistant CD40L demonstrated an increase in the percentage of CD40L positive cells in response to TMP dose increments. Higher levels of virus added (0.5, 2, or 10 μl) increased CD40L levels at higher doses of TMP. Similar experimental trends were observed with the measurements of median fluorescent intensity values.

### Example 16. In Vivo CD40L Regulation in Jurkat Cells

Jurkat cells stably expressing OT-001661, OT-002034 were intravenously infused into 8-week old NSG mice (20 million cells per mouse). Starting by day 15, mice were orally dosed according to the study design shown in Table 42 and Jurkat cells were harvested on day 16 from the bone marrow and analyzed by FACS. In Tables 41 and 42, TMP refers to Trimethoprim and all dosing is relative to “0 hr” time point on day 16. The results are shown in Table 43 and Table 44 where “EV” refers to empty vector control.

TABLE 42
Study design

| | | Jurkat | | |
|---|---|---|---|---|
| | Mice | Cells | | Ligand (dosing relative to “0 hr” |
| Gr. | (n) | (×106) | Vector (Name) | time point on day 16 |
| 1 | 4 | 20 | Parental Jurkat | Vehicle (1 dose at −20 h, 1x dose at |
| | | | cells | 0 h) |
| 2 | 4 | 20 | OT-001661 | Vehicle (1x dose at −20 h, 1x dose |
| | | | | at 0 h) |
| 3 | 4 | 20 | OT-002034 | Vehicle (3x dose on day 16 at 0, 4, |
| | | | (ecDHFR) | 8 h) |
| 4 | 4 | 20 | OT-002034 | TMP 500 mg/kg (3x dose on day |
| | | | (ecDHFR) | 16 at 0, 4, 8 h) |

TABLE 43
CD40L MFI values

| | | | OT-002034 | OT-002034 |
|---|---|---|---|---|
| Description | EV | OT-001661 | Vehicle | TMP |
| Mouse 1 | 145 | 507 | 101 | 608 |
| Mouse 2 | 153 | 572 | 80.6 | 542 |
| Mouse 3 | 121 | 660 | 97.9 | 505 |
| Mouse 4 | 158 | 607 | 95.1 | 428 |
| Average | 144.25 | 586.5 | 93.65 | 520.75 |

TABLE 44
Percent CD40L positive cells

| | | | OT-002034 | OT-002034 |
|---|---|---|---|---|
| Description | EV | OT-001661 | Vehicle | TMP |
| Mouse 1 | 9.27 | 52.5 | 2.36 | 61 |
| Mouse 2 | 9.11 | 57.5 | 0.83 | 56.3 |
| Mouse 3 | 5.46 | 64.2 | 2.17 | 52.2 |
| Mouse 4 | 10.4 | 61.4 | 1.92 | 44.1 |
| Average | 8.56 | 58.9 | 1.82 | 53.4 |

As shown in Table 43 and Table 44, OT-002034 expressing Jurkat cells retrieved from bone marrows of mice treated with TMP showed increased CD40L values and % CD40L positive cells when compared to mice treated with vehicle control, or empty vector, but comparable to Jurkat cells expressing OT-001661.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising an effector module, said effector module comprising a stimulus response element (SRE) operably linked to a payload, wherein said payload comprises a mutant CD40L comprising a deletion of S110-G116 relative to SEQ ID NO:3820 or one or more mutations selected from Y170G, Y172G, H224G, G226F, G226H, G226W, or G227F relative to SEQ ID NO: 3820.

2. The composition of claim 1, wherein said SRE comprises, in whole or in part, a DRD selected from an ER, an ecDHFR, a FKBP, a PDE5, or a hDHFR protein, wherein the DRD comprises one or more mutations.

3. The composition of claim 2, wherein the DRD is derived from an ER protein and wherein the DRD has an amino acid sequence comprising SEQ ID NO: 633, 637, 641, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, or 726.

4. The composition of claim 2, wherein the DRD is derived from an ecDHFR protein, and wherein the DRD has an amino acid sequence comprising SEQ ID NO: 253, 255, 264, 267, 269, 6554, 6556, 6558, 6560, 6562, 6564, 6566, 6568, 6570, 6572, 6574, 6576, 6578, 6580, 6582, 6584, 6586, 6588, or 6590.

5. The composition of claim 2, wherein the DRD is derived from a FKBP protein, and wherein the DRD has an amino acid sequence comprising SEQ ID NO: 270, 271, 272, 274, 277, 285 or 286.

6. The composition of claim 2, wherein the DRD is derived from a PDE5 protein, and wherein the DRD has an amino acid sequence comprising SEQ ID NO: 294, 296, 298, 300, 302, 306, 308, 313, 315, 317, 318, 319, 320, 321, 322, 323, 324, 325, 326, 327, 328, 329, 330, 331, 332, 333, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 375, 378, 381, 384, 387, 389, 391, 394, 397, 400, 403, 406, 409, 412, 415, 417, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 6406, 6408, 6410, 6412, 6414, 6416, 6418, 6420, 6422, 6424, 6426, 6428, 6430, 6432, 6434, 6436, 6438, 6440, 6442, 6444, 6446, 6448, 6450, 6452, 6454, 6456, 6458, 6460, 6462, 6464, 6466, 6468, 6470, 6472, 6474, 6476, 6478, 6480, 6482, 6484, 6486, 6488, 6490, 6492, 6494, 6496, 6498, 6500, 6502, 6504, 6506, 6508, 6510, 6512, 6514, 6516, 6518, 6520, 6522, 6524, 6526, 6528, or 6530.

7. The composition of claim 2, wherein the DRD is derived from an hDHFR protein, and wherein the DRD has an amino acid sequence comprising SEQ ID NO: 78, 80, 82, 84, 86, 88, 90, 92, 94, 96, 98, 100, 102, 104, 106, 108, 110, 112, 114, 116, 118, 119, 121, 123, 125, 127, 129, 131, 133, 135, 137, 139, 141, 143, 145, 149, 151, 153, 155, 157, 159, 161, 163, 165, 166, 168, 170, 172, 174, 176, 179, 181, 183, 187, 189, 191, 193, 195, 197, 201, 203, 205, 207, 209, 210, 212, 214, 216, 218, 221, 223, 225, 227, 229, 231, 233, 235, 237, 239, 241, 243, 245, 247, 249, 251, or 6552.

8. The composition of claim 1, wherein the DRD is responsive to or interacts with at least one stimulus.

9. The composition of claim 8, wherein the stimulus is a small molecule.

10-12. (canceled)

13. An effector module, said effector module comprising a drug responsive element (DRD) operably linked to a payload, wherein said DRD comprises, in whole or in part, an ER, an ecDHFR, a FKBP, a PDE5, or an hDHFR protein comprising one or more mutations and wherein said payload comprises in whole or in part a human CD40L comprising one or more mutations.

14. The effector module of claim 13, wherein the effector module comprises an amino acid sequence selected from: SEQ ID NO: 6546, or SEQ ID NO: 6550, or SEQ ID NO: 6620, or SEQ ID NO: 6622, or SEQ ID NO: 6624, or SEQ ID NO: 6626, or SEQ ID NO: 6628, or SEQ ID NO: 6630, or SEQ ID NO: 6632, or SEQ ID NO: 6634, or SEQ ID NO: 6636, or SEQ ID NO: 6638, or SEQ ID NO: 6640, or SEQ ID NO: 6642, or SEQ ID NO: 6644, or SEQ ID NO: 6646, or SEQ ID NO: 6648, or SEQ ID NO: 6650, or SEQ ID NO: 6652, or SEQ ID NO: 6654, or SEQ ID NO: 6656, or SEQ ID NO: 6658, or SEQ ID NO: 6660, or SEQ ID NO: 6662, or SEQ ID NO: 6664, or SEQ ID NO: 6666, or SEQ ID NO: 6668, or SEQ ID NO: 6670, or SEQ ID NO: 6672.

15. (canceled)

16. The effector module of claim 13, wherein the DRD comprises an amino acid sequence of SEQ ID NO: 253, 255, 264, 267, 269, 6554, 6556, 6558, 6560, 6562, 6564, 6566, 6568, 6570, 6572, 6574, 6576, 6578, 6580, 6582, 6584, 6586, 6588, or 6590.

17. The effector module of claim 13, wherein the DRD comprises an amino acid sequence of SEQ ID NO: 633, 637, 641, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, or 726.

18. The effector module of claim 13, wherein the DRD comprises an amino acid sequence of SEQ ID NO: 270, 271, 272, 274, 277, 285 or 286.

19. The effector module of claim 13, wherein the DRD comprises an amino acid sequence of SEQ ID NO: 294, 296, 298, 300, 302, 306, 308, 313, 315, 317, 318, 319, 320, 321, 322, 323, 324, 325, 326, 327, 328, 329, 330, 331, 332, 333, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 375, 378, 381, 384, 387, 389, 391, 394, 397, 400, 403, 406, 409, 412, 415, 417, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 6406, 6408, 6410, 6412, 6414, 6416, 6418, 6420, 6422, 6424, 6426, 6428, 6430, 6432, 6434, 6436, 6438, 6440, 6442, 6444, 6446, 6448, 6450, 6452, 6454, 6456, 6458, 6460, 6462, 6464, 6466, 6468, 6470, 6472, 6474, 6476, 6478, 6480, 6482, 6484, 6486, 6488, 6490, 6492, 6494, 6496, 6498, 6500, 6502, 6504, 6506, 6508, 6510, 6512, 6514, 6516, 6518, 6520, 6522, 6524, 6526, 6528, or 6530.

20. The effector module of claim 13, wherein the DRD comprises an amino acid sequence of SEQ ID NO: 78, 80, 82, 84, 86, 88, 90, 92, 94, 96, 98, 100, 102, 104, 106, 108, 110, 112, 114, 116, 118, 119, 121, 123, 125, 127, 129, 131, 133, 135, 137, 139, 141, 143, 145, 149, 151, 153, 155, 157, 159, 161, 163, 165, 166, 168, 170, 172, 174, 176, 179, 181, 183, 187, 189, 191, 193, 195, 197, 201, 203, 205, 207, 209, 210, 212, 214, 216, 218, 221, 223, 225, 227, 229, 231, 233, 235, 237, 239, 241, 243, 245, 247, 249, 251, or 6552.

21. The effector module of claim 13, wherein the DRD is responsive to or interacts with at least one stimulus.

22. The effector module of claim 20, wherein the stimulus is a small molecule, and said molecule is TMP or MTX.

23. The effector module of claim 19, wherein the stimulus is a small molecule, and said small molecule is Vardenafil, Tadalafil or Sildenafil.

24. The effector module of claim 17, wherein the stimulus is a small molecule, said small molecule is Bazedoxifene, Raloxifene, or Shield-1.

25.-44. (canceled)
